SEQUENCE LISTING (3 sequences)

SEQ ID NO: 1 - protein, 736 aa, Adeno-associated virus 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 2 - protein, 599 aa, Adeno-associated virus 9
TAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 3 - protein, 534 aa, Adeno-associated virus 9
MASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

(19) United States
(12) Patent Application Publication
(10) Pub. No.: US 2022/0267796 A1
(43) Pub. Date: Aug. 25, 2022

(54) ADENO-ASSOCIATED VIRUS PURIFICATION METHODS

(71) Applicant: TAKEDA PHARMACEUTICAL COMPANY LIMITED, OSAKA (JP)

(72) Inventors: Christian FIEDLER, Vienna (AT); Marcus SCHEINDEL, Vienna (AT); Meinhard HASSLACHER, Vienna (AT); Jadranka KOEHN, Laupheim (DE)

(73) Assignee: TAKEDA PHARMACEUTICAL COMPANY LIMITED, OSAKA (JP)

(21) Appl. No.: 17/622,044

(22) PCT Filed: Jun. 26, 2020
(86) PCT No.: PCT/US2020/039971

§ 371 Date: Dec. 22, 2021

Related U.S. Application Data
(60) Provisional application No. 62/868,282, filed on Jun. 28, 2019.

(51) Int. Cl. C12N 15/86; B01D 15/16; B01D 15/38

(52) U.S. Cl. CPC C12N 15/86; B01D 15/161; B01D 15/3804; C12N 2750/14122; C12N 2750/14143; C12N 2750/14151

(57) ABSTRACT

Provided herein are methods of producing an adeno-associated virus (AAV) product and methods of purifying adeno-associated virus. AAV is loaded onto an affinity resin, wash steps are undertaken at room temperature, and AAV is eluted from the affinity resin at a lower temperature. Various buffers are disclosed for use in the wash steps and elution.

### CROSS-REFERENCE TO RELATED APPLICATIONS

This application claims priority to U.S. Provisional Application No. 62/868,282, filed Jun. 28, 2019, the disclosure of which is herein incorporated by reference in its entirety.

### TECHNICAL FIELD

The invention relates to materials and methods of purifying adeno-associated virus (AAV).

### BACKGROUND

Adeno-associated virus (AAV) is a small, non-enveloped virus that packages a linear single-stranded DNA genome. AAV belongs to the family Parvoviridae and the genus Dependovirus, since productive infection by AAV occurs only in the presence of a helper virus, such as, for example, adenovirus or herpes virus.

For AAV to be safely used in the clinic, AAV has been genetically modified at several locations within its genome. For example, the Rep gene, which is required for viral replication, and the element required for site-specific integration have been eliminated from the AAV genome in many viral vectors. Such recombinant AAV (rAAV) exist in an extrachromosomal state and have very low integration efficiency into the genomic DNA. The possibility of rAAV inducing random mutagenesis in a host cell is thus reduced, if not eliminated altogether. Because of these properties and the lack of pathogenicity, rAAV has shown great promise as a gene therapy vector in multiple aspects of pre-clinical and clinical applications.

Though the effort to design efficient, large-scale methods to purify an AAV product suitable for human administration has been great, there remains a need for better AAV purification methods. There are various other proteins and materials from the host cell culture matrix that could be more efficiently removed during the purification of AAV. AAV purification methods which include steps for removing host cell material from the final AAV product are therefore desired.

### SUMMARY

A feature of AAV vector generation in cell culture is the formation of a complex matrix that comprises material from disrupted cells. In particular, host cell proteins, proteasomes, cell debris and potential virus-specific receptors are often present in the material from disrupted cells. The disclosed methods which include steps for removing host cell material from the final AAV product in conditions that result in greater purity at a physiologically applicable pH.

In one aspect, provided herein is a method of purifying an adeno-associated virus (AAV) comprising

(a) loading an AAV containing solution onto an affinity resin targeted against an epitope on the AAV under conditions that allow binding between the AAV in the solution and the affinity resin;

(b) undertaking at least one wash step at room temperature; and

(c) eluting the AAV from the affinity resin at a temperature of less than 18° C.

Room temperature is between 18-26° C. Room temperature may be 18° C., 18.5° C., 19° C., 19.5° C., 20° C., 20.5° C., 21° C., 21.5° C., 22° C., 22.5° C., 23° C., 23.5° C., 24° C., 24.5° C., 25° C., 25.5° C. or 26° C. Room temperature may be 18° C., about 18.5° C., about 19° C., about 19.5° C., about 20° C., about 20.5° C., about 21° C., about 21.5° C., about 22° C., about 22.5° C., about 23° C., about 23.5° C., about 24° C., about 24.5° C., about 25° C., about 25.5° C. or 26° C.

In some embodiments, the temperature in step (c) is between 1° C. and 12° C. In some embodiments, the temperature in step (c) is between 2° C. and 8° C. In some embodiments, the temperature in step (c) is 1° C., 1.5° C., 2° C., 2.5° C., 3° C., 3.5° C., 4° C., 4.5° C., 5° C., 5.5° C., 6° C., 6.5° C., 7° C., 7.5° C., 8° C., 8.5° C., 9° C., 9.5° C., 10° C., 10.5° C., 11° C., 11.5° C., or 12° C. In some embodiments, the temperature in step (c) is about 1° C., about 1.5° C., about 2° C., about 2.5° C., about 3° C., about 3.5° C., about 4° C., about 4.5° C., about 5° C., about 5.5° C., about 6° C., about 6.5° C., about 7° C., about 7.5° C., about 8° C., about 8.5° C., about 9° C., about 9.5° C., about 10° C., about 10.5° C., about 11° C., about 11.5° C., or about 12° C.

In some embodiments, the method further comprises contacting the AAV containing solution with an anion exchanger and eluting the AAV containing solution from the anion exchanger prior to loading the AAV containing solution onto the affinity resin.

In some embodiments, the AAV obtained from the eluting step has an HC impurity level of s 99.9%. In some embodiments, the AAV obtained from the eluting step has an HC impurity level of s 99.0%.

In some embodiments, the AAV is AAV9. In some embodiments, the AAV9 comprises a wild-type VP1. In some embodiments, the AAV9 comprises a VP1 of SEQ ID NO: 1.

In some embodiments, the method further comprises contacting the AAV containing solution with a filter comprising positively charged groups effective to deplete acidic charged contaminants from the AAV containing solution.

In some embodiments, the method further comprises nanofiltration of an AAV fraction to remove viruses greater than 35 nm.

In some embodiments, the method further comprises a polish step comprising performing cation exchange chromatography.

In some embodiments, the method further comprises testing an AAV fraction via an AAV-specific ELISA.

In some embodiments, the AAV specific ELISA is a sandwich ELISA specific for AAV.

In another aspect, provided herein is an AAV product produced by any of the methods described above.

In one aspect, provided herein is a method of purifying an adeno-associated virus (AAV) comprising: (a) loading an AAV containing solution onto an affinity resin targeted against the AAV at room temperature and under conditions that allow binding between the AAV in the solution and the affinity resin; (b) undertaking at least one wash step at room temperature; and (c) eluting the AAV from the affinity resin at a temperature of less than 18° C.

In some embodiments, the temperature in step (c) is between 1° C. and 12° C. In some embodiments, the temperature in step (c) is between 2° C. and 8° C.

In some embodiments, the method further comprises contacting the AAV containing solution with an anion exchanger and eluting the AAV containing solution from the anion exchanger prior to loading the AAV containing solution onto the affinity resin.

In some embodiments, at least two wash steps are performed at room temperature. In some embodiments, at least three wash steps are performed at room temperature. In some embodiments, at least four wash steps are performed at room temperature.

In some embodiments, two wash steps are performed. In some embodiments, three wash steps are performed. In some embodiments, four wash steps are performed.

In some embodiments, the wash steps are performed in succession.

In some embodiments, at least one wash buffer comprises from about 10 to about 200 mM TrisHCl and from about 50 to about 500 mM salt. In some embodiments, at least one wash buffer comprises from about 25 to about 100 mM TrisHCl and from about 75 to about 250 mM salt. In some embodiments, at least one wash buffer comprises from about 40 to about 60 mM TrisHCl and from about 100 to about 150 mM salt. In some embodiments, at least one wash buffer comprises about 50 mM TrisHCl and about 125 mM salt. In some embodiments, the wash buffer has a pH from about 7.5 to about 9.2, about 8.0 to about 9.0, or about 8.0 to about 8.8, or about 8.5. In some embodiments, at least one wash buffer comprises about 50 mM TrisHCl and about 125 mM salt and has a pH of about 8.5.

In some embodiments, at least one wash buffer comprises from about 10 to about 2000 mM sodium acetate and from about 0.001 to about 1% (w/w) polysorbate 80. In some embodiments, at least one wash buffer comprises from about 50 to about 200 mM sodium acetate and from about 0.005 to about 0.3% (w/w) polysorbate 80. In some embodiments, at least one wash buffer comprises from about 90 to about 110 mM sodium acetate and from about 0.05 to about 0.2% (w/w) polysorbate 80. In some embodiments, at least one wash buffer comprises about 100 mM sodium acetate and about 0.1% (w/w) polysorbate 80. In some embodiments, the wash buffer has a pH from about 5.0 to about 7.4, about 5.5 to about 7.0, or about 5.5 to about 6.5, or about 6.0. In some embodiments, at least one wash buffer comprises about 100 mM sodium acetate and about 0.1% (w/w) polysorbate 80, and has a pH of about 6.0.

In some embodiments, at least one wash buffer comprises from about 10 to about 200 mM TrisHCl and from about 10 to about 75% (w/w) ethylene glycol. In some embodiments, at least one wash buffer comprises from about 25 mM to about 100 mM TrisHCl and from about 25% to about 70% (w/w) ethylene glycol. In some embodiments, at least one wash buffer comprises from about 40 mM to about 60 mM TrisHCl and from about 40% to about 60% (w/w) ethylene glycol. In some embodiments, at least one wash buffer comprises about 50 mM TrisHCl and about 50% (w/w) ethylene glycol. In some embodiments, the wash buffer has a pH from about 7.5 to about 9.2, about 8.0 to about 9.0, or about 8.0 to about 8.8, or about 8.5. In some embodiments, at least one wash buffer comprises about 50 mM TrisHCl and about 50% (w/w) ethylene glycol, and has a pH of about 8.5.

In some embodiments, at least one wash buffer comprises from about 10 to about 200 mM glycine, about 1 to about 100 mM histidine, about 20 to about 500 mM salt, about 1 to about 10% (w/w) trehalose and about 0.0005 to about 1% (w/w) polysorbate 80. In some embodiments, at least one wash buffer comprises from about 30 mM to about 80 mM glycine, about 5 to about 20 mM histidine, about 50 to about 200 mM salt, about 3 to about 8% (w/w) trehalose and about 0.001 to about 0.1% (w/w) polysorbate 80. In some embodiments, at least one wash buffer comprises from about 40 to about 60 mM glycine, about 5 to about 15 mM histidine, about 90 to about 110 mM salt, about 4 to about 6% (w/w) trehalose and about 0.001 to about 0.05% (w/w) polysorbate 80. In some embodiments, at least one wash buffer comprises about 50 mM glycine, about 10 mM histidine, about 100 mM salt, about 5% (w/w) trehalose, about 0.005% (w/w) polysorbate 80. In some embodiments, the wash buffer has a pH from about 6.0 to about 8.0, about 6.5 to about 7.5, or about 7.0 to about 7.4, or about 7.0. In some embodiments, at least one wash buffer comprises about 50 mM glycine, about 10 mM histidine, about 100 mM salt, about 5% (w/w) trehalose, about 0.005% (w/w) polysorbate 80, and has a pH of about 7.0.

In some embodiments, at least one wash buffer comprises from about 1 to about 200 mM TrisHCl, from about 50 to about 500 mM salt, and from about 0.001 to about 1% (w/w) polysorbate 80. In some embodiments, at least one wash buffer comprises from about 5 to about 50 mM TrisHCl, from about 75 to about 250 mM salt, and from about 0.005 to about 0.3% (w/w) polysorbate 80. In some embodiments, at least one wash buffer comprises from about 10 to about 30 mM TrisHCl, from about 140 to about 160 mM salt, and from about 0.05 to about 0.2% (w/w) polysorbate 80. In some embodiments, at least one wash buffer comprises about 20 mM TrisHCl, about 150 mM salt, and about 0.1% (w/w) polysorbate 80. In some embodiments, the wash buffer has a pH from about 6.0 to about 8.8, about 6.5 to about 8.5, or about 7.0 to about 8.0, or about 7.4. In some embodiments, at least one wash buffer comprises about 20 mM TrisHCl, about 150 mM salt, and about 0.1% (w/w) polysorbate 80, and has a pH of about 7.4.

In some embodiments, step (c) comprises eluting the AAV with at least one elution buffer. In some embodiments, at least one elution buffer is the same as at least one of the wash buffers. In some embodiments, at least one elution buffer is the same as the last wash buffer used in the final wash step before eluting the AAV in step (c). In some embodiments, the first elution buffer is the same as the last wash buffer used in the final wash step before eluting the AAV in step (c).

In some embodiments, at least one elution buffer comprises from about 10 to about 200 mM TrisHCl and from about 50 to about 500 mM salt. In some embodiments, at least one elution buffer comprises from about 25 to about 100 mM TrisHCl and from about 75 to about 250 mM salt. In some embodiments, at least one elution buffer comprises from about 40 to about 60 mM TrisHCl and from about 100 to about 150 mM salt. In some embodiments, at least one elution buffer comprises about 50 mM TrisHCl and about 125 mM salt. In some embodiments, the elution buffer has a pH from about 7.5 to about 9.2, about 8.0 to about 9.0, or about 8.0 to about 8.8, or about 8.5. In some embodiments, at least one elution buffer comprises about 50 mM TrisHCl and about 125 mM salt and has a pH of about 8.5.

In some embodiments, at least one elution buffer comprises from about 10 to about 2000 mM sodium acetate and from about 0.001 to about 1% (w/w) polysorbate 80. In some embodiments, at least one elution buffer comprises from about 50 to about 200 mM sodium acetate and from about 0.005 to about 0.3% (w/w) polysorbate 80. In some embodiments, at least one elution buffer comprises from about 90 to about 110 mM sodium acetate and from about 0.05 to about 0.2% (w/w) polysorbate 80. In some embodiments, at least one elution buffer comprises about 100 mM sodium acetate and about 0.1% (w/w) polysorbate 80. In some embodiments, the elution buffer has a pH from about 5.0 to about 7.4, about 5.5 to about 7.0, or about 5.5 to about 6.5, or about 6.0. In some embodiments, at least one elution buffer comprises about 100 mM sodium acetate and about 0.1% (w/w) polysorbate 80, and has a pH of about 6.0.

In some embodiments, at least one elution buffer comprises from about 10 to about 200 mM TrisHCl and from about 10 to about 75% (w/w) ethylene glycol. In some embodiments, at least one elution buffer comprises from about 25 mM to about 100 mM TrisHCl and from about 25% to about 70% (w/w) ethylene glycol. In some embodiments, at least one elution buffer comprises from about 40 mM to about 60 mM TrisHCl and from about 40% to about 60% (w/w) ethylene glycol. In some embodiments, at least one elution buffer comprises about 50 mM TrisHCl and about 50% (w/w) ethylene glycol. In some embodiments, the elution buffer has a pH from about 7.5 to about 9.2, about 8.0 to about 9.0, or about 8.0 to about 8.8, or about 8.5. In some embodiments, at least one elution buffer comprises about 50 mM TrisHCl and about 50% (w/w) ethylene glycol, and has a pH of about 8.5.

In some embodiments, at least one elution buffer comprises from about 10 to about 200 mM glycine, about 1 to about 100 mM histidine, about 20 to about 500 mM salt, about 1 to about 10% (w/w) trehalose, and about 0.0005 to about 1% (w/w) polysorbate 80. In some embodiments, at least one elution buffer comprises from about 30 to about 80 mM glycine, about 5 to about 20 mM histidine, about 50 to about 200 mM salt, about 3 to about 8% trehalose, and about 0.001 to about 0.1% (w/w) polysorbate 80. In some embodiments, at least one elution buffer comprises from about 40 to about 60 mM glycine, about 5 to about 15 mM histidine, about 90 to about 110 mM salt, about 4 to about 6% (w/w) trehalose, and about 0.001 to about 0.05% (w/w) polysorbate 80. In some embodiments, at least one elution buffer comprises about 50 mM glycine, about 10 mM histidine, about 100 mM salt, about 5% (w/w) trehalose, and about 0.005% (w/w) polysorbate 80. In some embodiments, the elution buffer has a pH from about 6.0 to about 8.0, about 6.5 to about 7.5, or about 7.0 to about 7.4, or about 7.0. In some embodiments, at least one elution buffer comprises about 50 mM glycine, about 10 mM histidine, about 100 mM salt, about 5% (w/w) trehalose, and about 0.005% (w/w) polysorbate 80, and has a pH of about 7.0.

In some embodiments, at least one elution buffer comprises from about 1 to about 200 mM TrisHCl, from about 50 to about 500 mM salt, and from about 0.001 to about 1% (w/w) polysorbate 80. In some embodiments, at least one elution buffer comprises from about 5 to about 50 mM TrisHCl, from about 75 to about 250 mM salt, and from about 0.005 to about 0.3% (w/w) polysorbate 80. In some embodiments, at least one elution buffer comprises from about 10 to about 30 mM TrisHCl, from about 140 to about 160 mM salt, and from about 0.05% to about 0.2% (w/w) polysorbate 80. In some embodiments, at least one elution buffer comprises about 20 mM TrisHCl, about 150 mM salt, and 0.1% (w/w) polysorbate 80. In some embodiments, the elution buffer has a pH from about 6.0 to about 8.8, about 6.5 to about 8.5, or about 7.0 to about 8.0, or about 7.4. In some embodiments, at least one elution buffer comprises about 20 mM TrisHCl, about 150 mM salt, and 0.1% (w/w) polysorbate 80 and has a pH of about 7.4.

In some embodiments, the first, second, third, and/or fourth wash step comprises applying to the affinity resin a buffer comprising from about 10 to about 200 mM TrisHCl and from about 50 to about 500 mM salt. In some embodiments, the first, second, third, and/or fourth wash step comprises applying to the affinity resin a buffer comprising from about 25 to about 100 mM TrisHCl and from about 75 to about 250 mM salt. In some embodiments, the first, second, third, and/or fourth wash step comprises applying to the affinity resin a buffer comprising from about 40 to about 60 mM TrisHCl and from about 100 to about 150 mM salt. In some embodiments, the first, second, third, and/or fourth wash step comprises applying to the affinity resin a buffer comprising about 50 mM TrisHCl and about 125 mM salt. In some embodiments, the first, second, third, and/or fourth wash step comprises applying to the affinity resin a buffer comprising a pH from about 7.5 to about 9.2, about 8.0 to about 9.0, or about 8.0 to about 8.8, or about 8.5. In some embodiments, the first, second, third, and/or fourth wash step comprises applying to the affinity resin a buffer comprising about 50 mM TrisHCl and about 125 mM salt with a pH of about 8.5.

In some embodiments, the first, second, third, and/or fourth wash step comprises applying to the affinity resin a buffer comprising from about 10 to about 2000 mM sodium acetate and from about 0.001 to about 1% (w/w) polysorbate 80. In some embodiments, the first, second, third, and/or fourth wash step comprises applying to the affinity resin a buffer comprising from about 50 to about 200 mM sodium acetate and about 0.005 to about 0.3% (w/w) polysorbate 80. In some embodiments, the first, second, third, and/or fourth wash step comprises applying to the affinity resin a buffer comprising from about 90 to about 110 mM sodium acetate and about 0.05 to about 0.2% (w/w) polysorbate 80. In some embodiments, the first, second, third, and/or fourth wash step comprises applying to the affinity resin a buffer comprising about 100 mM sodium acetate and about 0.1% (w/w) polysorbate 80. In some embodiments, the first, second, third, and/or fourth wash step comprises a pH from about 5.0 to about 7.4, about 5.5 to about 7.0, or about 5.5 to about 6, or about 6. In some embodiments, the first, second, third, and/or fourth wash step comprises applying to the affinity resin a buffer comprising about 100 mM sodium acetate and about 0.1% (w/w) polysorbate 80 with a pH of about 6.0.

In some embodiments, the first wash step comprises applying to the affinity resin a first buffer comprising from about 10 to about 200 mM TrisHCl and from about 50 to about 500 mM salt. In some embodiments, the first wash step comprises applying to the affinity resin a first buffer comprising from about 25 to about 100 mM TrisHCl and from about 75 to about 250 mM salt. In some embodiments, the first wash step comprises applying to the affinity resin a first buffer comprising from about 40 to about 60 mM TrisHCl and from about 100 to about 150 mM salt. In some embodiments, the first wash step comprises applying to the affinity resin a first buffer comprising about 50 mM TrisHCl and about 125 mM salt. In some embodiments, the first wash step comprises applying to the affinity resin a first buffer comprising a pH from about 7.5 to about 9.2, about 8.0 to about 9.0, or about 8.0 to about 8.8, or about 8.5. In some embodiments, the first wash step comprises applying to the affinity resin a first buffer comprising about 50 mM TrisHCl and about 125 mM salt with a pH of about 8.5.

In some embodiments, the second wash step comprises applying to the affinity resin a second buffer comprising from about 10 to about 2000 mM sodium acetate and from about 0.001 to about 1% (w/w) polysorbate 80. In some embodiments, the second wash step comprises applying to the affinity resin a second buffer comprising from about 50 to about 200 mM sodium acetate and about 0.005 to about 0.3% (w/w) polysorbate 80. In some embodiments, the second wash step comprises applying to the affinity resin a second buffer comprising from about 90 to about 110 mM sodium acetate and about 0.05 to about 0.2% (w/w) polysorbate 80. In some embodiments, the second wash step comprises applying to the affinity resin a second buffer comprising about 100 mM sodium acetate and about 0.1% (w/w) polysorbate 80. In some embodiments, the second wash buffer comprises a pH from about 5.0 to about 7.4, about 5.5 to about 7.0, or about 5.5 to about 6, or about 6. In some embodiments, the second wash step comprises applying to the affinity resin a second buffer comprising about 100 mM sodium acetate and about 0.1% (w/w) polysorbate 80 with a pH of about 6.0.

In some embodiments, the third wash step comprises applying to the affinity resin a third buffer comprising from about 10 to about 200 mM TrisHCl and from about 50 to about 500 mM salt. In some embodiments, the third wash step comprises applying to the affinity resin a third buffer comprising from about 25 to about 100 mM TrisHCl and from about 75 to about 250 mM salt. In some embodiments, the third wash step comprises applying to the affinity resin a third buffer comprising from about 40 to about 60 mM TrisHCl and from about 100 to about 150 mM salt. In some embodiments, the third wash step comprises applying to the affinity resin a third buffer comprising about 50 mM TrisHCl and about 125 mM salt. In some embodiments, the third wash step comprises applying to the affinity resin a third buffer comprising a pH from about 7.5 to about 9.2, about 8.0 to about 9.0, or about 8.0 to about 8.8, or about 8.5. In some embodiments, the third wash step comprises applying to the affinity resin a third buffer comprising about 50 mM TrisHCl and about 125 mM salt with a pH of about 8.5.

In some embodiments, step (c) comprises applying to the affinity resin a buffer comprising about 10 to about 200 mM TrisHCl and from about 50 to about 500 mM salt. In some embodiments, step (c) comprises applying to the affinity resin a buffer comprising about 25 to about 100 mM TrisHCl and from about 75 to about 250 mM salt. In some embodiments, step (c) comprises applying to the affinity resin a buffer comprising about 40 to about 60 mM TrisHCl and from about 100 to about 150 mM salt. In some embodiments, step (c) comprises applying to the affinity resin a buffer comprising about 50 mM TrisHCl and about 125 mM salt. In some embodiments, the elution wash step comprises applying to the affinity resin a buffer comprising a pH from about 7.5 to about 9.2, about 8.0 to about 9.0, or about 8.0 to about 8.8, or about 8.5. In some embodiments, step (c) comprises applying to the affinity resin a buffer comprising about 50 mM TrisHCl and about 125 mM salt with a pH of about 8.5.

In some embodiments, the first wash step comprises applying to the affinity resin a first buffer comprising from about 10 to about 200 mM TrisHCl and from about 50 to about 500 mM salt. In some embodiments, the first wash step comprises applying to the affinity resin a first buffer comprising from about 25 to about 100 mM TrisHCl and from about 75 to about 250 mM salt. In some embodiments, the first wash step comprises applying to the affinity resin a first buffer comprising from about 40 to about 60 mM TrisHCl and from about 100 to about 150 mM salt. In some embodiments, the first wash step comprises applying to the affinity resin a first buffer comprising about 50 mM TrisHCl and about 125 mM salt. In some embodiments, the first wash step comprises applying to the affinity resin a first buffer comprising a pH from about 7.5 to about 9.2, about 8.0 to about 9.0, or about 8.0 to about 8.8, or about 8.5. In some embodiments, the first wash step comprises applying to the affinity resin a first buffer comprising about 50 mM TrisHCl and about 125 mM salt with a pH of about 8.5.

In some embodiments, the second wash step comprises applying to the affinity resin a second buffer comprising from about 10 to about 2000 mM sodium acetate and from about 0.001 to about 1% (w/w) polysorbate 80. In some embodiments, the second wash step comprises applying to the affinity resin a second buffer comprising from about 50 to about 200 mM sodium acetate and about 0.005 to about 0.3% (w/w) polysorbate 80. In some embodiments, the second wash step comprises applying to the affinity resin a second buffer comprising from about 90 to about 110 mM sodium acetate and about 0.05 to about 0.2% (w/w) polysorbate 80. In some embodiments, the second wash step comprises applying to the affinity resin a second buffer comprising about 100 mM sodium acetate and about 0.1% (w/w) polysorbate 80. In some embodiments, the second wash buffer comprises a pH from about 5.0 to about 7.4, about 5.5 to about 7.0, or about 5.5 to about 6, or about 6. In some embodiments, the second wash step comprises applying to the affinity resin a second buffer comprising about 100 mM sodium acetate and about 0.1% (w/w) polysorbate 80 with a pH of about 6.0.

In some embodiments, the third wash step comprises applying to the affinity resin a third buffer comprising from about 10 to about 200 mM TrisHCl and from about 50 to about 500 mM salt. In some embodiments, the third wash step comprises applying to the affinity resin a third buffer comprising from about 25 to about 100 mM TrisHCl and from about 75 to about 250 mM salt. In some embodiments, the third wash step comprises applying to the affinity resin a third buffer comprising from about 40 to about 60 mM TrisHCl and from about 100 to about 150 mM salt. In some embodiments, the third wash step comprises applying to the affinity resin a third buffer comprising about 50 mM TrisHCl and about 125 mM salt. In some embodiments, the third wash step comprises applying to the affinity resin a third buffer comprising a pH from about 7.5 to about 9.2, about 8.0 to about 9.0, or about 8.0 to about 8.8, or about 8.5. In some embodiments, the third wash step comprises applying to the affinity resin a third buffer comprising about 50 mM TrisHCl and about 125 mM salt with a pH of about 8.5.

In some embodiments, the fourth wash step comprises applying to the affinity resin a fourth buffer comprising from about 10 to about 2000 mM sodium acetate and from about 0.001 to about 1% (w/w) polysorbate 80. In some embodiments, the fourth wash step comprises applying to the affinity resin a fourth buffer comprising from about 50 to about 200 mM sodium acetate and about 0.005 to about 0.3% (w/w) polysorbate 80. In some embodiments, the fourth wash step comprises applying to the affinity resin a fourth buffer comprising from about 90 to about 110 mM sodium acetate and about 0.05 to about 0.2% (w/w) polysorbate 80. In some embodiments, the fourth wash step comprises applying to the affinity resin a fourth buffer comprising about 100 mM sodium acetate and about 0.1% (w/w) polysorbate 80. In some embodiments, the fourth wash buffer comprises a pH from about 5.0 to about 7.4, about 5.5 to about 7.0, or about 5.5 to about 6, or about 6. In some embodiments, the fourth wash step comprises applying to the affinity resin a fourth buffer comprising about 100 mM sodium acetate and about 0.1% (w/w) polysorbate 80 with a pH of about 6.0.

In some embodiments, step (c) comprises applying to the affinity resin a buffer comprising about 10 to about 2000 mM sodium acetate and from about 0.001 to about 1% (w/w) polysorbate 80. In some embodiments, step (c) comprises applying to the affinity resin a buffer comprising about 50 to about 200 mM sodium acetate and about 0.005 to about 0.3% (w/w) polysorbate 80. In some embodiments, step (c) comprises applying to the affinity resin a buffer comprising about 90 to about 110 mM sodium acetate and about 0.05 to about 0.2% (w/w) polysorbate 80. In some embodiments, step (c) comprises applying to the affinity resin a buffer comprising about 100 mM sodium acetate and about 0.1% (w/w) polysorbate 80. In some embodiments, the elution buffer comprises a pH from about 5.0 to about 7.4, about 5.5 to about 7.0, or about 5.5 to about 6, or about 6. In some embodiments, step (c) comprises applying to the affinity resin a buffer comprising about 100 mM sodium acetate and about 0.1% (w/w) polysorbate 80 with a pH of about 6.0.

In some embodiments, the first wash step comprises applying to the affinity resin a first buffer comprising from about 10 to about 2000 mM sodium acetate and from about 0.001 to about 1% (w/w) polysorbate 80. In some embodiments, the first wash step comprises applying to the affinity resin a first buffer comprising from about 50 to about 200 mM sodium acetate and about 0.005 to about 0.3% (w/w) polysorbate 80. In some embodiments, the first wash step comprises applying to the affinity resin a first buffer comprising from about 90 to about 110 mM sodium acetate and about 0.05 to about 0.2% (w/w) polysorbate 80. In some embodiments, the first wash step comprises applying to the affinity resin a first buffer comprising about 100 mM sodium acetate and about 0.1% (w/w) polysorbate 80. In some embodiments, the first wash buffer comprises a pH from about 5.0 to about 7.4, about 5.5 to about 7.0, or about 5.5 to about 6, or about 6. In some embodiments, the first wash step comprises applying to the affinity resin a first buffer comprising about 100 mM sodium acetate and about 0.1% (w/w) polysorbate 80 with a pH of about 6.0.

In some embodiments, the second wash step comprises applying to the affinity resin a second buffer comprising from about 10 to about 200 mM TrisHCl and from about 50 to about 500 mM salt. In some embodiments, the second wash step comprises applying to the affinity resin a second buffer comprising from about 25 to about 100 mM TrisHCl and from about 75 to about 250 mM salt. In some embodiments, the second wash step comprises applying to the affinity resin a second buffer comprising from about 40 to about 60 mM TrisHCl and from about 100 to about 150 mM salt. In some embodiments, the second wash step comprises applying to the affinity resin a second buffer comprising about 50 mM TrisHCl and about 125 mM salt. In some embodiments, the second wash step comprises applying to the affinity resin a second buffer comprising a pH from about 7.5 to about 9.2, about 8.0 to about 9.0, or about 8.0 to about 8.8, or about 8.5. In some embodiments, the second wash step comprises applying to the affinity resin a second buffer comprising about 50 mM TrisHCl and about 125 mM salt with a pH of about 8.5.

In some embodiments, the third wash step comprises applying to the affinity resin a third buffer comprising from about 10 to about 2000 mM sodium acetate and from about 0.001 to about 1% (w/w) polysorbate 80. In some embodiments, the third wash step comprises applying to the affinity resin a third buffer comprising from about 50 to about 200 mM sodium acetate and about 0.005 to about 0.3% (w/w) polysorbate 80. In some embodiments, the third wash step comprises applying to the affinity resin a third buffer comprising from about 90 to about 110 mM sodium acetate and about 0.05 to about 0.2% (w/w) polysorbate 80. In some embodiments, the third wash step comprises applying to the affinity resin a third buffer comprising about 100 mM sodium acetate and about 0.1% (w/w) polysorbate 80. In some embodiments, the third wash buffer comprises a pH from about 5.0 to about 7.4, about 5.5 to about 7.0, or about 5.5 to about 6, or about 6. In some embodiments, the third wash step comprises applying to the affinity resin a third buffer comprising about 100 mM sodium acetate and about 0.1% (w/w) polysorbate 80 with a pH of about 6.0.

In some embodiments, step (c) comprises applying to the affinity resin a buffer comprising about 10 to about 2000 mM sodium acetate and from about 0.001 to about 1% (w/w) polysorbate 80. In some embodiments, step (c) comprises applying to the affinity resin a buffer comprising about 50 to about 200 mM sodium acetate and about 0.005 to about 0.3% (w/w) polysorbate 80. In some embodiments, step (c) comprises applying to the affinity resin a buffer comprising about 90 to about 110 mM sodium acetate and about 0.05 to about 0.2% (w/w) polysorbate 80. In some embodiments, step (c) comprises applying to the affinity resin a buffer comprising about 100 mM sodium acetate and about 0.1% (w/w) polysorbate 80. In some embodiments, the elution buffer comprises a pH from about 5.0 to about 7.4, about 5.5 to about 7.0, or about 5.5 to about 6, or about 6. In some embodiments, step (c) comprises applying to the affinity resin a buffer comprising about 100 mM sodium acetate and about 0.1% (w/w) polysorbate 80 with a pH of about 6.0.

In some embodiments, the first wash step comprises applying to the affinity resin a first buffer comprising from about 10 to about 2000 mM sodium acetate and from about 0.001 to about 1% (w/w) polysorbate 80. In some embodiments, the first wash step comprises applying to the affinity resin a first buffer comprising from about 50 to about 200 mM sodium acetate and about 0.005 to about 0.3% (w/w) polysorbate 80. In some embodiments, the first wash step comprises applying to the affinity resin a first buffer comprising from about 90 to about 110 mM sodium acetate and about 0.05 to about 0.2% (w/w) polysorbate 80. In some embodiments, the first wash step comprises applying to the affinity resin a first buffer comprising about 100 mM sodium acetate and about 0.1% (w/w) polysorbate 80. In some embodiments, the first wash buffer comprises a pH from about 5.0 to about 7.4, about 5.5 to about 7.0, or about 5.5 to about 6, or about 6. In some embodiments, the first wash step comprises applying to the affinity resin a first buffer comprising about 100 mM sodium acetate and about 0.1% (w/w) polysorbate 80 with a pH of about 6.0.

In some embodiments, the second wash step comprises applying to the affinity resin a second buffer comprising from about 10 to about 200 mM TrisHCl and from about 50 to about 500 mM salt. In some embodiments, the second wash step comprises applying to the affinity resin a second buffer comprising from about 25 to about 100 mM TrisHCl and from about 75 to about 250 mM salt. In some embodiments, the second wash step comprises applying to the affinity resin a second buffer comprising from about 40 to about 60 mM TrisHCl and from about 100 to about 150 mM salt. In some embodiments, the second wash step comprises applying to the affinity resin a second buffer comprising about 50 mM TrisHCl and about 125 mM salt. In some embodiments, the second wash step comprises applying to the affinity resin a second buffer comprising a pH from about 7.5 to about 9.2, about 8.0 to about 9.0, or about 8.0 to about 8.8, or about 8.5. In some embodiments, the second wash step comprises applying to the affinity resin a second buffer comprising about 50 mM TrisHCl and about 125 mM salt with a pH of about 8.5.

In some embodiments, the third wash step comprises applying to the affinity resin a third buffer comprising from about 10 to about 2000 mM sodium acetate and from about 0.001 to about 1% (w/w) polysorbate 80. In some embodiments, the third wash step comprises applying to the affinity resin a third buffer comprising from about 50 to about 200 mM sodium acetate and about 0.005 to about 0.3% (w/w) polysorbate 80. In some embodiments, the third wash step comprises applying to the affinity resin a third buffer comprising from about 90 to about 110 mM sodium acetate and about 0.05 to about 0.2% (w/w) polysorbate 80. In some embodiments, the third wash step comprises applying to the affinity resin a third buffer comprising about 100 mM sodium acetate and about 0.1% (w/w) polysorbate 80. In some embodiments, the third buffer step comprises a pH from about 5.0 to about 7.4, about 5.5 to about 7.0, or about 5.5 to about 6, or about 6. In some embodiments, the third wash step comprises applying to the affinity resin a third buffer comprising about 100 mM sodium acetate and about 0.1% (w/w) polysorbate 80 with a pH of about 6.0.

In some embodiments, there a fourth wash step comprises applying to the affinity resin a fourth buffer comprising from about 10 to about 200 mM TrisHCl and from about 50 to about 500 mM salt. In some embodiments, the fourth wash step comprises applying to the affinity resin a fourth buffer comprising from about 25 to about 100 mM TrisHCl and from about 75 to about 250 mM salt. In some embodiments, the fourth wash step comprises applying to the affinity resin a fourth buffer comprising from about 40 to about 60 mM TrisHCl and from about 100 to about 150 mM salt. In some embodiments, the fourth wash step comprises applying to the affinity resin a fourth buffer comprising about 50 mM TrisHCl and about 125 mM salt. In some embodiments, the fourth wash step comprises applying to the affinity resin a fourth buffer comprising a pH from about 7.5 to about 9.2, about 8.0 to about 9.0, or about 8.0 to about 8.8, or about 8.5. In some embodiments, the fourth wash step comprises applying to the affinity resin a fourth buffer comprising about 50 mM TrisHCl and about 125 mM salt with a pH of about 8.5.

In some embodiments, step (c) comprises applying to the affinity resin a buffer comprising about 10 to about 200 mM TrisHCl and from about 50 to about 500 mM salt. In some embodiments, step (c) comprises applying to the affinity resin a buffer comprising about 25 to about 100 mM TrisHCl and from about 75 to about 250 mM salt. In some embodiments, step (c) comprises applying to the affinity resin a buffer comprising about 40 to about 60 mM TrisHCl and from about 100 to about 150 mM salt. In some embodiments, step (c) comprises applying to the affinity resin a buffer comprising about 50 mM TrisHCl and about 125 mM salt. In some embodiments, the elution wash step comprises applying to the affinity resin a buffer comprising a pH from about 7.5 to about 9.2, about 8.0 to about 9.0, or about 8.0 to about 8.8, or about 8.5. In some embodiments, step (c) comprises applying to the affinity resin a buffer comprising about 50 mM TrisHCl and about 125 mM salt with a pH of about 8.5.

In some embodiments (e.g., the embodiments above), the salt is selected from NaCl, KCl, MgCl₂, CaCl₂), Sodium Citrate, LiCl, CsCl, Sodium Acetate, and a combination of one or more of NaCl, KCl, MgCl₂, CaCl₂), Sodium Citrate, LiCl, CsCl, and Sodium Acetate. In some embodiments (e.g., the embodiments above), the salt is NaCl.

In some embodiments, the buffer comprises about 50 mM TrisHCl and about 125 mM NaCl with a pH of about 8.5.

In some embodiments, the AAV obtained from the eluting step has a purity level of 99.9% or greater. In some embodiments, the AAV obtained from the eluting step has a purity level of 99.0% or greater.

In some embodiments, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% of the AAV capsids eluted from the elution step (c) are full AAV capsids.

In some embodiments, the affinity resin is AAVx resin.

In some embodiments, the AAV is AAV9. In some embodiments, the AAV9 comprises a peptide comprising the sequence of SEQ ID NO: 1, SEQ ID NO: 2, and/or SEQ ID NO: 3.

In some embodiments, the method further comprises contacting the AAV containing solution with a filter comprising positively charged groups effective to deplete acidic charged contaminants from the AAV containing solution.

In some embodiments, the method further comprises nanofiltration of an AAV fraction to remove viruses greater than 35 nm.

In some embodiments, the method further comprises a polish step comprising performing cation exchange chromatography.

In some embodiments, the method further comprises testing an AAV fraction via an AAV-specific ELISA. In some embodiments, the AAV specific ELISA is a sandwich ELISA specific for AAV.

In another aspect, provided herein an AAV product produced by a method according to any one of the embodiments disclosed herein.

### BRIEF DESCRIPTION OF THE DRAWINGS

FIG. 1 depicts the chromatogram of the separation procedure according to Example 3.

FIG. 2A and FIG. 2B depicts the chromatogram of the separation procedure according to Example 4. Load zone and Wash-Elution zone are separated with “Split screen” function. Blue: UV280 nm, Violet: UV254 nm, Red: Conductivity.

FIG. 3 depicts the chromatogram of the separation procedure according to Example 6.

### DETAILED DESCRIPTION

Provided herein are methods of producing an adeno-associated virus (AAV) product, methods of purifying AAV, and methods of purifying full AAV capsids from a concentrated AAV fraction comprising empty AAV capsids and full AAV capsids.

A feature of AAV vector generation in cell culture is the formation of a complex matrix that comprises material from disrupted cells. In particular, host cell proteins, proteasomes, cell debris and potential virus-specific receptors are often present in the material from disrupted cells. The disclosed methods which include steps for removing host cell material from the final AAV product in conditions that result in greater purity at a physiologically applicable pH.

In one aspect, provided herein is a method of purifying an adeno-associated virus (AAV) comprising

(a) loading an AAV containing solution onto an affinity resin targeted against an epitope on the AAV under conditions that allow binding between the AAV in the solution and the affinity resin;

(b) undertaking at least one wash step at room temperature; and

(c) eluting the AAV from the affinity resin at a temperature of less than 18° C.

In certain aspects, the method comprises eluting AAV capsids from an affinity resin by lowering the buffer temperature from room temperature. In certain embodiments, the AAV can be eluted from the same resin using the same buffer at a lowered temperature of 1 to 12° C. In certain embodiments, the AAV can be eluted from the same resin using the same buffer at a lowered temperature of 2 to 8° C. The increased elution of AAV from lowering the temperature from room temperature is surprising and unexpected. Without being bound to a particular theory, this result was surprising and unexpected because while lower temperatures are known to extend the time for the binding between the antibody and antigen to reach equilibrium, one of skill in the art would not have expected an already bound antigen to be readily released when lowering the temperature below room temperature.

The temperature shift elution protocol has the benefit of a mild elution at a low temperature to help preserve the structure and/or infectivity of the AAV particles. Elution according to the various embodiments described herein can prevent low pH exposure (e.g., elution at near neutral pH) and retain high potency of the AAV. Additionally, use of a mild elution buffer can be easily implemented in a manufacturing environment and is more efficient as there is no need for a buffer change for the elution step. Moreover, the temperature shift elution protocol leads to a higher content of full AAV capsids.

In certain embodiments, AAV9 can bind to a resin described herein at a temperature range of about 20 to 25° C., and can be eluted at a lower temperature of about 1 to 12° C. in the same buffer system. In certain embodiments, AAV9 can bind to a resin described herein at a temperature range of about 20 to 25° C., and can be eluted at a lower temperature of about 2 to 8° C. in the same buffer system. For example, AAV9 can bind to CaptureSelect AAVx resin at room temperature when in a buffer comprising 125 mM NaCl and 50 mM TrisHCl, at pH 8.5 and eluted from the resin using the same buffer at a lower temperature of about 1 to 12° C. or about 2 to 8° C. As another example, AAV9 can bind to CaptureSelect AAVx resin at room temperature when in a buffer comprising 100 mM NaAcetate and 0.1% (w/w) Polysorbate 80, at pH 6.0 and eluted from the resin using the same buffer at a lower temperature of about 1 to 12° C. or about 2 to 8° C.

Further improvement to AAV product purity can be seen when undertaking the wash steps and elution steps in succession according to the various embodiments and examples described herein.

### Definitions

The use of the terms “a,” “an” and “the”, and similar referents in the context of describing the disclosure (especially in the context of the following claims), are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted.

As used herein, the terms “capsid”, “capsid particle”, and “particle” are used interchangeably and refer to an AAV particle composed of at least one intact AAV capsid shell.

As used herein, the term “empty” with regard to AAV or AAV capsids refers to those that lack the complete (i.e., full) gene of interest (GOI). Empty AAV or empty AAV capsids or empty AAV particles are unable to provide a therapeutic benefit. As used herein, the term “full” with regard to AAV or AAV capsids or AAV particles refer to those containing a majority of the complete GOI. Full AAV capsids can provide a therapeutic benefit to recipient patients. In certain embodiments, “full” can also include “incomplete vector DNA” or “truncated vector DNA”. In certain embodiments, “overfilled” with regard to AAV or AAV capsids or AAV particles refers to those containing potentially double packaged or longer genome or GOI DNA (e.g., up to double sized). In certain embodiments, complete versus incomplete and/or truncated and/or overfilled vector DNA can be differentiated with additional analytic methods. Such methods include, without limitation, DNA sizing by capillary electrophoresis, AUC (analytical ultracentrifugation), % Agarose DNA (native or alkaline), gel, southern blot, dot-blot hybridization, UV spectrophotometry, weak anion exchange chromatography, and mass spectrometry (See Resolving Adeno-Associated Viral Particle Diversity with Charge Detection Mass Spectrometry Elizabeth E. Pierson et. al Anal. Chem., 2016, 88 (13), pp 6718-6725, which is incorporated herein in its entirety for all purposes).

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Preferred embodiments of this disclosure are described herein, including the best mode known to the inventors for carrying out the disclosure. Variations of those preferred embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

### Wash and Elution Steps and Buffers

In one aspect, provided herein is a method of purifying an adeno-associated virus (AAV). The method comprises (a) loading an AAV containing solution onto an affinity resin targeted against AAV under conductions that allow binding between the AAV in the solution and the affinity resin; (b) undertaking at least one wash step at room temperature; and (c) eluting the AAV from the affinity resin at a temperature of less than 18° C.

The affinity purification step comprises one or more wash steps. The one or more wash steps can be followed by one or more elution steps. In certain embodiments, the methods of the present disclosure comprise a filtration step, which occurs prior to the affinity purification steps.

In some embodiments, at least two wash steps are performed, each involving the same or different buffer. In some embodiments, at least three wash steps are performed, each involving the same or different buffers. In some embodiments, at least four wash steps are performed, each involving the same or different buffers. In some embodiments, at least 1, 2, 3, 4, 5, 6, 7, 8, 9, 10, 11, 2, 13, 14, 15, 16, 17, 18, 19, or 20 wash steps are performed, each involving the same or different buffers. In some embodiments, two wash steps are performed. In some embodiments, three wash steps are performed. In some embodiments, four wash steps are performed. In certain embodiments, the wash buffers are different. In some embodiments, the wash steps are performed in succession. One or more of these wash steps, or even all of these wash steps, are conducted at room temperature (e.g., between 18-26° C., or 18° C., 18.5° C., 19° C., 19.5° C., 20° C., 20.5° C., 21° C., 21.5° C., 22° C., 22.5° C., 23° C., 23.5° C., 24° C., 24.5° C., 25° C., 25.5° C. or 26° C.). In certain embodiments, all wash steps are performed at room temperature.

In certain embodiments, at least one wash buffer is used. In certain embodiments, at least two different wash buffers may be used. In certain embodiments, at least three different wash buffers may be used. In certain embodiments, at least four different wash buffers may be used. In certain embodiments, one wash buffer may be used. In certain embodiments, two different wash buffers may be used. In certain embodiments, three different wash buffers may be used. In certain embodiments, four different wash buffers may be used.

In certain embodiments, at least one elution step is performed. In certain embodiments, at least two elution steps are performed, each involving the same or different buffer. In certain embodiments, at least three elution steps are performed, each involving the same or different buffers. In some embodiments, at least 1, 2, 3, 4, 5, 6, 7, 8, 9, 10, 11, 2, 13, 14, 15, 16, 17, 18, 19, or 20 elution steps are performed, each involving the same or different buffers. In certain embodiments, one elution step is performed. In certain embodiments, at least two elution steps are performed. In certain embodiments, at least three elution steps are performed. In certain embodiments, at least one elution buffer is the same as at least one of the wash buffer(s). In certain embodiments, at least one elution buffer is different than the wash buffer(s). In certain embodiments, at least one elution buffer is the same as the last wash buffer used in the final wash step before eluting the AAV. In certain embodiments, the first elution buffer is the same as the last wash buffer used in the final wash step before eluting the AAV. Elution is conducted at a temperature of between 1° C. and 12° C. (e.g., between 2° C. and 8° C., or at 1° C., 1.5° C., 2° C., 2.5° C., 3° C., 3.5° C., 4° C., 4.5° C., 5° C., 5.5° C., 6° C., 6.5° C., 7° C., 7.5° C., 8° C., 8.5° C., 9° C., 9.5° C., 10° C., 10.5° C., 11° C., 11.5° C., or 12° C.). Elution according to the various embodiments described herein can prevent low pH exposure (e.g., elution at near neutral pH) and retain high potency of the AAV.

In certain embodiments, at least one elution buffer is used. In certain embodiments, at least two different elution buffers may be used. In certain embodiments, at least three different elution buffers may be used. In certain embodiments, at least four different elution buffers may be used. In certain embodiments, one elution buffer may be used. In certain embodiments, two different elution buffers may be used. In certain embodiments, three different elution buffers may be used. In certain embodiments, four different elution buffers may be used.

In certain embodiments, the shift in temperature below room temperature can occur by a cooling cabinet (e.g., Unichromat 1500), a temperature jacket (e.g., water cooling jacket), and/or use of cold buffers for elution. In certain embodiments, the buffer is made at room temperature before chilling. In certain embodiments, the pH if the buffer is measured at room temperature before chilling.

Various volumes may be used, such as from about 2 column volumes to about 15 column volumes, from about 3 column volumes to about 7 column volumes, from about 4 column volumes to about 8 column volumes, from about 5 column volumes to about 10 column volumes, or from about 7 column volumes to about 12 column volumes. For example, 10 column volumes may be used when the column volume is about 2 ml to about 3 ml. About 5 column volumes, or 5 column volumes, of any wash and/or elution buffers may be used. Alternatively, about 10 column volumes, or 10 column volumes, of any wash and/or elution buffers may be used. Lengthening the time of wash steps may further be undertaken to improve AAV purity.

The wash steps may be effective to remove strongly-bound contaminants from AAV and/or a base resin of the affinity matrix. At the same time, the buffers used in the wash steps do not substantially elute the AAV.

In certain embodiments, at least one wash buffer comprises a chelating agent, e.g., EDTA. In certain embodiments, the wash buffer comprises single amino acids or any combination of two or more amino acids that ensures the pH range and depletion rate of host cell (e.g., HEK)-HCP, for example glycine, arginine, tryptophan, derivatives of amino acids, e.g., taurine (oxidized cysteine), N-Acetyl-Tryptophan, and glycylglycine.

The elution steps may be effective to elutes the AAV capsids. In certain embodiments, the elution steps preferentially elutes full AAV capsids over empty or overfilled AAV capsids. In certain embodiments, the elution buffer comprises single amino acids or any combination of two or more amino acids to ensure pH and elution of AAV, for example glycine, arginine, tryptophan, derivatives of amino acids, e.g., taurine (oxidized cysteine), N-acetyl-tryptophan, and glycylglycine.

Without wishing to be bound by theory, degree of elution of AAV is affected by both the amount of ethylene glycol and the conductivity of salt in the third buffer. An amount of at least 55% (w/w) ethylene glycol in the buffer can significantly increase the amount of elution, as compared to 50% (w/w) ethylene glycol. Accordingly, at a given ethylene glycol concentration, increased NaCl concentration can increase the extent and rate of elution. At a given ethylene glycol concentration, replacement of NaCl with a polyvalent salt also can increase the extent and rate of elution.

Without wishing to be bound by theory, if salt is constant, e.g., 150 mM NaCl, then increasing amount of ethylene glycol can increase the elution strength of the buffer. If the ethylene glycol content is constant, e.g., 55%, then increasing amount of salt can increase the elution strength of the buffer. Thus, the elution strength increases from 40% to 45% to 50% to 55% to 60% (w/w) ethylene glycol in 150 mM NaCl. Increasing the ethylene glycol content of a solution with constant salt content can lower the conductivity. An increased amount of ethylene glycol can lower the amount of solubility of salt in the buffer.

In certain embodiments, one or more of sorbitol, mannitol, xylitol, sucrose, trehalose, glycerol (1,2,3-Propanetriol), or erythritol (meso-1,2,3,4-butantetrol) can be used in conjunction with ethylene glycol or instead of ethylene glycol. In certain embodiments, the elution buffer can comprise from about 30 to about 35%, about 35 to about 40%, about 40 to about 45%, about 45 to about 50%, about 48 to about 52%, about 50 to about 55%, about 55 to about 60%, about 60 to about 65%, about 65 to about 70%, or about 70 to about 75% (w/w) ethylene glycol. In certain embodiments, the elution buffer can comprise about 50%, or 50% (w/w) ethylene glycol. In certain embodiments, the concentration of ethylene glycol is at least 55% (w/w). In certain embodiments, the concentration of ethylene glycol is at least 56% (w/w). In certain embodiments, the concentration of ethylene glycol is at least 57% (w/w). In certain embodiments, the concentration of ethylene glycol is at least 58% (w/w).

For example, the wash and/or elution buffer can comprise one or more of TrisHCl, acetate, phosphate, histidine, imidazole, lysine, arginine, glycine, taurine, citrate, HEPES, MES, MES-Na, borate, Bis-Tris, MOPS, bicine, tricine, TAPS, TAPSO, MES, PIPES, TES (2-[[1,3-dihydroxy-2-(hydroxymethyl)propan-2-yl]amino]ethanesulfonic acid), sodium barbital (Veronal), ADA (N-(2-Acetamido)iminodiacetic acid), ACES (N-(2-Acetamido)-2-aminoethanesulfonic acid), Bis-Tris Propane, BES (N,N-Bis(2-hydroxyethyl)-2-aminoethanesulfonic acid), DIPSO (3-(N,N-Bis[2-hydroxyethyl]amino)-2-hydroxypropanesulfonic acid), Trizma, HEPPSO (4-(2-Hydroxyethyl)piperazine-1-(2-hydroxypropanesulfonic acid)), POPSO (Piperazine-1,4-bis(2-hydroxypropanesulfonic acid) dehydrate), TEA, EPPS (4-(2-Hydroxyethyl)-1-piperazinepropanesulfonic acid), HEPBS (N-(2-Hydroxyethyl)piperazine-N′-(4-butanesulfonic acid), AMPD (2-Amino-2-methyl-1,3-propanediol), AMPSO (N-(1,1-Dimethyl-2-hydroxyethyl)-3-amino-2-hydroxypropanesulfonic acid), single amino acids or any combination of two or more amino acids, for example glycine, arginine, tryptophan, derivatives of amino acids, e.g., taurine (oxidized cysteine), N-Acetyl-Tryptophan, and glycylglycine.

In some embodiments, the wash and/or elution buffer can comprise one or more of sodium acetate, TrisHCl, arginine-HCl, lysine-HCl, and histidine-HCl, histidine, glycine, taurine, MES-Na, Bis-Tris, Citrate, Acetate, MES, HEPES, Phosphate, TrisHCl, Bis-Tris, Histidine, Imidazol, ArgininHCl, LysinHCl, Glycine, Glycylglycine, borate, MOPS, bicine, tricine, TAPS, TAPSO, PIPES, L-Glutamic Acid, Aspartic acid, BAPTA (1,2-bis(o-aminophenoxy)ethane-N,N,N′,N′-tetraacetic acid), and/or N-acetyl-D, L-tryptophan.

In certain embodiments, the wash and/or elution buffer further comprises a salt. In some embodiments, the buffer comprises TrisHCl and a salt. In some embodiments, the buffer comprises Arginine-HCl and a salt. In some embodiments, the buffer comprises histidine and a salt.

For the wash and/or elution buffers described herein, the salt can be selected from NaCl, KCl, MgCl₂, CaCl₂, LiCl, CsCl, sodium acetate (C₂H₃NaO₂), (NH₄)₂SO₄, NH₄Cl, Na₂SO₄, K₂SO₄, sodium citrate, potassium citrate, and a combination of one or more of NaCl, KCl, MgCl₂, CaCl₂, LiCl, CsCl, C₂H₃NaO₂, sodium citrate, and potassium citrate. In some embodiments, the salt concentration is from about 50 to about 2000 mM, about 100 to about 1500 mM, about 100 to about 200 mM, about 200 mM to about 1000 mM, about 500 to about 900 mM, about 600 mM to about 800 mM, about 750 mM, or 750 mM. In some embodiments, the salt concentration is from about 50 to about 2000 mM, about 100 to about 1500 mM, about 100 to about 200 mM, about 200 mM to about 1000 mM, about 500 to about 900 mM, about 600 mM to about 800 mM NaCl, about 750 mM NaCl, or 750 mM NaCl. In some embodiments, when a concentration gradient of NaCl is used, the target concentration is 2000 mM. In some embodiments, the concentration of the salt does not exceed 500 mM. In some embodiments, the concentration of the salt does not exceed 200 mM. In some embodiments, the salt is NaCl. In some embodiments, the salt is 125 mM NaCl. In some embodiments, the salt is 150 mM NaCl.

In certain embodiments, the wash and/or elution buffer can further comprise one or more organic solvent or detergent. For example, the organic solvent or detergent can be, but is not limited to, Tween 80, polysorbate 80, Triton X100, tri (n-butyl) phosphate (TNBP), ethylene glycol, sorbitol, mannitol, xylitol, DMSO, sucrose, or trehalose. For example, the detergent can be, but is not limited to, a nonionic polyoxyethylene surfactant (e.g., Brij 35), 4-Nonylphenyl-polyethylene glycol (Arkopal N100), octylglcoside, n-Dodecyl β-D-maltoside, Digitonin, 6-Cyclohexylhexyl β-D-maltoside, or octylglycopyranoside. For example, ethylene glycol can be PEG, such as but not limited to, PEG 2000, PEG4000, PEG6000 (Macrogol). For example, the organic solvent can be, but not limited to, glycerol (1,2,3-Propanetriol), and erythritol (meso-1,2,3,4-Butantetrol). In some embodiments, the detergent comprises one or more of Triton X100, polysorbate 80, and tri (n-butyl) phosphate (TNBP). In some embodiments, the organic solvent or detergent can be polysorbate 80, ethylene glycol, sorbitol, mannitol, xylitol, sucrose, or trehalose. In some embodiments, the buffer comprises TrisHCl and DMSO.

In certain embodiments, the organic solvent or detergent is present in the wash and/or elution buffer comprising about 0.0005 to about 20%, about 0.0005 to about 15%, about 0.0005 to about 10%, about 0.0005 to about 5%, about 0.0005 to about 1%, about 0.001 to about 4%, about 0.001 to about 0.1%, about 0.001 to about 0.05%, about 0.005 to about 3%, about 0.01 to about 2.5%, about 0.05 to about 5%, about 0.05 to about 2%, 0.05 to about 0.2% or about 0.1 to about 1.5% (w/w). In some embodiments, the organic solvent or detergent is present at about 0.005% (w/w). In some embodiments, the organic solvent or detergent is present at about 0.1% (w/w).

In some embodiments, the organic solvent or detergent is polysorbate 80 (e.g., Tween 80 or Crillet). In some embodiments, the buffer comprises polysorbate 80. In some embodiments, the buffer comprises Arginine-HCl and polysorbate 80. In some embodiments, the buffer comprises Taurine and polysorbate 80. In some embodiments, the buffer comprises TrisHCl and polysorbate 80. In some embodiments, the buffer comprises sodium acetate and polysorbate 80.

In certain embodiments, the wash and/or elution buffer comprises from about 0.0005 to about 20%, about 0.0005 to about 15%, about 0.0005 to about 10%, about 0.0005 to about 5%, about 0.0005 to about 1%, about 0.001 to about 4%, about 0.001 to about 0.1%, about 0.001 to about 0.05%, about 0.005 to about 3%, about 0.01 to about 2.5%, about 0.05 to about 5%, about 0.05 to about 2%, 0.05 to about 0.2% or about 0.1 to about 1.5% (w/w) polysorbate 80. In some embodiments, the wash and/or elution buffer comprises about 0.005% (w/w) polysorbate 80. In some embodiments, the wash and/or elution buffer comprises about 0.1% (w/w) polysorbate 80. In some embodiments, the wash and/or elution buffer comprises about 5% (w/w) polysorbate 80. In some embodiments, the wash and/or elution buffer comprises about 10% (w/w) polysorbate 80. In some embodiments, the wash and/or elution buffer comprises about 20% (w/w) polysorbate 80.

In certain embodiments, the wash and/or elution buffer can comprise from about 30 to about 35%, 35 to about 40%, about 40 to about 45%, about 45 to about 50%, about 48 to about 52%, about 50 to about 55%, about 55 to about 60%, about 60 to about 65%, about 65 to about 70%, or about 70 to about 75% (w/w) ethylene glycol. In certain embodiments, the wash and/or elution buffer can comprise about 50%, or 50% (w/w) ethylene glycol.

The organic solvent or detergent need not be present in all wash and/or elution buffers used. In certain embodiments, an organic solvent or detergent is not present any wash and/or elution buffers used. In certain embodiments, an organic solvent or detergent is present in at least one of the wash buffers used. In certain embodiments, an organic solvent or detergent is present in at least one of the elution buffers used. In some embodiments, a wash buffer, e.g., the first wash buffer, comprises both sodium acetate and polysorbate 80. In some embodiments, a wash buffer, e.g., the second wash buffer, comprises both sodium acetate and polysorbate 80. In some embodiments, a wash buffer comprises one or more of Tween 80, DMSO and tri(n-butyl)phosphate (TNBP). In some embodiments, a wash buffer comprises one or more of Triton-X100, polysorbate 80 and TNBP. In some embodiments, a wash buffer, e.g., the third wash buffer, comprises Tris and ethylene glycol. Without wishing to be bound by theory, the organic solvents and detergents in the wash buffers are effective to remove strongly bound host proteins and virus receptors, while also inactivating and/or disintegrating lipid enveloped viruses.

In some embodiments, the buffer further comprises ethylene glycol, sucrose, taurine, and/or glycerol. In some embodiments, the buffer comprises Arginine-HCl and one of sucrose and glycerol. In some embodiments, the buffer comprises Taurine and ethylene glycol. In some embodiments, the buffer comprises TrisHCl and ethylene glycol. In some embodiments, the buffer comprises sodium acetate and ethylene glycol.

In certain embodiments, the wash and/or elution buffer can be a Tris based buffer comprising a salt (e.g., NaCl). In certain embodiments, the wash and/or elution buffer, which can be a sodium acetate (NaAcetate) based buffer. In certain embodiments, the wash and/or elution buffer can comprise a sodium salt of 2-(N-morpholino)ethanesulfonic acid (MES-Na), EDTA, and a solvent/detergent mixture comprising polysorbate 80, DMSO and tri(n-butyl)phosphate (TNBP). In certain embodiments, the wash and/or elution buffer can comprise from about 50 to about 200 mM taurine, and 0.2 to 1.5% PEG (e.g., PEG 6000). In certain embodiments, the wash and/or elution buffer can comprise Bis-Tris, and a solvent/detergent mixture comprising Triton-X100, polysorbate 80 and TNBP. In certain embodiments, wash and/or elution buffer can comprise a glycine-based buffer, a sodium citrate-based buffer, or an Arginine-HCl based buffer comprising a salt (e.g., NaCl). In certain embodiments, the wash and/or elution buffer can be a Tris-based buffer comprising ethylene glycol and/or NaCl, a taurine-based buffer, or an Arginine-HCl based buffer comprising NaCl. Alternatively, one or more of sorbitol, mannitol, xylitol, sucrose, or trehalose can be used in conjunction with ethylene glycol or instead of ethylene glycol. In certain embodiments, the wash and/or elution buffer can comprise sodium acetate and polysorbate 80.

In certain embodiments, the wash and/or elution buffer comprises from about 10 to about 500 mM of TrisHCl. In certain embodiments, the wash and/or elution buffer comprises from about 10 to about 400 mM, about 10 to about 300 mM, about 10 to about 200 mM, about 15 to about 175 mM, about 20 to about 150 mM, about 25 to about 125 mM, about 25 to about 100 mM, about 30 to about 90 mM, about 35 to about 75 mM or about 40 to about 60 mM TrisHCl. In certain embodiments, the wash and/or elution buffer comprises from about 10 to about 15 mM; about 10 to about 30 mM; about 15 to about 20 mM; about 20 to about 25 mM; about 25 to about 30 mM; about 30 to about 35 mM; about 35 to about 40 mM; about 40 to about 45 mM; about 40 to about 50 mM; about 45 to about 50 mM; about 50 to about 55 mM; about 55 to about 60 mM about 60 to about 65 mM; about 65 to about 70 mM; about 70 to about 75 mM; about 75 to about 80 mM, about 80 to about 90 mM; about 90 to about 100 mM; about 100 to about 110 mM; about 110 to about 120 mM; about 120 to about 130 mM; about 130 to about 140 mM; about 140 to about 150 mM; about 150 to about 160 mM; about 160 to about 170 mM; about 170 to about 180 mM; about 180 to about 190 mM; or about 190 to about 200 mM TrisHCl. In certain embodiments, the wash and/or elution buffer can comprise about 50 mM, or 50 mM TrisHCl. In certain embodiments, the wash and/or elution buffer can comprise about 20 mM, or 20 mM TrisHCl.

In certain embodiments, the wash and/or elution buffer can comprise about 25 mM, about 50 mM, about 75 mM, about 100 mM, about 125 mM, about 150 mM, about 175 mM, about 200 mM, about 225 mM, about 250 mM, about 275 mM, about 300 mM, about 325 mM, about 350 mM, about 375 mM, about 400 mM, about 425 mM about 450 mM, about 475 mM, about 500 mM, about 525 mM, about 550 mM, about 575 mM, about 600 mM, about 625 mM, about 650 mM, about 675 mM, about 700 mM about 725 mM, about 750 mM, about 775 mM, about 800 mM, about 825 mM, about 850 mM, about 875 mM, about 900 mM, about 925 mM, about 950 mM, about 975 mM about 1000 mM, about 1025 mM, about 1050 mM, about 1075 mM, about 1100 mM, about 1125 mM, about 1150 mM, about 1175 mM, about 1200 mM, about 1225 mM, about 1250 mM, about 1275 mM, about 1300 mM, about 1325 mM, about 1350 mM, about 1375 mM, about 1400 mM, about 1425 mM, about 1450 mM, about 1475 mM, about 1500 mM, about 1525 mM, about 1550 mM, about 1575 mM, about 1600 mM, about 1625 mM, about 1650 mM, about 1675 mM, about 1700 mM, about 1725 mM, about 1750 mM, about 1775 mM, about 1800 mM, about 1825 mM, about 1850 mM, about 1875 mM, about 1900 mM, about 1925 mM, about 1950 mM, about 1975 mM, or about 2000 mM TrisHCl. In certain embodiments, the wash and/or elution buffer can comprise about 50 mM, or 50 mM TrisHCl. In certain embodiments, the wash and/or elution buffer can comprise about 20 mM, or 20 mM TrisHCl.

In certain embodiments, the TrisHCl wash and/or elution buffer can further comprise from about 50 to about 500 mM salt. In certain embodiments, the TrisHCl wash and/or elution buffer can further comprise from about 55 to about 400 mM, about 60 to about 350 mM, about 70 to about 300 mM, about 75 to about 250 mM, about 80 to about 200 mM, about 90 to about 175 mM, or about 100 to about 150 mM salt. In certain embodiments, the TrisHCl wash and/or elution buffer can further comprise from about 75 to about 100 mM; about 100 to about 125 mM; about 125 to about 150 mM; about 150 to about 175 mM; about 175 to about 200 mM; about 200 to about 225 mM; or about 225 to about 250 mM salt. In certain embodiments, the TrisHCl wash and/or elution buffer can comprise about 150 mM, or 150 mM salt. In certain embodiments, the TrisHCl wash and/or elution buffer can comprise about 125 mM, or 125 mM salt.

In certain embodiments, the TrisHCl wash and/or elution buffer can further comprise from about 50 to about 500 mM NaCl. In certain embodiments, the TrisHCl wash and/or elution buffer can further comprise from about 55 to about 400 mM, about 60 to about 350 mM, about 70 to about 300 mM, about 75 to about 250 mM, about 80 to about 200 mM, about 90 to about 175 mM, or about 100 to about 150 mM NaCl. In certain embodiments, the TrisHCl wash and/or elution buffer can further comprise from about 75 to about 100 mM; about 100 to about 125 mM; about 125 to about 150 mM; about 150 to about 175 mM; about 175 to about 200 mM; about 200 to about 225 mM; or about 225 to about 250 mM NaCl. In certain embodiments, the TrisHCl wash and/or elution buffer can comprise about 150 mM, or 150 mM NaCl. In certain embodiments, the TrisHCl wash and/or elution buffer can comprise about 125 mM, or 125 mM NaCl.

In certain embodiments, the TrisHCl wash and/or elution buffer can further comprise from about 10 to about 75% (w/w) ethylene glycol. In certain embodiments, the TrisHCl wash and/or elution buffer can further comprise from about 20 to about 72%, about 25% to about 70, about 30 to about 65%, or about 40% to about 60% (w/w) ethylene glycol. In certain embodiments, the TrisHCl wash and/or elution buffer can further comprise from about 30 to about 35%; 35 to about 40%; about 40 to about 45%; about 45 to about 50%; about 48 to about 52%; about 50 to about 55%; about 55 to about 60%; about 60 to about 65%; about 65 to about 70%; or about 70 to about 75% (w/w) ethylene glycol. In certain embodiments, the TrisHCl wash and/or elution buffer can comprise about 50%, or 50% (w/w) ethylene glycol.

In certain embodiments, the TrisHCl wash and/or elution buffer can further comprise an organic solvent or detergent. In some embodiments, the organic solvent or detergent is polysorbate 80 (e.g., Tween 80 or Crillet). In certain embodiments, the polysorbate 80 can be from about 0.0005 to about 5%, about 0.0005 to about 1%, about 0.001 to about 4%, about 0.001 to about 0.1%, about 0.001 to about 0.05%, about 0.005 to about 3%, about 0.01 to about 2.5%, about 0.05 to about 5%, about 0.05 to about 2%, 0.05 to about 0.2% or about 0.1 to about 1.5% (w/w) polysorbate 80. In certain embodiments, the TrisHCl wash and/or elution buffer can comprise about 0.05 to about 0.08%; about 0.08 to about 0.11%; about 0.11 to about 0.14%; about 0.14 to about 0.17%; or about 0.17 to about 0.20% (w/w) polysorbate 80. In certain embodiments, the TrisHCl wash and/or elution buffer can comprise about 0.1%, or 0.1% (w/w) polysorbate 80. In certain embodiments, the TrisHCl wash and/or elution buffer can comprise about 0.005%, or 0.005% (w/w) polysorbate 80. In certain embodiments, the TrisHCl wash and/or elution buffer can comprise about 0.1% (w/w) polysorbate 80.

In certain embodiments, the pH of the TrisHCl wash and/or elution buffer can be from about 7.5 to about 9.2, about 8.0 to about 9.0, or about 8.0 to about 8.8. In certain embodiments, the pH of the TrisHCl wash and/or elution buffer can be from about 7.5 to about 7.7; about 7.7 to about 7.9; about 7.9 to about 8.1; about 8.1 to about 8.3; about 8.3 to about 8.5; about 8.5 to about 8.7; about 8.7 to about 8.9; or about 8.9 to about 9.2. In certain embodiments, the TrisHCl wash and/or elution buffer can have a pH of about 7.4, or 7.4. In certain embodiments, the pH of the TrisHCl wash and/or elution buffer can be about 8.5, or 8.5.

In certain embodiments, the wash and/or elution buffer can comprise from about 10 to about 200 mM TrisHCl and from about 50 to about 500 mM salt. In certain embodiments, the wash and/or elution buffer can comprise from about 25 to about 100 mM TrisHCl and from about 75 to about 250 mM salt. In certain embodiments, the wash and/or elution buffer can comprise from about 40 to about 60 mM TrisHCl and from about 100 to about 150 mM salt. In certain embodiments, the wash and/or elution buffer can comprise about 50 mM TrisHCl and about 125 mM salt. In certain embodiments, the wash and/or elution buffer can comprise 50 mM TrisHCl and 125 mM salt. In certain embodiments, the salt can be NaCl, KCl, MgCl₂, CaCl₂), LiCl, CsCl, sodium acetate (C₂H₃NaO₂), (NH₄)₂SO₄, NH₄Cl, Na₂SO₄, K₂SO₄, sodium citrate, potassium citrate, or a combination thereof. In certain embodiments, the salt is NaCl. In certain embodiments, the wash and/or elution buffer has a pH from about 7.5 to about 9.2, about 8.0 to about 9.0, or about 8.0 to about 8.8. In certain embodiments, the buffer has a pH of about 8.5, or 8.5.

In certain embodiments, the wash and/or elution buffer can comprise from about 10 to about 200 mM TrisHCl and from about 10 to about 75% (w/w) ethylene glycol. In certain embodiments, the wash and/or elution buffer can comprise from about 25 mM to about 100 mM TrisHCl and from about 25% to about 70% (w/w) ethylene glycol. In certain embodiments, the wash and/or elution buffer can comprise from about 40 mM to about 60 mM TrisHCl and from about 40% to about 60% (w/w) ethylene glycol. In certain embodiments, the wash and/or elution buffer can comprise about 50 mM TrisHCl and about 50% (w/w) ethylene glycol. In certain embodiments, the wash and/or elution buffer can comprise 50 mM TrisHCl and 50% (w/w) ethylene glycol. from about 7.5 to about 9.2, about 8.0 to about 9.0, or about 8.0 to about 8.8. In certain embodiments, the wash and/or elution buffer has a pH of about 8.5, or 8.5.

In certain embodiments, the wash and/or elution buffer can comprise from about 1 to about 200 mM TrisHCl, about 50 to about 500 mM salt, and about 0.001 to about 1% (w/w) polysorbate 80. In certain embodiments, the wash and/or elution buffer can comprise from about 5 to about 50 mM TrisHCl, about 75 to about 250 mM salt, and about 0.005 to about 0.3% (w/w) polysorbate 80. In certain embodiments, the wash and/or elution buffer can comprise from 10 mM to about 30 mM TrisHCl, about 140 mM to about 160 mM salt, and about 0.05 to about 0.2% (w/w) polysorbate. In certain embodiments, the wash and/or elution buffer can comprise about 20 mM TrisHCl, about 150 mM salt, and about 0.1% (w/w) polysorbate 80. In certain embodiments, the wash and/or elution buffer can comprise 20 mM TrisHCl, 150 mM salt, and 0.1% (w/w) polysorbate 80. In certain embodiments, the salt can be NaCl, KCl, MgCl₂, CaCl₂), LiCl, CsCl, sodium acetate (C₂H₃NaO₂), (NH₄)₂SO₄, NH₄Cl, Na₂SO₄, K₂SO₄, sodium citrate, potassium citrate, or a combination thereof. In certain embodiments, the salt is NaCl. In certain embodiments, the wash and/or elution buffer can have a pH from about 6.0 to about 8.8, about 6.5 to about 8.5, or about 7.0 to about 8.0. In certain embodiments, the wash and/or elution buffer has a pH of about 7.4, or 7.4.

In certain embodiments, the TrisHCl buffer is the first wash buffer and the elution buffer. In certain embodiments, the TrisHCl buffer is the first and third wash buffer and the elution buffer. In certain embodiments, the TrisHCl buffer is the second wash buffer and elution buffer. In certain embodiments, the TrisHCl buffer is the second and fourth wash buffer and elution buffer. In certain embodiments, the TrisHCl buffer is the second wash buffer but not the elution buffer. In certain embodiments, the TrisHCl buffer is the second and fourth wash but not the elution buffer. About 5 column volumes, or 5 column volumes, of each wash buffer may be used. About 10 column volumes, or 10 column volumes, of each wash buffer may be used. About 5 column volumes, or 5 column volumes, of elution buffer may be used. About 10 column volumes, or 10 column volumes, of elution buffer may be used.

In certain embodiments, the wash and/or elution buffer comprises from about 10 to about 2000 mM sodium acetate. In certain embodiments, the wash and/or elution buffer comprises from about 20 to about 1000 mM, about 30 to about 750 mM, about 40 to about 500 mM, about 50 to about 200 mM, about 75 to about 175 mM, about 80 to about 150 mM, about 85 to about 125 mM, or about 90 to about 110 mM sodium acetate. In certain embodiments, the wash and/or elution buffer can comprise from about 50 to about 75 mM; about 75 to about 100 mM; about 90 to about 110 mM; about 100 to about 125 mM; about 125 to about 150 mM; about 150 to about 175 mM; about 175 to about 200 mM; about 200 to about 250 mM; about 250 to about 300 mM; about 300 to about 350 mM; about 350 to about 400 mM; about 400 to about 450 mM; about 450 to about 500 mM; about 500 to about 550 mM; about 550 to about 600 mM; about 600 to about 650 mM; about 650 to about 700 mM; about 700 to about 750 mM; about 750 to about 800 mM; about 800 to about 850 mM; about 850 to about 900 mM; about 900 to about 950 mM; about 950 to about 1000 mM; about 1000 to about 1050 mM; about 1050 to about 1100 mM; about 1100 to about 1150 mM; about 1150 to about 1200 mM; about 1200 to about 1250 mM; about 1250 to about 1300 mM; about 1300 to about 1350 mM; about 1350 to about 1400 mM; about 1400 to about 1450 mM; about 1450 to about 1500 mM; about 1500 to about 1550 mM; about 1550 to about 1600 mM about 1600 to about 1650 mM; about 1650 to about 1700 mM; about 1700 to about 1750 mM; about 1750 to about 1800 mM; about 1800 to about 1850 mM; about 1850 to about 1900 mM; about 1900 to about 1950 mM; or about 1950 to about 2000 mM sodium acetate. In certain embodiments, the wash and/or elution buffer can comprise about 100 mM, or 100 mM sodium acetate.

In certain embodiments, the wash and/or elution buffer can comprise about 25 mM, about 50 mM, about 75 mM, about 100 mM, about 125 mM, about 150 mM, about 175 mM, about 200 mM, about 225 mM, about 250 mM, about 275 mM, about 300 mM, about 325 mM, about 350 mM, about 375 mM, about 400 mM, about 425 mM about 450 mM, about 475 mM, about 500 mM, about 525 mM, about 550 mM, about 575 mM, about 600 mM, about 625 mM, about 650 mM, about 675 mM, about 700 mM about 725 mM, about 750 mM, about 775 mM, about 800 mM, about 825 mM, about 850 mM, about 875 mM, about 900 mM, about 925 mM, about 950 mM, about 975 mM about 1000 mM, about 1025 mM, about 1050 mM, about 1075 mM, about 1100 mM, about 1125 mM, about 1150 mM, about 1175 mM, about 1200 mM, about 1225 mM, about 1250 mM, about 1275 mM, about 1300 mM, about 1325 mM, about 1350 mM, about 1375 mM, about 1400 mM, about 1425 mM, about 1450 mM, about 1475 mM, about 1500 mM, about 1525 mM, about 1550 mM, about 1575 mM, about 1600 mM, about 1625 mM, about 1650 mM, about 1675 mM, about 1700 mM, about 1725 mM, about 1750 mM, about 1775 mM, about 1800 mM, about 1825 mM, about 1850 mM, about 1875 mM, about 1900 mM, about 1925 mM, about 1950 mM, about 1975 mM, or about 2000 mM sodium acetate. In certain embodiments, the wash and/or elution buffer can comprise about 100 mM, or 100 mM sodium acetate.

In certain embodiments, the wash and/or elution buffer can comprise from about 50 to about 200 mM sodium acetate. In certain embodiments, the wash and/or elution buffer can comprise from about 50 to about 80 mM; about 70 to about 100 mM; about 80 to about 110 mM; about 90 to about 120 mM; about 100 to about 130 mM; about 120 to about 150 mM; about 140 to about 170 mM; about 170 to about 200 mM sodium acetate. In certain embodiments, the wash and/or elution buffer can comprise about 60; about 70 mM; about 80 mM; about 90 mM; about 100 mM; about 110 mM; about 120 mM; about 130 mM; about 140 mM; about 150 mM; or about 160 mM sodium acetate.

In certain embodiments, the sodium acetate wash and/or elution buffer can further comprise an organic solvent or detergent. In some embodiments, the organic solvent or detergent is polysorbate 80 (e.g., Tween 80 or Crillet). In certain embodiments, the polysorbate 80 can be from about 0.0005 to about 5%, about 0.0005 to about 1%, about 0.001 to about 4%, about 0.001 to about 0.1%, about 0.001 to about 0.05%, about 0.005 to about 3%, about 0.01 to about 2.5%, about 0.05 to about 5%, about 0.05 to about 2%, 0.05 to about 0.2% or about 0.1 to about 1.5% (w/w) polysorbate 80. In certain embodiments, the sodium acetate buffer can comprise about 0.05 to about 0.08%; about 0.08 to about 0.11%; about 0.11 to about 0.14%; about 0.14 to about 0.17%; or about 0.17 to about 0.20% (w/w) polysorbate 80. In certain embodiments, the sodium acetate buffer can comprise about 0.1%, or 0.1% (w/w) polysorbate 80. In certain embodiments, the sodium acetate buffer can comprise about 0.005%, or 0.005% (w/w) polysorbate 80. In certain embodiments, the sodium acetate buffer can comprise about 0.1% (w/w) polysorbate 80.

In certain embodiments, the pH of the sodium acetate wash and/or elution buffer can be from about 5.0 to about 7.4, about 5.5 to about 7.0, or about 5.5 to about 6.5. In certain embodiments, the pH of the sodium acetate wash and/or elution buffer can be from about 5.2 to about 5.5; about 5.5 to about 5.8; about 5.8 to about 6.1; about 6.1 to about 6.4; or about 6.4 to about 6.8. In certain embodiments, the sodium acetate wash and/or elution buffer has a pH of about 6.0, or 6.0.

In certain embodiments, the wash and/or elution buffer can comprise from about 10 to about 2000 mM sodium acetate and from about 0.001 to about 1% (w/w) polysorbate 80. In certain embodiments, the wash and/or elution buffer can comprise from about 50 to about 200 mM sodium acetate and from about 0.005 to about 0.3% (w/w) polysorbate 80. In certain embodiments, the wash and/or elution buffer can comprise from about 90 to about 110 mM sodium acetate and from about 0.05 to about 0.2% (w/w) polysorbate 80. In certain embodiments, the wash and/or elution buffer can comprise about 100 mM sodium acetate and about 0.1% (w/w) polysorbate 80. In certain embodiments, the wash and/or elution buffer can comprise 100 mM sodium acetate and 0.1% (w/w) polysorbate 80. In certain embodiments, the wash and/or elution buffer can have a pH from about 5.0 to about 7.4, about 5.5 to about 7.0, or about 5.5 to about 6.5. In certain embodiments, the wash and/or elution buffer has a pH of about 6.0, or 6.0.

In certain embodiments, the sodium acetate buffer is the first wash buffer and the elution buffer. In certain embodiments, the sodium acetate buffer is the first and third wash buffer and the elution buffer. In certain embodiments, the sodium acetate buffer is the second wash buffer and elution buffer. In certain embodiments, the sodium acetate buffer is the second and fourth wash buffer and elution buffer. In certain embodiments, the sodium acetate buffer is the second wash buffer but not the elution buffer. In certain embodiments, the sodium acetate buffer is the second and fourth wash but not the elution buffer.

About 5 column volumes, or 5 column volumes, of each sodium acetate wash buffer may be used. About 10 column volumes, or 10 column volumes, of each sodium acetate wash buffer may be used. About 5 column volumes, or 5 column volumes, of elution sodium acetate buffer may be used. About 10 column volumes, or 10 column volumes, of elution buffer sodium acetate may be used.

In certain embodiments, the wash and/or elution buffer comprises from about 10 to about 500 mM of glycine. In certain embodiments, the wash and/or elution buffer comprises from about 10 to about 400 mM, about 10 to about 300 mM, about 10 to about 200 mM about 15 to about 175 mM, about 20 to about 150 mM, about 25 to about 125 mM, about 25 to about 100 mM, about 30 to about 90 mM, about 35 to about 75 mM or about 40 to about 60 mM glycine. In certain embodiments, the wash and/or elution buffer comprises from about 30 to about 35 mM; about 35 to about 40 mM; about 40 to about 45 mM; about 45 to about 50 mM; about 50 to about 55 mM; about 55 to about 60 mM; about 60 to about 65 mM; about 65 to about 70 mM; about 70 to about 75 mM; about 75 to about 80 mM, about 80 to about 90 mM; about 90 to about 100 mM; about 100 to about 110 mM; about 110 to about 120 mM; about 120 to about 130 mM; about 130 to about 140 mM; about 140 to about 150 mM; about 150 to about 160 mM; about 160 to about 170 mM; about 170 to about 180 mM; about 180 to about 190 mM; or about 190 to about 200 mM glycine. In certain embodiments, the wash and/or elution buffer can comprise about 50 mM, or 50 mM glycine.

In certain embodiments, the wash and/or elution buffer can comprise about 25 mM, about 50 mM, about 75 mM, about 100 mM, about 125 mM, about 150 mM, about 175 mM, about 200 mM, about 225 mM, about 250 mM, about 275 mM, about 300 mM, about 325 mM, about 350 mM, about 375 mM, about 400 mM, about 425 mM about 450 mM, about 475 mM, about 500 mM, about 525 mM, about 550 mM, about 575 mM, about 600 mM, about 625 mM, about 650 mM, about 675 mM, about 700 mM about 725 mM, about 750 mM, about 775 mM, about 800 mM, about 825 mM, about 850 mM, about 875 mM, about 900 mM, about 925 mM, about 950 mM, about 975 mM about 1000 mM, about 1025 mM, about 1050 mM, about 1075 mM, about 1100 mM, about 1125 mM, about 1150 mM, about 1175 mM, about 1200 mM, about 1225 mM, about 1250 mM, about 1275 mM, about 1300 mM, about 1325 mM, about 1350 mM, about 1375 mM, about 1400 mM, about 1425 mM, about 1450 mM, about 1475 mM, about 1500 mM, about 1525 mM, about 1550 mM, about 1575 mM, about 1600 mM, about 1625 mM, about 1650 mM, about 1675 mM, about 1700 mM, about 1725 mM, about 1750 mM, about 1775 mM, about 1800 mM, about 1825 mM, about 1850 mM, about 1875 mM, about 1900 mM, about 1925 mM, about 1950 mM, about 1975 mM, or about 2000 mM glycine. In certain embodiments, the wash and/or elution buffer can comprise about 50 mM, or 50 mM glycine.

In certain embodiments, the wash and/or elution buffer comprises from about 1 to about 300 mM of histidine. In certain embodiments, the wash and/or elution buffer comprises from about 1 to about 250 mM, about 1 to about 200 mM, about 1 to about 100 mM about 1.5 to about 175 mM, about 2.0 to about 150 mM, about 2.5 to about 125 mM, about 2.5 to about 100 mM, about 3.0 to about 90 mM, about 3.5 to about 75 mM or about 4.0 to about 60 mM, about 5.0 to about 50 mM, about 6.0 to about 40 mM, about 7.0 to about 30 mM, about 8.0 to about 20 mM, about 9.0 to about 15 mM histidine. In certain embodiments, the wash and/or elution buffer comprises from about 3.0 to about 3.5 mM; about 3.5 to about 4.0 mM; about 4.0 to about 4.5 mM; about 4.5 to about 5.0 mM; about 5.0 to about 5.5 mM; about 5.5 to about 6.0 mM; about 6.0 to about 6.5 mM; about 6.5 to about 7.0 mM; about 7.0 to about 7.5 mM; about 7.5 to about 8.0 mM, about 8.0 to about 9.0 mM; about 9.0 to about 10.0 mM; about 10.0 to about 11.0 mM; about 11.0 to about 12.0 mM; about 12.0 to about 13.0 mM; about 13.0 to about 14.0 mM; about 14.0 to about 15.0 mM; about 15.0 to about 16.0 mM; about 16.0 to about 17.0 mM; about 17.0 to about 18.0 mM; about 18.0 to about 19.0 mM; or about 19.0 to about 20.0 mM histidine. In certain embodiments, the wash and/or elution buffer can comprise about 10 mM, or 10 mM histidine.

In certain embodiments, the wash and/or elution buffer can comprise about 2.5 mM, about 5.0 mM, about 7.5 mM, about 10.0 mM, about 12.5 mM, about 15.0 mM about 17.5 mM, about 20.0 mM, about 22.5 mM, about 25.0 mM, about 27.5 mM, about 30.0 mM, about 32.5 mM, about 35.0 mM, about 37.5 mM, about 40.0 mM, about 42.5 mM, about 45.0 mM, about 47.5 mM, about 50.0 mM, about 52.5 mM, about 55.0 mM, about 57.5 mM, about 60.0 mM, about 62.5 mM, about 65.0 mM, about 67.5 mM, about 70.0 mM, about 72.5 mM, about 75.0 mM, about 77.5 mM, about 80.0 mM, about 82.5 mM, about 85.0 mM, about 87.5 mM, about 90.0 mM, about 92.5 mM, about 95.0 mM, about 97.5 mM, about 100.0 mM, about 102.5 mM, about 105.0 mM, about 107.5 mM, about 110.0 mM, about 112.5 mM, about 115.0 mM, about 117.5 mM, about 120.0 mM about 122.5 mM, about 125.0 mM, about 127.5 mM, about 130.0 mM, about 132.5 mM about 135.0 mM, about 137.5 mM, about 140.0 mM, about 142.5 mM, about 145.0 mM about 147.5 mM, about 150.0 mM, about 152.5 mM, about 155.0 mM, about 157.5 mM about 160.0 mM, about 162.5 mM, about 165.0 mM, about 167.5 mM, about 170.0 mM about 172.5 mM, about 175.0 mM, about 177.5 mM, about 180.0 mM, about 182.5 mM about 185.0 mM, about 187.5 mM, about 190.0 mM, about 192.5 mM, about 195.0 mM about 197.5 mM, or about 200.0 mM histidine. In certain embodiments, the wash and/or elution buffer can comprise about 10 mM, or 10 mM histidine.

In certain embodiments, the wash and/or elution buffer can further comprise from about 1 to about 75% (w/w) trehalose. In certain embodiments, the wash and/or elution buffer can further comprise from about 2 to about 50%, about 2.5% to about 25%, about 3.0 to about 20%, or about 4.0% to about 10% (w/w) trehalose. In certain embodiments, the wash and/or elution buffer can further comprise from about 3.0 to about 3.5%; 3.5 to about 4.0%; about 4.0 to about 4.5%; about 4.5 to about 5.0%; about 4.8 to about 5.2%; about 5.0 to about 5.5%; about 5.5 to about 6.0%; about 6.0 to about 6.5%; about 6.5 to about 7.0%; or about 7.0 to about 7.5%; about 7.5 to about 8.0%; about 8.0 to about 8.5%; about 8.5 to about 9.0%; about 9.0 to about 9.5%; about 9.5 to about 10%; about 10 to about 15%; about 15 to about 20%; about 20 to about 25%; about 25 to about 30%; about 30 to about 35%; about 35 to about 40%; about 40 to about 45%; about 45 to about 50%; about 50 to about 55%; about 55 to about 60%; about 60 to about 65%; about 65 to about 70%; about 70 to about 75% (w/w) trehalose. In certain embodiments, the wash and/or elution buffer can comprise about 5.0%, or 5.0% (w/w) trehalose.

In certain embodiments, the wash and/or elution buffer can further comprise an organic solvent or detergent. In some embodiments, the organic solvent or detergent is polysorbate 80 (e.g., Tween 80 or Crillet). In certain embodiments, the polysorbate 80 can be from about 0.0005 to about 5%, about 0.0005 to about 1%, about 0.0006 to about 4%, about 0.0007 to about 0.1%, about 0.0008 to about 0.05%, about 0.0009 to about 3%, about 0.001 to about 2.5%, about 0.002 to about 5%, about 0.003 to about 2%, or 0.004 to about 0.2% (w/w) polysorbate 80. In certain embodiments, the wash and/or elution buffer can comprise about 0.05 to about 0.08%; about 0.08 to about 0.11%; about 0.11 to about 0.14%; about 0.14 to about 0.17%; or about 0.17 to about 0.20% (w/w) polysorbate 80. In certain embodiments, the wash and/or elution buffer can comprise about 0.1%, or 0.1% (w/w) polysorbate 80. In some embodiments, the polysorbate 80 can comprise about 0.005%, or 0.005% (w/w) polysorbate 80. In some embodiments, the polysorbate 80 is 0.005%.

In certain embodiments, the pH of the wash and/or elution buffer can be from about 5.0 to about 9.0, about 5.5 to about 8.0, or about 6.0 to about 7.5. In certain embodiments, the pH of the wash and/or elution buffer can be from about 5.2 to about 5.5; about 5.5 to about 5.8; about 5.8 to about 6.1; about 6.1 to about 6.4; about 6.4 to about 6.8; about 6.8 to about 7.0; about 7.0 to about 7.2; about 7.2 to about 7.4; about 7.4 to about 7.8; about 7.8 to about 8.0; about 8.0 to about 8.2; about 8.2 to about 8.4; about 8.4 to about 8.6; about 8.6 to about 8.8; or about 8.8 to about 9.0. In certain embodiments, the wash and/or elution buffer has a pH of about 7.0, or 7.0.

In certain embodiments, the wash and/or elution buffer can comprise from about 10 to about 200 mM glycine, about 1 to about 100 mM histidine, about 20 to about 500 mM salt, about 1 to about 10% trehalose, and about 0.0005 to about 1% (w/w) polysorbate 80. In certain embodiments, the wash and/or elution buffer can comprise from about 30 to about 80 mM glycine, about 5 to about 20 mM histidine, about 50 to about 200 mM salt, about 3 to about 8% trehalose, and about 0.001 to about 0.1% (w/w) polysorbate 80. In certain embodiments, the wash and/or elution buffer can comprise from about 40 to about 60 mM glycine, about 5 to about 15 mM histidine, about 90 to about 110 mM salt, about 4 to about 6% trehalose, and about 0.001 to about 0.05% (w/w) polysorbate 80. In certain embodiments, the wash and/or elution buffer can comprise about 50 mM glycine, about 10 mM histidine, about 100 mM salt, about 5% trehalose, and about 0.005% (w/w) polysorbate 80. In certain embodiments, the wash and/or elution buffer can comprise 50 mM glycine, 10 mM histidine, 100 mM salt, 5% trehalose, and 0.005% (w/w) polysorbate 80. In certain embodiments, the salt can be NaCl, KCl, MgCl₂, CaCl₂), LiCl, CsCl, sodium acetate (C₂H₃NaO₂), (NH₄)₂SO₄, NH₄Cl, Na₂SO₄, K₂SO₄, sodium citrate, potassium citrate, or a combination thereof. In certain embodiments, the salt is NaCl. In certain embodiments, the buffer has a pH from about 6.0 to about 8.0, about 6.5 to about 7.5, or about 7.0 to about 7.4. In certain embodiments, the buffer has a pH of about 7.0 to about 7.4.

In certain embodiments, the wash and/or elution buffer can comprise from about 10 to about 200 mM glycine, about 1 to about 100 mM histidine, about 20 to about 500 mM NaCl, about 1 to about 10% trehalose, and about 0.0005 to about 1% (w/w) polysorbate 80. In certain embodiments, the wash and/or elution buffer can comprise from about 30 to about 80 mM glycine, about 5 to about 20 mM histidine, about 50 to about 200 mM NaCl, about 3 to about 8% trehalose, and about 0.001 to about 0.1% (w/w) polysorbate 80. In certain embodiments, the wash and/or elution buffer can comprise from about 40 to about 60 mM glycine, about 5 to about 15 mM histidine, about 90 to about 110 mM NaCl, about 4 to about 6% trehalose, and about 0.001 to about 0.05% (w/w) polysorbate 80. In certain embodiments, the wash and/or elution buffer can comprise about 50 mM glycine, about 10 mM histidine, about 100 mM NaCl, about 5% trehalose, and about 0.005% (w/w) polysorbate 80. In certain embodiments, the wash and/or elution buffer can comprise 50 mM glycine, 10 mM histidine, 100 mM NaCl, 5% trehalose, and 0.005% (w/w) polysorbate 80. 7.5, or about 7.0 to about 7.4. In certain embodiments, the buffer has a pH of about 7.0 to about 7.4.

In certain embodiments, the glycine buffer is the first wash buffer and the elution buffer. In certain embodiments, the glycine buffer is the first and third wash buffer and the elution buffer. In certain embodiments, the glycine buffer is the second wash buffer and elution buffer. In certain embodiments, the glycine buffer is the second and fourth wash buffer and elution buffer. In certain embodiments, the glycine buffer is the second wash buffer but not the elution buffer. In certain embodiments, the glycine buffer is the second and fourth wash but not the elution buffer.

About 5 column volumes, or 5 column volumes, of each glycine wash buffer may be used. About 10 column volumes, or 10 column volumes, of each glycine wash buffer may be used. About 5 column volumes, or 5 column volumes, of glycine elution buffer may be used. About 10 column volumes, or 10 column volumes, of glycine elution buffer may be used.

In certain embodiments, the wash and/or elution buffer can comprise from about 50 to about 500 mM sodium salt of 2-(N-morpholino)ethanesulfonic acid (MES-Na), from about 3 to about 30 mM EDTA, and a solvent/detergent mixture comprising polysorbate 80, DMSO and tri(n-butyl)phosphate (TNBP). In certain embodiments, the wash and/or elution buffer can comprise from about 50 to about 75 mM; about 75 to about 100 mM; about 90 to about 110 mM; about 100 to about 125 mM; about 125 to about 150 mM; about 150 to about 175 mM; about 175 to about 200 mM; about 200 to about 250 mM; about 250 to about 300 mM; about 300 to about 350 mM; about 350 to about 400 mM; about 400 to about 450 mM; or about 450 to about 500 mM sodium salt of MES-Na. In certain embodiments, the wash and/or elution buffer can comprise about 50; about 75; about 90 mM; about 100 mM; about 125 mM; about 150 mM; about 175 mM; about 200 mM; about 250 mM; about 300 mM; about 350 mM; about 400 mM; about 450 mM; or about 500 mM sodium salt of MES-Na.

In certain embodiments, the wash and/or elution buffer can comprise from about 50 to about 200 mM taurine. In certain embodiments, the wash and/or elution buffer can comprise from about 50 to about 75 mM; about 75 to about 100 mM; about 90 to about 110 mM; about 100 to about 125 mM; about 125 to about 150 mM; about 150 to about 175 mM; about 175 to about 200 mM taurine. In certain embodiments, the wash and/or elution buffer can comprise about 50; about 75; about 90 mM; about 100 mM; about 125 mM; about 150 mM; about 175 mM; about 200 mM taurine.

In certain embodiments, the wash and/or elution buffer can comprise from about 80 to about 400 mM Bis-Tris. In certain embodiments, the wash and/or elution buffer can comprise from about 80 to about 100 mM; about 90 to about 110 mM; about 100 to about 125 mM; about 125 to about 150 mM; about 150 to about 175 mM; about 175 to about 200 mM; about 200 to about 250 mM; about 250 to about 300 mM; about 300 to about 350 mM; about 350 to about 400 mM Bis-Tris. In certain embodiments, the wash and/or elution buffer can comprise about 50; about 75; about 90 mM; about 100 mM; about 125 mM; about 150 mM; about 175 mM; about 200 mM; about 250 mM about 300 mM; about 350 mM; about 400 mM Bis-Tris.

The wash and/or elution buffer can comprise from about 30 to about 35 mM; about 35 to about 40 mM; about 40 to about 45 mM; about 45 to about 50 mM; about 50 to about 55 mM; about 55 to about 60 mM; about 60 to about 65 mM; about 65 to about 70 mM; about 70 to about 75 mM; or about 75 to about 80 mM Arginine-HCl. In certain embodiments, the wash and/or elution buffer can comprise about 50 mM, or 50 mM Arginine-HCl. In certain embodiments, the wash and/or elution buffer can comprise from about 75 to about 100 mM; about 100 to about 125 mM; about 125 to about 150 mM; about 150 to about 175 mM; about 175 to about 200 mM; about 200 to about 225 mM; or about 225 to about 250 mM NaCl. In certain embodiments, the wash and/or elution buffer can comprise about 150 mM, or 150 mM NaCl. In certain embodiments, the pH of the second buffer can be from about 7.5 to about 7.7; about 7.7 to about 7.9; about 7.9 to about 8.1; about 8.1 to about 8.3; about 8.3 to about 8.5; about 8.5 to about 8.7; about 8.7 to about 8.9; or about 8.9 to about 9.2. In certain embodiments, the pH of the wash and/or elution buffer can be about 8.5, or 8.5.

The wash and/or elution buffer can comprise from about 50 to about 200 mM glycine. In certain embodiments, the wash and/or elution buffer can comprise from about 50 to about 100 mM; about 70 to about 120 mM; about 100 to about 150 mM; about 120 to about 170 mM; about 150 to about 200 mM glycine. In certain embodiments, the pH of the wash and/or elution buffer can be from about 7.5 to about 7.7; about 7.7 to about 7.9; about 7.9 to about 8.1; about 8.1 to about 8.3; about 8.3 to about 8.5; about 8.5 to about 8.7; about 8.7 to about 8.9; or about 8.9 to about 9.2. In certain embodiments, the pH of the wash and/or elution buffer can be about 8.5, or 8.5.

The wash and/or elution buffer can comprise from about 50 to about 20 mM sodium citrate. In certain embodiments, the second buffer can comprise from about 5 to about 10 mM; about 7 to about 12 mM; about 10 to about 15 mM; about 12 to about 17 mM; about 15 to about 20 mM sodium citrate. In certain embodiments, the pH of the wash and/or elution buffer can be from about 7.5 to about 7.7; about 7.7 to about 7.9; about 7.9 to about 8.1; about 8.1 to about 8.3; about 8.3 to about 8.5; about 8.5 to about 8.7; about 8.7 to about 8.9; or about 8.9 to about 9.2. In certain embodiments, the pH of the wash and/or elution buffer can be about 8.5, or 8.5.

The wash and/or elution buffer can comprise from about 20 to about 100 mM Histidine and from about 75 to about 250 mM NaCl, with a pH from about 7.5 to about 8.8. In certain embodiments, the wash and/or elution buffer can comprise from about 20 to about 40 mM; about 40 to about 60 mM; about 60 to about 75 mM; or about 75 to about 100 mM Histidine. In certain embodiments, the wash and/or elution buffer can comprise about 20 mM, or 20 mM Histidine. In certain embodiments, the wash and/or elution buffer can comprise from about 75 to about 100 mM; about 100 to about 125 mM; about 125 to about 150 mM; about 150 to about 175 mM; about 175 to about 200 mM; about 200 to about 225 mM NaCl; or about 225 to about 250 mM NaCl. In certain embodiments, the wash and/or elution buffer can comprise about 150 mM, or 150 mM NaCl. In certain embodiments, the wash and/or elution buffer can have a pH may be from about 7.5 to about 7.9; about 7.8 to about 8.2; about 8.1 to about 8.5; about 8.4 to about 8.9; or about 8.6 to about 9.0. In certain embodiments, the wash and/or elution buffer can have a pH of about 8.0, or 8.0.

The wash and/or elution buffer can comprise from about 30 to about 200 mM TrisHCl and from about 30 to about 75% (w/w) ethylene glycol, with a pH from about 7.5 to about 9.2. The wash and/or elution buffer can comprise from about 20 to about 80 mM Arginine-HCl and from about 50 to about 200 mM salt, with a pH from about 7.3 to about 8.8. The wash and/or elution buffer can comprise about 50 mM TrisHCl and about 50% (w/w) ethylene glycol, with a pH of about 8.5. The wash and/or elution buffer can comprise about 20 to about 150 mM taurine, about 30 to about 75% (w/w) ethylene glycol, and from 0.05 to 0.2% (w/w) octylglycopyranoside, with a pH from about 7.3 to about 8.8. The wash and/or elution buffer can comprise about 50 to about 200 mM Arginine-HCl, about 50 to about 200 mM Lysine HCl, about 50 to about 200 mM Histidine-HCl, and about 1 mM to about 4 mM N-acetyl-D,L-tryptophan, and about 10% to about 40% (w/w) polysorbate 80, with a pH from about 7.3 to about 8.8. In certain embodiments, if a salt, e.g., NaCl, is present in the wash and/or elution buffer, in certain embodiments the concentration of the salt does not exceed 500 mM and in certain embodiments, the concentration of the salt does not exceed 200 mM. In certain embodiments, the salt is NaCl, KCl, MgCl₂, CaCl₂, sodium citrate, LiCl, CsCl, sodium acetate, or a combination of one or more of NaCl, KCl, MgCl₂, CaCl₂, sodium citrate, LiCl, CsCl, and sodium acetate. In certain embodiments, the salt is NaCl. In certain embodiments, one or more of sorbitol, mannitol, xylitol, sucrose, or trehalose can be used in conjunction with ethylene glycol or instead of ethylene glycol.

In certain embodiments, the first wash step uses a first buffer, which can be a TrisHCl based buffer. In certain embodiments, the first wash step uses a first buffer, which can be a sodium acetate (NaAcetate) based buffer. In certain embodiments, the first wash step uses a first buffer comprising a sodium salt of 2-(N-morpholino)ethanesulfonic acid (MES-Na), EDTA, and a solvent/detergent mixture comprising polysorbate 80, DMSO and tri(n-butyl)phosphate (TNBP). In certain embodiments, the first wash step uses a first buffer comprising from about 50 to about 200 mM taurine, and 0.2 to 1.5% PEG (e.g., PEG 6000). In certain embodiments, the first wash step uses a first buffer comprising Bis-Tris, and a solvent/detergent mixture comprising Triton-X100, polysorbate 80 and TNBP. In certain embodiments, the first wash step uses a first buffer comprising sodium acetate and polysorbate 80. One or more of these wash steps, or even all of these wash steps, are conducted at room temperature. In certain embodiments, the first wash step uses a first buffer, which can be a TrisHCl based buffer comprising NaCl.

In certain embodiments, the second wash step uses a second buffer, which can be a TrisHCl based buffer. In certain embodiments, the second wash step uses a second buffer, which can be a sodium acetate (NaAcetate) based buffer. In certain embodiments, the second wash step uses a second buffer comprising a sodium salt of 2-(N-morpholino)ethanesulfonic acid (MES-Na), EDTA, and a solvent/detergent mixture comprising polysorbate 80, DMSO and tri(n-butyl)phosphate (TNBP). In certain embodiments, the second wash step uses a second buffer comprising from about 50 to about 200 mM taurine, and 0.2 to 1.5% PEG (e.g., PEG 6000). In certain embodiments, the second wash step uses a second buffer comprising Bis-Tris, and a solvent/detergent mixture comprising Triton-X100, polysorbate 80 and TNBP. In certain embodiments, the second wash step uses a second buffer comprising sodium acetate and polysorbate 80. In certain embodiments, the second wash step uses a second buffer comprising TrisHCl and NaCl buffer.

In certain embodiments, at least one wash buffer may comprise from about 10 to about 200 mM TrisHCl and from about 50 to about 500 mM salt with a pH of about 7.5 to about 9.2. In certain embodiments, at least one wash buffer may comprise from about 25 to about 100 mM TrisHCl and from about 75 to about 250 mM salt with a pH of about 8.0 to about 9.0. In certain embodiments, at least one wash buffer may comprise from about 40 to about 60 mM TrisHCl and from about 100 to about 150 mM salt with a pH of about 8.0 to about 9.0. In certain embodiments, at least one wash buffer may comprise or about 50 mM TrisHCl and about 125 mM salt with a pH of about 8.5. In certain embodiments, the salt can be NaCl, KCl, MgCl₂, CaCl₂), LiCl, CsCl, sodium acetate (C₂H₃NaO₂), (NH₄)₂SO₄, NH₄Cl, Na₂SO₄, K₂SO₄, sodium citrate, potassium citrate, or a combination thereof. In certain embodiments, the salt is NaCl. One or more of these wash steps, or even all of these wash steps, are conducted at room temperature. In certain embodiments, the wash steps occur at room temperature.

In certain embodiments, at least one wash buffer may comprise from about 10 to about 2000 mM sodium acetate and about 0.001 to about 1% (w/w) polysorbate 80 with a pH of about 5.0 to about 7.4. In certain embodiments, at least one wash buffer may comprise from about 50 to about 200 mM sodium acetate and about 0.005 to about 0.3% (w/w) polysorbate 80 with a pH of about 5.5 to about 7.0. In certain embodiments, at least one wash buffer may comprise from about 50 to about 200 mM sodium acetate and about 0.05 to about 0.2% (w/w) polysorbate 80 with a pH of about 5.5 to about 6.5. In certain embodiments, at least one wash buffer may comprise of about 100 mM sodium acetate and about 0.1% (w/w) polysorbate 80 with a pH of about 6.0. One or more of these wash steps, or even all of these wash steps, are conducted at room temperature. In certain embodiments, the wash steps occur at room temperature.

In certain embodiments, at least two wash buffers are used. In certain embodiments, at least one wash buffer may comprise from about 10 to about 200 mM TrisHCl and from about 50 to about 500 mM salt with a pH of about 7.5 to about 9.2. In certain embodiments, at least one wash buffer may comprise from about 10 to about 2000 mM sodium acetate and about 0.001 to about 1% (w/w) polysorbate 80 with a pH of about 5.0 to about 7.4. In certain embodiments, the salt can be NaCl, KCl, MgCl₂, CaCl₂), LiCl, CsCl, sodium acetate (C₂H₃NaO₂), (NH₄)₂SO₄, NH₄Cl, Na₂SO₄, K₂SO₄, sodium citrate, potassium citrate, or a combination thereof. One or more of these wash steps, or even all of these wash steps, are conducted at room temperature. In certain embodiments, the salt is NaCl. In certain embodiments, the wash steps occur at room temperature.

In certain embodiments, at least two wash buffers are used. In certain embodiments, at least one wash buffer may comprise from about 25 to about 100 mM TrisHCl and from about 75 to about 250 mM salt with a pH of about 8.0 to about 9.0. In certain embodiments, at least one wash buffer may comprise from about 50 to about 200 mM sodium acetate and about 0.005 to about 0.3% (w/w) polysorbate 80 with a pH of about 5.5 to about 7.0. In certain embodiments, the salt can be NaCl, KCl, MgCl₂, CaCl₂), LiCl, CsCl, sodium acetate (C₂H₃NaO₂), (NH₄)₂SO₄, NH₄Cl, Na₂SO₄, K₂SO₄, sodium citrate, potassium citrate, or a combination thereof. One or more of these wash steps, or even all of these wash steps, are conducted at room temperature. In certain embodiments, the salt is NaCl. In certain embodiments, the wash steps occur at room temperature.

In certain embodiments, at least two wash buffers are used. In certain embodiments, at least one wash buffer may comprise from about 40 to about 60 mM TrisHCl and from about 100 to about 150 mM salt with a pH of about 8.0 to about 9.0. In certain embodiments, at least one wash buffer may comprise from about 90 to about 100 mM sodium acetate and about 0.05 to about 0.2% (w/w) polysorbate 80 with a pH of about 5.5 to about 6.5. In certain embodiments, the salt can be NaCl, KCl, MgCl₂, CaCl₂), LiCl, CsCl, sodium acetate (C₂H₃NaO₂), (NH₄)₂SO₄, NH₄Cl, Na₂SO₄, K₂SO₄, sodium citrate, potassium citrate, or a combination thereof. One or more of these wash steps, or even all of these wash steps, are conducted at room temperature. In certain embodiments, the salt is NaCl. In certain embodiments, the wash steps occur at room temperature.

In certain embodiments, at least two wash buffers are used. In certain embodiments, at least one wash buffer may comprise or about 50 mM TrisHCl and about 125 mM salt with a pH of about 8.5. In certain embodiments, at least one wash buffer may comprise of about 100 mM sodium acetate and about 0.1% (w/w) polysorbate 80 with a pH of about 6.0. In certain embodiments, the salt can be NaCl, KCl, MgCl₂, CaCl₂), LiCl, CsCl, sodium acetate (C₂H₃NaO₂), (NH₄)₂SO₄, NH₄Cl, Na₂SO₄, K₂SO₄, sodium citrate, potassium citrate, or a combination thereof. One or more of these wash steps, or even all of these wash steps, are conducted at room temperature. In certain embodiments, the salt is NaCl. In certain embodiments, the wash steps occur at room temperature.

In some embodiments, at least three wash steps are performed; wherein at least one wash buffer comprises from about 50 to about 2000 mM sodium acetate and from about 0.05 to about 0.2% (w/w) polysorbate 80, and a pH from about 5.2 to about 6.8; at least one wash buffer comprises from about 30 to about 200 mM TrisHCl and from about 75 to about 500 mM salt, and a pH from about 7.5 to about 9.2; and at least one wash buffer comprises from about 30 to about 200 mM TrisHCl and from about 30 to about 75% (w/w) ethylene glycol, and a pH from about 7.3 to about 8.8. In certain embodiments, the salt can be NaCl, KCl, MgCl₂, CaCl₂), LiCl, CsCl, sodium acetate (C₂H₃NaO₂), (NH₄)₂SO₄, NH₄Cl, Na₂SO₄, K₂SO₄, sodium citrate, potassium citrate, or a combination thereof. In certain embodiments, the salt is NaCl. One or more of these wash steps, or even all of these wash steps, are conducted at room temperature. In certain embodiments, the wash steps occur at room temperature.

In some embodiments, at least three wash steps are performed. In some embodiments, at least one buffer comprises about 100 mM sodium acetate, about 0.1% (w/w) polysorbate 80, and a pH of about 6.0. In some embodiments, at least one buffer comprises about 50 mM TrisHCl and about 125 mM NaCl, and a pH of about 8.5. In some embodiments, at least one buffer comprises about 50 mM TrisHCl and about 50% (w/w) ethylene glycol, and a pH of about 8.5. One or more of these wash steps, or even all of these wash steps, are conducted at room temperature. In certain embodiments, the wash steps occur at room temperature.

In some embodiments, at least three wash steps are performed; wherein a first wash step comprises applying to the affinity resin a first buffer comprising from about 50 to about 2000 mM sodium acetate and from about 0.05 to about 0.2% (w/w) polysorbate 80, and where the first buffer has a pH from about 5.2 to about 6.8; a second wash step comprises applying to the affinity resin a second buffer comprising from about 30 to about 200 mM TrisHCl and from about 75 to about 500 mM salt, and where the second buffer has a pH from about 7.5 to about 9.2; and a third wash step comprises applying to the affinity resin a third buffer comprising from about 30 to about 200 mM TrisHCl and from about 30 to about 75% (w/w) ethylene glycol, and where the third buffer has a pH from about 7.3 to about 8.8. In certain embodiments, the salt can be NaCl, KCl, MgCl₂, CaCl₂), LiCl, CsCl, sodium acetate (C₂H₃NaO₂), (NH₄)₂SO₄, NH₄Cl, Na₂SO₄, K₂SO₄, sodium citrate, potassium citrate, or a combination thereof. In certain embodiments, the salt is NaCl. One or more of these wash steps, or even all of these wash steps, are conducted at room temperature. In certain embodiments, the wash steps occur at room temperature.

In some embodiments, the method further comprises a fourth wash step that takes place before the first wash step and comprises applying to the affinity resin a fourth buffer comprising from about 10 to about 30 mM TrisHCl and from about 75 to about 250 mM NaCl, and where the fourth buffer has a pH from about 6.5 to about 8.0.

In some embodiments, at least three wash steps are performed; wherein the first buffer comprises about 100 mM sodium acetate, about 0.1% (w/w) polysorbate 80, and where the first buffer has a pH of about 6.0. In some embodiments, the second buffer comprises about 50 mM TrisHCl and about 125 mM NaCl, and where the second buffer has a pH of about 8.5. In some embodiments, the third buffer comprises about 50 mM TrisHCl and about 50% (w/w) ethylene glycol, and where the third buffer has a pH of about 8.5. One or more of these wash steps, or even all of these wash steps, are conducted at room temperature. In certain embodiments, the wash steps occur at room temperature.

In some embodiments, at least three wash steps are performed; a first wash step comprises applying to the affinity resin a first buffer comprising from about 50 to about 200 mM sodium acetate and from about 0.05 to about 0.2% (w/w) polysorbate 80, and where the first buffer has a pH from about 5.5 to about 6.5; a second wash step comprises applying to the affinity resin a second buffer comprising from about 10 to about 70 mM TrisHCl and from about 75 to about 250 mM NaCl, and where the second buffer has a pH from about 8.0 to about 9.0; and a third wash step comprises applying to the affinity resin a third buffer comprising from about 10 to about 70 mM TrisHCl and from about 30 to about 75% (w/w) ethylene glycol, and where the third buffer has a pH from about 8.0 to about 9.0. In some embodiments, the method further comprises a fourth wash step that takes place before the first wash step and comprises applying to the affinity resin a fourth buffer comprising from about 10 to about 30 mM TrisHCl and from about 75 to about 250 mM NaCl, and where the fourth buffer has a pH from about 6.5 to about 8.0. One or more of these wash steps, or even all of these wash steps, are conducted at room temperature. In certain embodiments, the wash steps occur at room temperature.

In some embodiments, at least three wash steps are performed; a first wash step comprises applying to the affinity resin a first buffer comprising from about 50 to about 200 mM sodium acetate and from about 0.05 to about 0.2% (w/w) polysorbate 80, and where the first buffer has a pH from about 5.5 to about 6.5; a second wash step comprises applying to the affinity resin a second buffer comprising from about 10 to about 70 mM TrisHCl and from about 75 to about 250 mM NaCl, and where the second buffer has a pH from about 8.0 to about 9.0; and a third wash step comprises applying to the affinity resin a third buffer comprising from about 10 to about 70 mM TrisHCl and from about 30 to about 75% (w/w) ethylene glycol, and where the third buffer has a pH from about 8.0 to about 9.0. In some embodiments, the method further comprises a fourth wash step that takes place before the first wash step and comprises applying to the affinity resin a fourth buffer comprising from about 10 to about 30 mM TrisHCl and from about 75 to about 250 mM NaCl, and where the fourth buffer has a pH from about 6.5 to about 8.0. One or more of these wash steps, or even all of these wash steps, are conducted at room temperature. In certain embodiments, the wash steps occur at room temperature.

In some embodiments, the first buffer comprises about 100 mM sodium acetate and about 0.1% (w/w) polysorbate 80, and where the first buffer has a pH of about 6.0. In some embodiments, the second buffer comprises about 50 mM TrisHCl and about 125 mM NaCl, and where the second buffer has a pH of about 8.5. In some embodiments, the third buffer comprises about 50 mM TrisHCl and about 50% (w/w) ethylene glycol, and where the third buffer has a pH of about 8.0. One or more of these wash steps, or even all of these wash steps, are conducted at room temperature. In certain embodiments, the wash steps occur at room temperature.

In yet more embodiments, at least three wash steps are performed; a first wash step comprises applying to the affinity resin a first buffer comprising from about 50 to about 500 mM sodium salt of 2-(N-morpholino)ethanesulfonic acid (MES-Na), from about 3 to about 30 mM EDTA, and a solvent/detergent mixture comprising polysorbate 80, DMSO and tri(n-butyl)phosphate (TNBP), and where the first buffer has a pH from about 5.2 to about 6.8; a second wash step comprises applying to the affinity resin a second buffer comprising from about 30 to about 200 mM TrisHCl or Arginine-HCl and from about 75 to about 500 mM salt, and where the second buffer has a pH from about 7.5 to about 9.2; and a third wash step comprises applying to the affinity resin a third buffer comprising from about 20 to about 80 mM Arginine-HCl and from about 50 to about 60% (w/w) sucrose, and where the third buffer has a pH from about 7.3 to about 8.8. In some embodiments, the salt is selected from NaCl, KCl, MgCl₂, CaCl₂, sodium citrate, LiCl, CsCl, sodium acetate, and a combination of one or more of NaCl, KCl, MgCl₂, CaCl₂, sodium citrate, LiCl, CsCl, and sodium acetate. In some embodiments, the salt is NaCl. In some embodiments, the concentration of the salt does not exceed 500 mM. In some embodiments, the concentration of the salt does not exceed 200 mM. In some embodiments, the concentration of salt in the third buffer does not exceed 500 mM. In some embodiments, the concentration of salt in the third buffer does not exceed 200 mM. In some embodiments, the method further comprises a fourth wash step that takes place before the first wash step and comprises applying to the affinity resin a fourth buffer comprising from about 20 to about 100 mM Arginine-HCl and from about 75 to about 250 mM NaCl, and where the fourth buffer has a pH from about 7.5 to about 8.8. One or more of these wash steps, or even all of these wash steps, are conducted at room temperature. In certain embodiments, the wash steps occur at room temperature.

In some embodiments, the method further comprises a wash step that takes place after the first elution step and before a second elution step, the wash step comprising applying to the affinity resin a fifth buffer comprising from about 20 to about 100 mM Arginine-HCl and from about 75 to about 250 mM NaCl, and where the fifth buffer has a pH from about 7.5 to about 8.5. In some embodiments, the second elution step comprises applying to the affinity resin a second elution buffer comprising from about 20 to about 100 mM Arginine-HCl, from about 40 to about 60% (w/w) glycerol, and from about 500 to 1000 mM salt (e.g., NaCl), and where the second elution buffer has a pH from about 7.5 to about 8.5.

The additional wash step may be effective to minimize fronting effects between the first and second elution steps, e.g., providing for elution triggered only by the first and second elution buffers themselves and not from fronting that may result from a mixture of the first and second elution buffers. In some embodiments, the method further comprises a sixth wash step that takes place after the fifth wash step and the second elution step, the wash step comprising applying to the affinity resin a sixth buffer comprising from about 20 to about 100 mM Arginine-HCl and from about 75 to about 250 mM NaCl, and where the fifth buffer has a pH from about 7.5 to about 8.5.

In some embodiments, at least three wash steps are performed; a first wash step comprises applying to the affinity resin a first buffer comprising from about 80 to about 400 mM Bis-Tris, and about 10 to about 20 grams of a solvent/detergent mixture comprising Triton-X100, polysorbate 80 and TNBP in a ratio of about 11:3:3 (by weight), and where the first buffer has a pH from about 5.2 to about 6.8; a second wash step comprises applying to the affinity resin a second buffer comprising from about 5 to about 20 mmol sodium citrate, and where the second buffer has a pH from about 7.5 to about 9.2; and a third wash step comprises applying to the affinity resin a third buffer comprising from about 50 to about 200 mM Arginine-HCl, from about 50 to about 200 mM Lysine HCl, from about 50 to about 200 mM Histidine-HCl, from about 1 mM to about 4 mM N-acetyl-D,L-tryptophan, and about 10% to about 40% (w/w) polysorbate 80, and where the third buffer has a pH from about 7.3 to about 8.8.

In certain embodiments, the first elution step uses a first buffer, which can be a TrisHCl based buffer. In certain embodiments, the first elution step uses a first buffer, which can be a sodium acetate (NaAcetate) based buffer. In certain embodiments, the first elution step uses a first buffer comprising a sodium salt of 2-(N-morpholino)ethanesulfonic acid (MES-Na), EDTA, and a solvent/detergent mixture comprising polysorbate 80, DMSO and tri(n-butyl)phosphate (TNBP). In certain embodiments, the first elution step uses a first buffer comprising from about 50 to about 200 mM taurine, and 0.2 to 1.5% PEG (e.g., PEG 6000). In certain embodiments, the first elution step uses a first buffer comprising Bis-Tris, and a solvent/detergent mixture comprising Triton-X100, polysorbate 80 and TNBP. In certain embodiments, the first elution step uses a first buffer comprising sodium acetate and polysorbate 80. One or more of these elution steps, or even all of these elution steps, are conducted at room temperature. In certain embodiments, the first elution step uses a first buffer, which can be a TrisHCl based buffer composing NaCl.

In certain embodiments, the second elution step uses a second buffer, which can be a TrisHCl based buffer. In certain embodiments, the second elution step uses a second buffer, which can be a sodium acetate (NaAcetate) based buffer. In certain embodiments, the second elution step uses a second buffer comprising a sodium salt of 2-(N-morpholino)ethanesulfonic acid (MES-Na), EDTA, and a solvent/detergent mixture comprising polysorbate 80, DMSO and tri(n-butyl)phosphate (TNBP). In certain embodiments, the second elution step uses a second buffer comprising from about 50 to about 200 mM taurine, and 0.2 to 1.5% PEG (e.g., PEG 6000). In certain embodiments, the second elution step uses a second buffer comprising Bis-Tris, and a solvent/detergent mixture comprising Triton-X100, polysorbate 80 and TNBP. In certain embodiments, the second elution step uses a second buffer comprising sodium acetate and polysorbate 80. In certain embodiments, the second elution step uses a second buffer comprising TrisHCl and NaCl buffer.

In certain embodiments, at least one elution buffer may comprise from about 10 to about 200 mM TrisHCl and from about 50 to about 500 mM salt with a pH of about 7.5 to about 9.2. In certain embodiments, at least one elution buffer may comprise from about 25 to about 100 mM TrisHCl and from about 75 to about 250 mM salt with a pH of about 8.0 to about 9.0. In certain embodiments, at least one elution buffer may comprise from about 40 to about 60 mM TrisHCl and from about 100 to about 150 mM salt with a pH of about 8.0 to about 9.0. In certain embodiments, at least one elution buffer may comprise or about 50 mM TrisHCl and about 125 mM salt with a pH of about 8.5. In certain embodiments, the salt can be NaCl, KCl, MgCl₂, CaCl₂, LiCl, CsCl, sodium acetate (C₂H₃NaO₂), (NH₄)₂SO₄, NH₄Cl, Na₂SO₄, K₂SO₄, sodium citrate, potassium citrate, or a combination thereof. In certain embodiments, the salt is NaCl. In certain embodiments, the elution step(s) occur below 18° C. (e.g., between 1° C. and 12° C. or between 2° C. and 8° C. as discussed above).

In certain embodiments, at least one elution buffer may comprise from about 10 to about 2000 mM sodium acetate and about 0.001 to about 1% (w/w) polysorbate 80 with a pH of about 5.0 to about 7.4. In certain embodiments, at least one elution buffer may comprise from about 50 to about 200 mM sodium acetate and about 0.005 to about 0.3% (w/w) polysorbate 80 with a pH of about 5.5 to about 7.0. In certain embodiments, at least one elution buffer may comprise from about 90 to about 110 mM sodium acetate and about 0.05 to about 0.2% (w/w) polysorbate 80 with a pH of about 5.5 to about 6.5. In certain embodiments, at least one elution buffer may comprise of about 100 mM sodium acetate and about 0.1% (w/w) polysorbate 80 with a pH of about 6.0. In certain embodiments, the elution step(s) occur below 18° C. (e.g., between 1° C. and 12° C. or between 2° C. and 8° C. as discussed above).

In certain embodiments, at least one elution buffer may comprise from about 10 to about 200 mM glycine, about 1 to about 100 mM histidine, about 20 to about 500 mM salt, about 1 to about 10% (w/w) trehalose and about 0.0005 to about 1% (w/w) polysorbate 80 with a pH of about 6.0 to about 8.0. In certain embodiments, at least one elution buffer may comprise from about 30 mM to about 80 mM glycine, about 5 to about 20 mM histidine, about 50 to about 200 mM salt, about 3 to about 8% (w/w) trehalose and about 0.001 to about 0.1% (w/w) polysorbate 80 with a pH of about 6.5 to about 7.5. In certain embodiments, at least one elution buffer may comprise from about 40 to about 60 mM glycine, about 5 to about 15 mM histidine, about 90 to about 110 mM salt, about 4 to about 6% (w/w) trehalose and about 0.001 to about 0.05% (w/w) polysorbate 80 with a pH of about 7.0 to about 7.4. In certain embodiments, at least one elution buffer may comprise about 50 mM glycine, about 10 mM histidine, about 100 mM salt, about 5% (w/w) trehalose, about 0.005% (w/w) polysorbate 80 with a pH of about 7.0. In certain embodiments, the salt can be NaCl, KCl, MgCl₂, CaCl₂, LiCl, CsCl, sodium acetate (C₂H₃NaO₂), (NH₄)₂SO₄, NH₄Cl, Na₂SO₄, K₂SO₄, sodium citrate, potassium citrate, or a combination thereof. In certain embodiments, the salt is NaCl. In certain embodiments, the elution step(s) occur below 18° C. (e.g., between 1° C. and 12° C. or between 2° C. and 8° C. as discussed above).

In certain embodiments, at least one elution buffer may comprise from about 1 to about 200 mM TrisHCl, from about 50 to about 500 mM salt, and from about 0.001 to about 1% (w/w) polysorbate 80 with a pH of about 6.0 to about 8.8. In certain embodiments, at least one elution buffer may comprise about 5 to about 50 mM TrisHCl, from about 75 to about 250 mM salt, and from about 0.005 to about 0.3% (w/w) polysorbate 80 with a pH of about 6.5 to about 8.5. In certain embodiments, at least one elution buffer may comprise about 10 to about 30 mM TrisHCl, from about 140 to about 160 mM salt, and from about 0.05 to about 0.2% (w/w) polysorbate 80 with a pH of about 7.0 to about 8.0. In certain embodiments, at least one elution buffer may comprise about 20 mM TrisHCl about 150 mM salt, and about 0.1% (w/w) polysorbate 80 with a pH of about 7.4. In certain embodiments, the salt can be NaCl, KCl, MgCl₂, CaCl₂, LiCl, CsCl, sodium acetate (C₂H₃NaO₂), (NH₄)₂SO₄, NH₄Cl, Na₂SO₄, K₂SO₄, sodium citrate, potassium citrate, or a combination thereof. In certain embodiments, the salt is NaCl. In certain embodiments, the elution step(s) occur below 18° C. (e.g., between 1° C. and 12° C. or between 2° C. and 8° C. as discussed above).

In certain embodiments, at least one elution buffer may comprise from about 10 to about 200 mM TrisHCl and from about 10 to about 75% (w/w) ethylene glycol with a pH of about 7.5 to about 9.2. In certain embodiments, at least one elution buffer may comprise from about 25 mM to about 100 mM TrisHCl and from about 25% to about 70% (w/w) ethylene glycol with a pH of about 8.0 to about 9.0. In certain embodiments, at least one elution buffer may comprise from about 40 mM to about 60 mM TrisHCl and from about 40% to about 60% (w/w) ethylene glycol with a pH or about 8.0 to about 8.8. In certain embodiments, at least one elution buffer may comprise about 50 mM TrisHCl and about 50% (w/w) ethylene glycol with a pH of about 8.5.

In certain embodiments, at least two elution buffers are used. In certain embodiments, at least one elution buffer may comprise from about 10 to about 200 mM TrisHCl and from about 50 to about 500 mM salt with a pH of about 7.5 to about 9.2. In certain embodiments, at least one elution buffer may comprise from about 10 to about 2000 mM sodium acetate and about 0.001 to about 1% (w/w) polysorbate 80 with a pH of about 5.0 to about 7.4. In certain embodiments, the salt can be NaCl, KCl, MgCl₂, CaCl₂, LiCl, CsCl, sodium acetate (C₂H₃NaO₂), (NH₄)₂SO₄, NH₄Cl, Na₂SO₄, K₂SO₄, sodium citrate, potassium citrate, or a combination thereof. In certain embodiments, the elution step(s) occur below 18° C. (e.g., between 1° C. and 12° C. or between 2° C. and 8° C. as discussed above).

In certain embodiments, at least two elution buffers are used. In certain embodiments, at least one elution buffer may comprise from about 25 to about 100 mM TrisHCl and from about 75 to about 250 mM salt with a pH of about 8.0 to about 9.0. In certain embodiments, at least one elution buffer may comprise from about 50 to about 200 mM sodium acetate and about 0.005 to about 0.3% (w/w) polysorbate 80 with a pH of about 5.5 to about 7.0. In certain embodiments, the salt can be NaCl, KCl, MgCl₂, CaCl₂, LiCl, CsCl, sodium acetate (C₂H₃NaO₂), (NH₄)₂SO₄, NH₄Cl, Na₂SO₄, K₂SO₄, sodium citrate, potassium citrate, or a combination thereof. In certain embodiments, the elution step(s) occur below 18° C. (e.g., between 1° C. and 12° C. or between 2° C. and 8° C. as discussed above).

In certain embodiments, at least two elution buffers are used. In certain embodiments, at least one elution buffer may comprise from about 40 to about 60 mM TrisHCl and from about 100 to about 150 mM salt with a pH of about 8.0 to about 9.0. In certain embodiments, at least one elution buffer may comprise from about 90 to about 110 mM sodium acetate and about 0.05 to about 0.2% (w/w) polysorbate 80 with a pH of about 5.5 to about 6.5. In certain embodiments, the salt can be NaCl, KCl, MgCl₂, CaCl₂, LiCl, CsCl, sodium acetate (C₂H₃NaO₂), (NH₄)₂SO₄, NH₄Cl, Na₂SO₄, K₂SO₄, sodium citrate, potassium citrate, or a combination thereof. In certain embodiments, the elution step(s) occur below 18° C. (e.g., between 1° C. and 12° C. or between 2° C. and 8° C. as discussed above).

In certain embodiments, at least two elution buffers are used. In certain embodiments, at least one elution buffer may comprise or about 50 mM TrisHCl and about 125 mM salt with a pH of about 8.5. In certain embodiments, at least one elution buffer may comprise of about 100 mM sodium acetate and about 0.1% (w/w) polysorbate 80 with a pH of about 6.0. In certain embodiments, the salt can be NaCl, KCl, MgCl₂, CaCl₂, LiCl, CsCl, sodium acetate (C₂H₃NaO₂), (NH₄)₂SO₄, NH₄Cl, Na₂SO₄, K₂SO₄, sodium citrate, potassium citrate, or a combination thereof. In certain embodiments, the elution step(s) occur below 18° C. (e.g., between 1° C. and 12° C. or between 2° C. and 8° C. as discussed above).

In some embodiments, at least three elution steps are performed; wherein at least one elution buffer comprises from about 50 to about 2000 mM sodium acetate and from about 0.05 to about 0.2% (w/w) polysorbate 80, and a pH from about 5.2 to about 6.8; at least one elution buffer comprises from about 30 to about 200 mM TrisHCl and from about 75 to about 500 mM salt, and a pH from about 7.5 to about 9.2; and at least one elution buffer comprises from about 30 to about 200 mM TrisHCl and from about 30 to about 75% (w/w) ethylene glycol, and a pH from about 7.3 to about 8.8. In certain embodiments, the salt can be NaCl, KCl, MgCl₂, CaCl₂, LiCl, CsCl, sodium acetate (C₂H₃NaO₂), (NH₄)₂SO₄, NH₄Cl, Na₂SO₄, K₂SO₄, sodium citrate, potassium citrate, or a combination thereof. In certain embodiments, the salt is NaCl. In certain embodiments, the elution step(s) occur below 18° C. (e.g., between 1° C. and 12° C. or between 2° C. and 8° C. as discussed above).

In some embodiments, at least three elution steps are performed. In some embodiments, at least one buffer comprises about 100 mM sodium acetate, about 0.1% (w/w) polysorbate 80, and a pH of about 6.0. In some embodiments, at least one buffer comprises about 50 mM TrisHCl and about 125 mM NaCl, and a pH of about 8.5. In some embodiments, at least one buffer comprises about 50 mM TrisHCl and about 50% (w/w) ethylene glycol, and a pH of about 8.5. In certain embodiments, the elution step(s) occur below 18° C. (e.g., between 1° C. and 12° C. or between 2° C. and 8° C. as discussed above).

In some embodiments, at least three elution steps are performed; wherein a first elution step comprises applying to the affinity resin a first buffer comprising from about 50 to about 2000 mM sodium acetate and from about 0.05 to about 0.2% (w/w) polysorbate 80, and where the first buffer has a pH from about 5.2 to about 6.8; a second elution step comprises applying to the affinity resin a second buffer comprising from about 30 to about 200 mM TrisHCl and from about 75 to about 500 mM salt, and where the second buffer has a pH from about 7.5 to about 9.2; and a third elution step comprises applying to the affinity resin a third buffer comprising from about 30 to about 200 mM TrisHCl and from about 30 to about 75% (w/w) ethylene glycol, and where the third buffer has a pH from about 7.3 to about 8.8. In certain embodiments, the salt can be NaCl, KCl, MgCl₂, CaCl₂, LiCl, CsCl, sodium acetate (C₂H₃NaO₂), (NH₄)₂SO₄, NH₄Cl, Na₂SO₄, K₂SO₄, sodium citrate, potassium citrate, or a combination thereof. In certain embodiments, the salt is NaCl. In certain embodiments, the elution step(s) occur below 18° C. (e.g., between 1° C. and 12° C. or between 2° C. and 8° C. as discussed above).

In some embodiments, at least one of the elution buffers is at a pH from about 5.8 to about 6.2 and comprises from about 90 to about 110 mM sodium acetate and about 0.09 to about 0.11% (w/w) polysorbate 80/Tween 80. In some embodiments, at least one of the elution buffers is at a pH from about 6.5 to about 7.5 and comprises from about 35 to 70 mM glycine, 5 to 15 mM histidine, 50 to 200 mM NaCl, 3 to 8% trehalose, and 0.001 to 0.005% Crillet™ 4. In some embodiments, at least one of the elution buffers is at a pH from about 8.2 to about 8.8 and comprises from about 45 to about 55 mM TrisHCl and about 90 to about 150 mM NaCl. In certain embodiments, the elution step(s) occur below 18° C. (e.g., between 1° C. and 12° C. or between 2° C. and 8° C. as discussed above).

In some embodiments, the first elution buffer is at a pH from about 5.8 to about 6.2 and comprises from about 90 to about 110 mM sodium acetate and about 0.09 to about 0.11% (w/w) polysorbate 80/Tween 80. In some embodiments, the second elution buffer is at a pH from about 6.5 to about 7.5 and comprises from about 35 to 70 mM glycine, 5 to 15 mM histidine, 50 to 200 mM NaCl, 3 to 8% trehalose, and 0.001 to 0.005% Crillet™ 4. In some embodiments, the third elution buffer is at a pH from about 8.2 to about 8.8 and comprises from about 45 to about 55 mM TrisHCl and about 90 to about 150 mM NaCl. In certain embodiments, the elution step(s) occur below 18° C. (e.g., between 1° C. and 12° C. or between 2° C. and 8° C. as discussed above).

In some embodiments, the first elution buffer is at a pH from about 5.8 to about 6.2 and comprises from about 90 to about 110 mM sodium acetate and about 0.09 to about 0.11% (w/w) polysorbate 80/Tween 80, the second buffer is at a pH from about 8.2 to about 8.8 and comprises from about 45 to about 55 mM TrisHCl and about 110 to about 135 mM NaCl, and the third buffer is at a pH from about 8.2 to about 8.8 and comprises from about 45 to about 55 mM TrisHCl and about 45 to about 55% ethylene glycol. In certain embodiments, there is an optional fourth buffer comprising at a pH from about 7.2 to about 7.6 and comprises about 15 to about 25 mM TrisHCl and about 135 to about 165 mM NaCl. In certain embodiments, the elution buffer is at a pH from about 7.8 to about 8.2 and comprises from about 45 to about 55 mM TrisHCl, about 45 to about 55% ethylene glycol and about 650 to about 850 mM NaCl. In certain embodiments, the elution step(s) occur below 18° C. (e.g., between 1° C. and 12° C. or between 2° C. and 8° C. as discussed above).

In some embodiments, at least one of the elution buffers is at a pH from about 7.2 to about 7.6 and comprises about 15 to about 25 mM TrisHCl and about 135 to about 165 mM NaCl. In some embodiments, at least one of the elution buffers is at a pH from about 5.8 to about 6.2 and comprises from about 90 to about 110 mM sodium acetate and about 0.09 to about 0.11% (w/w) polysorbate 80. In some embodiments, at least one of the elution buffers is at a pH from about 8.2 to about 8.8 and comprises from about 45 to about 55 mM TrisHCl and about 110 to about 135 mM NaCl. In some embodiments, at least one of the elution buffers is at a pH from about 7.5 to about 8.5 and comprises from about 45 to about 55 mM TrisHCl and about 45 to about 55% ethylene glycol. In some embodiments, at least one of the elution buffers is at a pH from about 7.8 to about 8.2 and comprises from about 45 to about 55 mM TrisHCl, about 45 to about 55% ethylene glycol and about 650 to about 850 mM NaCl. In certain embodiments, the elution step(s) occur below 18° C. (e.g., between 1° C. and 12° C. or between 2° C. and 8° C. as discussed above).

In some embodiments, the first buffer is at a pH from about 7.2 to about 7.6 and comprises about 15 to about 25 mM TrisHCl and about 135 to about 165 mM NaCl, the second buffer is at a pH from about 5.8 to about 6.2 and comprises from about 90 to about 110 mM sodium acetate and about 0.09 to about 0.11% (w/w) polysorbate 80, the third buffer is at a pH from about 8.2 to about 8.8 and comprises from about 45 to about 55 mM TrisHCl and about 110 to about 135 mM NaCl, the fourth buffer is at a pH from about 7.5 to about 8.5 and comprises from about 45 to about 55 mM TrisHCl and about 45 to about 55% ethylene glycol, and the fifth elution buffer is at a pH from about 7.8 to about 8.2 and comprises from about 45 to about 55 mM TrisHCl, about 45 to about 55% ethylene glycol and about 650 to about 850 mM NaCl. In certain embodiments, the elution step(s) occur below 18° C. (e.g., between 1° C. and 12° C. or between 2° C. and 8° C. as discussed above).

In certain embodiments, the first wash buffer may comprise from about 10 to about 200 mM TrisHCl and from about 50 to about 500 mM salt with a pH of about 7.5 to about 9.2. In certain embodiments, the first elution buffer may comprise from about 10 to about 200 mM TrisHCl and from about 50 to about 500 mM salt with a pH of about 7.5 to about 9.2. In certain embodiments, the salt can be NaCl, KCl, MgCl₂, CaCl₂, LiCl, CsCl, sodium acetate (C₂H₃NaO₂), (NH₄)₂SO₄, NH₄Cl, Na₂SO₄, K₂SO₄, sodium citrate, potassium citrate, or a combination thereof. In certain embodiments, the salt is NaCl. In certain embodiments, the wash steps occur at room temperature. In certain embodiments, the elution step(s) occur below 18° C. (e.g., between 1° C. and 12° C. or between 2° C. and 8° C. as discussed above).

In certain embodiments, the first wash buffer may comprise from about 25 to about 100 mM TrisHCl and from about 75 to about 250 mM salt with a pH of about 8.0 to about 9.0. In certain embodiments, the first elution buffer may comprise from about 25 to about 100 mM TrisHCl and from about 75 to about 250 mM salt with a pH of about 8.0 to about 9.0. In certain embodiments, the salt can be NaCl, KCl, MgCl₂, CaCl₂, LiCl, CsCl, sodium acetate (C₂H₃NaO₂), (NH₄)₂SO₄, NH₄Cl, Na₂SO₄, K₂SO₄, sodium citrate, potassium citrate, or a combination thereof. In certain embodiments, the salt is NaCl. In certain embodiments, the wash steps occur at room temperature. In certain embodiments, the elution step(s) occur below 18° C. (e.g., between 1° C. and 12° C. or between 2° C. and 8° C. as discussed above).

In certain embodiments, the first wash buffer may comprise from about 40 to about 60 mM TrisHCl and from about 100 to about 150 mM salt with a pH of about 8.0 to about 9.0. In certain embodiments, the first elution buffer may comprise from about 25 to about 100 mM TrisHCl and from about 75 to about 250 mM salt with a pH of about 8.0 to about 8.8. In certain embodiments, the salt can be NaCl, KCl, MgCl₂, CaCl₂, LiCl, CsCl, sodium acetate (C₂H₃NaO₂), (NH₄)₂SO₄, NH₄Cl, Na₂SO₄, K₂SO₄, sodium citrate, potassium citrate, or a combination thereof. In certain embodiments, the salt is NaCl. In certain embodiments, the wash steps occur at room temperature. In certain embodiments, the elution step(s) occur below 18° C. (e.g., between 1° C. and 12° C. or between 2° C. and 8° C. as discussed above).

In certain embodiments, the first wash buffer may comprise or about 50 mM TrisHCl and about 125 mM salt with a pH of about 8.5. In certain embodiments, the first elution buffer may comprise of about 50 mM TrisHCl and about 125 mM salt with a pH of about 8. In certain embodiments, the salt can be NaCl, KCl, MgCl₂, CaCl₂, LiCl, CsCl, sodium acetate (C₂H₃NaO₂), (NH₄)₂SO₄, NH₄Cl, Na₂SO₄, K₂SO₄, sodium citrate, potassium citrate, or a combination thereof. In certain embodiments, the salt is NaCl. In certain embodiments, the wash steps occur at room temperature. In certain embodiments, the elution step(s) occur below 18° C. (e.g., between 1° C. and 12° C. or between 2° C. and 8° C. as discussed above).

In certain embodiments, the first and third wash buffers may comprise from about 10 to about 200 mM TrisHCl and from about 50 to about 500 mM salt with a pH of about 7.5 to about 9.2. In certain embodiments, the first elution buffer may comprise from about 10 to about 200 mM TrisHCl and from about 50 to about 500 mM salt with a pH of about 7.5 to about 9.2. In certain embodiments, the salt can be NaCl, KCl, MgCl₂, CaCl₂, LiCl, CsCl, sodium acetate (C₂H₃NaO₂), (NH₄)₂SO₄, NH₄Cl, Na₂SO₄, K₂SO₄, sodium citrate, potassium citrate, or a combination thereof. In certain embodiments, the salt is NaCl. In certain embodiments, the wash steps occur at room temperature. In certain embodiments, the elution step(s) occur below 18° C. (e.g., between 1° C. and 12° C. or between 2° C. and 8° C. as discussed above).

In certain embodiments, the first and third wash buffers may comprise from about 25 to about 100 mM TrisHCl and from about 75 to about 250 mM salt with a pH of about 8.0 to about 9.0. In certain embodiments, the first elution buffer may comprise from about 25 to about 100 mM TrisHCl and from about 75 to about 250 mM salt with a pH of about 8.0 to about 9.0. In certain embodiments, the salt can be NaCl, KCl, MgCl₂, CaCl₂, LiCl, CsCl, sodium acetate (C₂H₃NaO₂), (NH₄)₂SO₄, NH₄Cl, Na₂SO₄, K₂SO₄, sodium citrate, potassium citrate, or a combination thereof. In certain embodiments, the salt is NaCl. In certain embodiments, the wash steps occur at room temperature. In certain embodiments, the elution step(s) occur below 18° C. (e.g., between 1° C. and 12° C. or between 2° C. and 8° C. as discussed above).

In certain embodiments, the first and third wash buffers may comprise from about 40 to about 60 mM TrisHCl and from about 100 to about 150 mM salt with a pH of about 8.0 to about 9.0. In certain embodiments, the first elution buffer may comprise from about 25 to about 100 mM TrisHCl and from about 75 to about 250 mM salt with a pH of about 8.0 to about 8.8. In certain embodiments, the salt can be NaCl, KCl, MgCl₂, CaCl₂, LiCl, CsCl, sodium acetate (C₂H₃NaO₂), (NH₄)₂SO₄, NH₄Cl, Na₂SO₄, K₂SO₄, sodium citrate, potassium citrate, or a combination thereof. In certain embodiments, the salt is NaCl. In certain embodiments, the wash steps occur at room temperature. In certain embodiments, the elution step(s) occur below 18° C. (e.g., between 1° C. and 12° C. or between 2° C. and 8° C. as discussed above).

In certain embodiments, the first and third wash buffers may comprise or about 50 mM TrisHCl and about 125 mM salt with a pH of about 8.5. In certain embodiments, the first elution buffer may comprise of about 50 mM TrisHCl and about 125 mM salt with a pH of about 8. In certain embodiments, the salt can be NaCl, KCl, MgCl₂, CaCl₂, LiCl, CsCl, sodium acetate (C₂H₃NaO₂), (NH₄)₂SO₄, NH₄Cl, Na₂SO₄, K₂SO₄, sodium citrate, potassium citrate, or a combination thereof. In certain embodiments, the salt is NaCl. In certain embodiments, the wash steps occur at room temperature. In certain embodiments, the elution step(s) occur below 18° C. (e.g., between 1° C. and 12° C. or between 2° C. and 8° C. as discussed above).

In certain embodiments, the first and third wash buffers may comprise from about 10 to about 200 mM TrisHCl and from about 50 to about 500 mM salt with a pH of about 7.5 to about 9.2. In certain embodiments, the second wash buffer may comprise from about 10 to about 2000 mM sodium acetate and about 0.001 to about 1% (w/w) polysorbate 80 with a pH of about 5.0 to about 7.4. In certain embodiments, the first elution buffer may comprise from about 10 to about 200 mM TrisHCl and from about 50 to about 500 mM salt with a pH of about 7.5 to about 9.2. In certain embodiments, the salt can be NaCl, KCl, MgCl₂, CaCl₂, LiCl, CsCl, sodium acetate (C₂H₃NaO₂), (NH₄)₂SO₄, NH₄Cl, Na₂SO₄, K₂SO₄, sodium citrate, potassium citrate, or a combination thereof. In certain embodiments, the salt is NaCl. In certain embodiments, the wash steps occur at room temperature. In certain embodiments, the elution step(s) occur below 18° C. (e.g., between 1° C. and 12° C. or between 2° C. and 8° C. as discussed above).

In certain embodiments, the first and third wash buffers may comprise from about 25 to about 100 mM TrisHCl and from about 75 to about 250 mM salt with a pH of about 8.0 to about 9.0. In certain embodiments, the second wash buffer may comprise from about 50 to about 200 mM sodium acetate and about 0.005 to about 0.3% (w/w) polysorbate 80 with a pH of about 5.5 to about 7.0. In certain embodiments, the first elution buffer may comprise from about 25 to about 100 mM TrisHCl and from about 75 to about 250 mM salt with a pH of about 8.0 to about 9.0. In certain embodiments, the salt can be NaCl, KCl, MgCl₂, CaCl₂, LiCl, CsCl, sodium acetate (C₂H₃NaO₂), (NH₄)₂SO₄, NH₄Cl, Na₂SO₄, K₂SO₄, sodium citrate, potassium citrate, or a combination thereof. In certain embodiments, the salt is NaCl. In certain embodiments, the wash steps occur at room temperature. In certain embodiments, the elution step(s) occur below 18° C. (e.g., between 1° C. and 12° C. or between 2° C. and 8° C. as discussed above).

In certain embodiments, the first and third wash buffers may comprise from about 40 to about 60 mM TrisHCl and from about 100 to about 150 mM salt with a pH of about 8.0 to about 9.0. In certain embodiments, the second wash buffer may comprise from about 90 to about 110 mM sodium acetate and about 0.05 to about 0.2% (w/w) polysorbate 80 with a pH of about 5.5 to about 6.5. In certain embodiments, the first elution buffer may comprise from about 25 to about 100 mM TrisHCl and from about 75 to about 250 mM salt with a pH of about 8.0 to about 8.8. In certain embodiments, the salt can be NaCl, KCl, MgCl₂, CaCl₂, LiCl, CsCl, sodium acetate (C₂H₃NaO₂), (NH₄)₂SO₄, NH₄Cl, Na₂SO₄, K₂SO₄, sodium citrate, potassium citrate, or a combination thereof. In certain embodiments, the salt is NaCl. In certain embodiments, the wash steps occur at room temperature. In certain embodiments, the elution step(s) occur below 18° C. (e.g., between 1° C. and 12° C. or between 2° C. and 8° C. as discussed above).

In certain embodiments, the first and third wash buffers may comprise or about 50 mM TrisHCl and about 125 mM salt with a pH of about 8.5. In certain embodiments, the second wash buffer may comprise about 100 mM sodium acetate and about 0.1% (w/w) polysorbate 80 with a pH of about 6.0. In certain embodiments, the first elution buffer may comprise of about 50 mM TrisHCl and about 125 mM salt with a pH of about 8. In certain embodiments, the salt can be NaCl, KCl, MgCl₂, CaCl₂, LiCl, CsCl, sodium acetate (C₂H₃NaO₂), (NH₄)₂SO₄, NH₄Cl, Na₂SO₄, K₂SO₄, sodium citrate, potassium citrate, or a combination thereof. In certain embodiments, the salt is NaCl. In certain embodiments, the wash steps occur at room temperature. In certain embodiments, the elution step(s) occur below 18° C. (e.g., between 1° C. and 12° C. or between 2° C. and 8° C. as discussed above).

In certain embodiments, the first and third wash buffers may comprise from about 10 to about 200 mM TrisHCl and from about 50 to about 500 mM salt with a pH of about 7.5 to about 9.2. In certain embodiments, the second and fourth wash buffer may comprise from about 10 to about 2000 mM sodium acetate and about 0.001 to about 1% (w/w) polysorbate 80 with a pH of about 5.0 to about 7.4. In certain embodiments, the first elution buffer may comprise from about 10 to about 2000 mM sodium acetate and about 0.001 to about 1% (w/w) polysorbate 80 with a pH of about 5.0 to about 7.4. In certain embodiments, the salt can be NaCl, KCl, MgCl₂, CaCl₂, LiCl, CsCl, sodium acetate (C₂H₃NaO₂), (NH₄)₂SO₄, NH₄Cl, Na₂SO₄, K₂SO₄, sodium citrate, potassium citrate, or a combination thereof. In certain embodiments, the salt is NaCl. In certain embodiments, the wash steps occur at room temperature. In certain embodiments, the elution step(s) occur below 18° C. (e.g., between 1° C. and 12° C. or between 2° C. and 8° C. as discussed above).

In certain embodiments, the first and third wash buffers may comprise from about 25 to about 100 mM TrisHCl and from about 75 to about 250 mM salt with a pH of about 8.0 to about 9.0. In certain embodiments, the second and fourth wash buffer may comprise from about 50 to about 200 mM sodium acetate and about 0.005 to about 0.3% (w/w) polysorbate 80 with a pH of about 5.5 to about 7.0. In certain embodiments, the first elution buffer may comprise from about 50 to about 200 mM sodium acetate and about 0.005 to about 0.3% (w/w) polysorbate 80 with a pH of about 5.5 to about 7.0. In certain embodiments, the salt can be NaCl, KCl, MgCl₂, CaCl₂, LiCl, CsCl, sodium acetate (C₂H₃NaO₂), (NH₄)₂SO₄, NH₄Cl, Na₂SO₄, K₂SO₄, sodium citrate, potassium citrate, or a combination thereof. In certain embodiments, the salt is NaCl. In certain embodiments, the wash steps occur at room temperature. In certain embodiments, the elution step(s) occur below 18° C. (e.g., between 1° C. and 12° C. or between 2° C. and 8° C. as discussed above).

In certain embodiments, the first and third wash buffers may comprise from about 40 to about 60 mM TrisHCl and from about 100 to about 150 mM salt with a pH of about 8.0 to about 9.0. In certain embodiments, the second and fourth wash buffer may comprise from about 90 to about 110 mM sodium acetate and about 0.05 to about 0.2% (w/w) polysorbate 80 with a pH of about 5.5 to about 6.5. In certain embodiments, the first elution buffer may comprise from about 90 to about 110 mM sodium acetate and about 0.05 to about 0.2% (w/w) polysorbate 80 with a pH of about 5.5 to about 6.5. In certain embodiments, the salt can be NaCl, KCl, MgCl₂, CaCl₂, LiCl, CsCl, sodium acetate (C₂H₃NaO₂), (NH₄)₂SO₄, NH₄Cl, Na₂SO₄, K₂SO₄, sodium citrate, potassium citrate, or a combination thereof. In certain embodiments, the salt is NaCl. In certain embodiments, the wash steps occur at room temperature. In certain embodiments, the elution step(s) occur below 18° C. (e.g., between 1° C. and 12° C. or between 2° C. and 8° C. as discussed above).

In certain embodiments, the first and third wash buffers may comprise or about 50 mM TrisHCl and about 125 mM salt with a pH of about 8.5. In certain embodiments, the second and fourth wash buffer may comprise about 100 mM sodium acetate and about 0.1% (w/w) polysorbate 80 with a pH of about 6.0. In certain embodiments, the first elution buffer may comprise of about 100 mM sodium acetate and about 0.1% (w/w) polysorbate 80 with a pH of about 6.0. In certain embodiments, the salt can be NaCl, KCl, MgCl₂, CaCl₂, LiCl, CsCl, sodium acetate (C₂H₃NaO₂), (NH₄)₂SO₄, NH₄Cl, Na₂SO₄, K₂SO₄, sodium citrate, potassium citrate, or a combination thereof. In certain embodiments, the salt is NaCl. In certain embodiments, the wash steps occur at room temperature. In certain embodiments, the elution step(s) occur below 18° C. (e.g., between 1° C. and 12° C. or between 2° C. and 8° C. as discussed above).

In certain embodiments, the first wash buffer may comprise from about 10 to about 2000 mM sodium acetate and about 0.001 to about 1% (w/w) polysorbate 80 with a pH of about 5.0 to about 7.4. In certain embodiments, the first elution buffer may comprise from about 10 to about 2000 mM sodium acetate and about 0.001 to about 1% (w/w) polysorbate 80 with a pH of about 5.0 to about 7.4. In certain embodiments, the elution step(s) occur below 18° C. (e.g., between 1° C. and 12° C. or between 2° C. and 8° C. as discussed above).

In certain embodiments, the first wash buffer may comprise from about 50 to about 200 mM sodium acetate and about 0.005 to about 0.3% (w/w) polysorbate 80 with a pH of about 5.5 to about 7.0. In certain embodiments, the first elution buffer may comprise from about 50 to about 200 mM sodium acetate and about 0.005 to about 0.3% (w/w) polysorbate 80 with a pH of about 5.5 to about 7.0. In certain embodiments, the wash steps occur at room temperature. In certain embodiments, the elution step(s) occur below 18° C. (e.g., between 1° C. and 12° C. or between 2° C. and 8° C. as discussed above).

In certain embodiments, the first wash buffer may comprise from about 90 to about 110 mM sodium acetate and about 0.05 to about 0.2% (w/w) polysorbate 80 with a pH of about 5.5 to about 6.5. In certain embodiments, the first elution buffer may comprise from about 90 to about 110 mM sodium acetate and about 0.05 to about 0.2% (w/w) polysorbate 80 with a pH of about 5.5 to about 6.5. In certain embodiments, the wash steps occur at room temperature. In certain embodiments, the elution step(s) occur below 18° C. (e.g., between 1° C. and 12° C. or between 2° C. and 8° C. as discussed above).

In certain embodiments, the first wash buffer may comprise of about 100 mM sodium acetate and about 0.1% (w/w) polysorbate 80 with a pH of about 6.0. In certain embodiments, the first elution buffer may comprise of about 100 mM sodium acetate and about 0.1% (w/w) polysorbate 80 with a pH of about 6.0. In certain embodiments, the wash steps occur at room temperature. In certain embodiments, the elution step(s) occur below 18° C. (e.g., between 1° C. and 12° C. or between 2° C. and 8° C. as discussed above).

In certain embodiments, the first and third wash buffers may comprise from about 10 to about 2000 mM sodium acetate and about 0.001 to about 1% (w/w) polysorbate 80 with a pH of about 5.0 to about 7.4. In certain embodiments, the first elution buffer may comprise from about 10 to about 2000 mM sodium acetate and about 0.001 to about 1% (w/w) polysorbate 80 with a pH of about 5.0 to about 7.4. In certain embodiments, the wash steps occur at room temperature. In certain embodiments, the elution step(s) occur below 18° C. (e.g., between 1° C. and 12° C. or between 2° C. and 8° C. as discussed above).

In certain embodiments, the first and third wash buffers may comprise from about 50 to about 200 mM sodium acetate and about 0.005 to about 0.3% (w/w) polysorbate 80 with a pH of about 5.5 to about 7.0. In certain embodiments, the first elution buffer may comprise from about 50 to about 200 mM sodium acetate and about 0.005 to about 0.3% (w/w) polysorbate 80 with a pH of about 5.5 to about 7.0. In certain embodiments, the wash steps occur at room temperature. In certain embodiments, the elution step(s) occur below 18° C. (e.g., between 1° C. and 12° C. or between 2° C. and 8° C. as discussed above).

In certain embodiments, the first and third wash buffers may comprise from about 90 to about 110 mM sodium acetate and about 0.05 to about 0.2% (w/w) polysorbate 80 with a pH of about 5.5 to about 6.5. In certain embodiments, the first elution buffer may comprise from about 90 to about 110 mM sodium acetate and about 0.05 to about 0.2% (w/w) polysorbate 80 with a pH of about 5.5 to about 6.5. In certain embodiments, the wash steps occur at room temperature. In certain embodiments, the elution step(s) occur below 18° C. (e.g., between 1° C. and 12° C. or between 2° C. and 8° C. as discussed above).

In certain embodiments, the first and third wash buffers may comprise of about 100 mM sodium acetate and about 0.1% (w/w) polysorbate 80 with a pH of about 6.0. In certain embodiments, the first elution buffer may comprise of about 100 mM sodium acetate and about 0.1% (w/w) polysorbate 80 with a pH of about 6.0. In certain embodiments, the wash steps occur at room temperature. In certain embodiments, the elution step(s) occur below 18° C. (e.g., between 1° C. and 12° C. or between 2° C. and 8° C. as discussed above).

In certain embodiments, the first and third wash buffers may comprise from about 10 to about 2000 mM sodium acetate and about 0.001 to about 1% (w/w) polysorbate 80 with a pH of about 5.0 to about 7.4. In certain embodiments, the second wash buffer may comprise from about 10 to about 200 mM TrisHCl and from about 50 to about 500 mM salt with a pH of about 7.5 to about 9.2. In certain embodiments, the first elution buffer may comprise from about 10 to about 2000 mM sodium acetate and about 0.001 to about 1% (w/w) polysorbate 80 with a pH of about 5.0 to about 7.4. In certain embodiments, the salt can be NaCl, KCl, MgCl₂, CaCl₂, LiCl, CsCl, sodium acetate (C₂H₃NaO₂), (NH₄)₂SO₄, NH₄Cl, Na₂SO₄, K₂SO₄, sodium citrate, potassium citrate, or a combination thereof. In certain embodiments, the salt is NaCl. In certain embodiments, the wash steps occur at room temperature. In certain embodiments, the elution step(s) occur below 18° C. (e.g., between 1° C. and 12° C. or between 2° C. and 8° C. as discussed above).

In certain embodiments, the first and third wash buffers may comprise from about 50 to about 200 mM sodium acetate and about 0.005 to about 0.3% (w/w) polysorbate 80 with a pH of about 5.5 to about 7.0. In certain embodiments, the second wash buffer may comprise from about 25 to about 100 mM TrisHCl and from about 75 to about 250 mM salt with a pH of about 8.0 to about 9.0. In certain embodiments, the first elution buffer may comprise from about 50 to about 200 mM sodium acetate and about 0.005 to about 0.3% (w/w) polysorbate 80 with a pH of about 5.5 to about 7.0. In certain embodiments, the salt can be NaCl, KCl, MgCl₂, CaCl₂, LiCl, CsCl, sodium acetate (C₂H₃NaO₂), (NH₄)₂SO₄, NH₄Cl, Na₂SO₄, K₂SO₄, sodium citrate, potassium citrate, or a combination thereof. In certain embodiments, the salt is NaCl. In certain embodiments, the wash steps occur at room temperature. In certain embodiments, the elution step(s) occur below 18° C. (e.g., between 1° C. and 12° C. or between 2° C. and 8° C. as discussed above).

In certain embodiments, the first and third wash buffers may comprise from about 90 to about 110 mM sodium acetate and about 0.05 to about 0.2% (w/w) polysorbate 80 with a pH of about 5.5 to about 6.5. In certain embodiments, the second wash buffer may comprise from about 40 to about 60 mM TrisHCl and from about 100 to about 150 mM salt with a pH of about 8.0 to about 9.0. In certain embodiments, the first elution buffer may comprise from about 90 to about 110 mM sodium acetate and about 0.05 to about 0.2% (w/w) polysorbate 80 with a pH of about 5.5 to about 6.5. In certain embodiments, the salt can be NaCl, KCl, MgCl₂, CaCl₂, LiCl, CsCl, sodium acetate (C₂H₃NaO₂), (NH₄)₂SO₄, NH₄Cl, Na₂SO₄, K₂SO₄, sodium citrate, potassium citrate, or a combination thereof. In certain embodiments, the salt is NaCl. In certain embodiments, the wash steps occur at room temperature. In certain embodiments, the elution step(s) occur below 18° C. (e.g., between 1° C. and 12° C. or between 2° C. and 8° C. as discussed above).

In certain embodiments, the first and third wash buffers may comprise of about 100 mM sodium acetate and about 0.1% (w/w) polysorbate 80 with a pH of about 6.0. In certain embodiments, the second wash buffer may comprise or about 50 mM TrisHCl and about 125 mM salt with a pH of about 8.5. In certain embodiments, the first elution buffer may comprise of about 100 mM sodium acetate and about 0.1% (w/w) polysorbate 80 with a pH of about 6.0. In certain embodiments, the salt can be NaCl, KCl, MgCl₂, CaCl₂, LiCl, CsCl, sodium acetate (C₂H₃NaO₂), (NH₄)₂SO₄, NH₄Cl, Na₂SO₄, K₂SO₄, sodium citrate, potassium citrate, or a combination thereof. In certain embodiments, the salt is NaCl. In certain embodiments, the wash steps occur at room temperature. In certain embodiments, the elution step(s) occur below 18° C. (e.g., between 1° C. and 12° C. or between 2° C. and 8° C. as discussed above).

In certain embodiments, the first and third wash buffers may comprise from about 10 to about 2000 mM sodium acetate and about 0.001 to about 1% (w/w) polysorbate 80 with a pH of about 5.0 to about 7.4. In certain embodiments, the second and fourth wash buffer may comprise from about 10 to about 200 mM TrisHCl and from about 50 to about 500 mM salt with a pH of about 7.5 to about 9.2. In certain embodiments, the first elution buffer may comprise from about 10 to about 200 mM TrisHCl and from about 50 to about 500 mM salt with a pH of about 7.5 to about 9.2. In certain embodiments, the salt can be NaCl, KCl, MgCl₂, CaCl₂, LiCl, CsCl, sodium acetate (C₂H₃NaO₂), (NH₄)₂SO₄, NH₄Cl, Na₂SO₄, K₂SO₄, sodium citrate, potassium citrate, or a combination thereof. In certain embodiments, the salt is NaCl. In certain embodiments, the wash steps occur at room temperature. In certain embodiments, the elution step(s) occur below 18° C. (e.g., between 1° C. and 12° C. or between 2° C. and 8° C. as discussed above).

In certain embodiments, the first and third wash buffers may comprise from about 50 to about 200 mM sodium acetate and about 0.005 to about 0.3% (w/w) polysorbate 80 with a pH of about 5.5 to about 7.0. In certain embodiments, the second and fourth wash buffer may comprise from about 25 to about 100 mM TrisHCl and from about 75 to about 250 mM salt with a pH of about 8.0 to about 9.0. In certain embodiments, the first elution buffer may comprise from about 25 to about 100 mM TrisHCl and from about 75 to about 250 mM salt with a pH of about 8.0 to about 9.0. In certain embodiments, the salt can be NaCl, KCl, MgCl₂, CaCl₂, LiCl, CsCl, sodium acetate (C₂H₃NaO₂), (NH₄)₂SO₄, NH₄Cl, Na₂SO₄, K₂SO₄, sodium citrate, potassium citrate, or a combination thereof. In certain embodiments, the salt is NaCl. In certain embodiments, the wash steps occur at room temperature. In certain embodiments, the elution step(s) occur below 18° C. (e.g., between 1° C. and 12° C. or between 2° C. and 8° C. as discussed above).

In certain embodiments, the first and third wash buffers may comprise from about 90 to about 110 mM sodium acetate and about 0.05 to about 0.2% (w/w) polysorbate 80 with a pH of about 5.5 to about 6.5. In certain embodiments, the second and fourth wash buffer may comprise from about 40 to about 60 mM TrisHCl and from about 100 to about 150 mM salt with a pH of about 8.0 to about 9.0. In certain embodiments, the first elution buffer may comprise from about 40 to about 60 mM TrisHCl and from about 100 to about 150 mM salt with a pH of about 8.0 to about 9.0. In certain embodiments, the salt can be NaCl, KCl, MgCl₂, CaCl₂, LiCl, CsCl, sodium acetate (C₂H₃NaO₂), (NH₄)₂SO₄, NH₄Cl, Na₂SO₄, K₂SO₄, sodium citrate, potassium citrate, or a combination thereof. In certain embodiments, the salt is NaCl. In certain embodiments, the wash steps occur at room temperature. In certain embodiments, the elution step(s) occur below 18° C. (e.g., between 1° C. and 12° C. or between 2° C. and 8° C. as discussed above).

In certain embodiments, the first and third wash buffers may comprise of about 100 mM sodium acetate and about 0.1% (w/w) polysorbate 80 with a pH of about 6.0. In certain embodiments, the second and fourth wash buffer may comprise or about 50 mM TrisHCl and about 125 mM salt with a pH of about 8.5. In certain embodiments, the first elution buffer may comprise of about 50 mM TrisHCl and about 125 mM salt with a pH of about 8.5. In certain embodiments, the salt can be NaCl, KCl, MgCl₂, CaCl₂, LiCl, CsCl, sodium acetate (C₂H₃NaO₂), (NH₄)₂SO₄, NH₄Cl, Na₂SO₄, K₂SO₄, sodium citrate, potassium citrate, or a combination thereof. In certain embodiments, the salt is NaCl. In certain embodiments, the wash steps occur at room temperature. In certain embodiments, the elution step(s) occur below 18° C. (e.g., between 1° C. and 12° C. or between 2° C. and 8° C. as discussed above).

Additional examples of wash and/or elution buffers can be found in WO2019133677, which is incorporated herein in its entirety for all intended purposes.

### Resin

In some embodiments, the affinity resin is POROS™ CaptureSelect™ AAVx.

In some embodiments, the AAV is AAV8, the affinity resin is POROS™ CaptureSelect™ AAV8. In some embodiments, the AAV is AAV9, the affinity resin is POROS™ CaptureSelect™ AAV9. In some embodiments, the AAV is AAV9, the affinity resin is POROS™ CaptureSelect™ AAVx.

In some embodiments, the AAV is AAV8, the affinity resin is POROS™ CaptureSelect™ AAV8, and the elution buffer is acidic and does not comprise ethylene glycol. In some embodiments, the AAV is AAV9, the affinity resin is POROS™ CaptureSelect™ AAV9, and where the elution buffer is acidic and does not comprise ethylene glycol. In some embodiments, the AAV is AAV9, the affinity resin is POROS™ CaptureSelect™ AAVx, and where the elution buffer is acidic and does not comprise ethylene glycol.

In some embodiments, the AAV is AAV8, and where the affinity resin is an immune affinity resin consisting of an immobilized monoclonal antibody against AAV8 from type ADK8 or ADK8/9 immobilized on a chromatography matrix. In some embodiments, the AAV is AAV9, and where the affinity resin is an immune affinity resin consisting of an immobilized monoclonal antibody against AAV9 from type ADK9 or ADK8/9 immobilized on a chromatography matrix.

Source of rAAV Particles

With regard to the methods of the present disclosure, the AAV may be of any AAV serotype. In certain embodiments, the AAV purified by the methods described herein are of AAV1 serotype, AAV2 serotype, AAV3 serotype, AAV4 serotype, AAV5 serotype, AAV6 serotype, AAV7 serotype, AAV8 serotype, AAV9 serotype, AAV10 serotype, AAV11 serotype, AAV12 serotype, AAV13 serotype, AAAV serotype, BAAV serotype, AAV (VR-195) serotype, and AAV (VR-355) serotype, or chimeric AAV vectors. In certain embodiments, the AAV is wild type.

In certain embodiments, the AAV is of the AAV5 serotype. In certain embodiments, the AAV is of the AAV9 serotype. In certain embodiments, the AAV is modified by genetic engineering and/or is chemically modified. In certain embodiments, the AAV comprises a modified capsid, e.g., a genetically engineered or a chemically-modified AAV capsid. In certain embodiments, the AAV vector comprise a VP1 comprising the sequence of SEQ ID NO: 1. In certain embodiments, the AAV vector comprise a VP2 comprising the sequence of SEQ ID NO: 2. In certain embodiments, the AAV vector comprise a VP3 comprising the sequence of SEQ ID NO: 3.

In some embodiments, the AAV is modified by genetic engineering and/or is chemically modified. In certain embodiments, the AAV is genetically and/or chemically modified. In certain embodiments, the AAV is genetically modified. In certain embodiments, the AAV is chemically modified.

In certain embodiments, the AAV has been genetically modified to create AAV vectors with altered receptor usage, antigenicity, transduction efficiency and/or tissue tropism for gene therapy application. In certain embodiments, the AAV may be genetically modified to insert peptide ligands, antibodies, antibody fragments, MHCs, and/or receptors into the viral capsid. In certain embodiments, the AAV may be genetically modified to insert peptides for labeling of the viral capsid. Non-limiting examples of possible modifications can be found in Bianing H., Bolyard C. M., Hallek M., Bartlett J. S. (2012) Modification and Labeling of AAV Vector Particles. In: Snyder R., Moullier P. (eds) Adeno-Associated Virus. Methods in Molecular Biology (Methods and Protocols), vol 807. Humana Press, which is incorporated herein in its entirety for all intended purposes.

In certain embodiments, the AAV have been chemically modified to alter the AAV vector's tissue tropism. For example, chemoselective reactions that can target specific amino acid side chains can be exploited to alter the charge, polarity, hydrophobicity and hydrogen bonding potential within receptor binding domains on AAV capsids. Such ability to alter specific receptor make-up on the AAV capsid surface allows for the generation of synthetic vectors with altered tissue tropism. In certain embodiments, chemically modified AAV vectors can exhibit altered receptor usage, antigenicity, transduction efficiency and/or tissue tropism of the chemically modified AAV vectors. Non-limiting examples of possible modifications can be found in Bining H., Bolyard C. M., Hallek M., Bartlett J. S. (2012) Modification and Labeling of AAV Vector Particles. In: Snyder R., Moullier P. (eds) Adeno-Associated Virus. Methods in Molecular Biology (Methods and Protocols), vol 807. Humana Press, which is incorporated herein in its entirety for all intended purposes.

In certain embodiments, the AAV fraction represents an AAV fraction produced by transfected host cells. In certain embodiments, the AAV fraction represents a supernatant harvested from a cell culture comprising host cells transfected with a triple plasmid system, where one plasmid of the system comprises a gene or cDNA of interest, one plasmid encodes capsid protein VP1, capsid protein VP2 and/or capsid protein VP3. In certain embodiments, VP1, VP2, and/or VP3 are AAV8 VP1, AAV8 VP2, and/or AAV8 VP3. In certain embodiments, VP1, VP2, and/or VP3 are AAV9 VP1, AAV9 VP2, and/or AAV9 VP3. Triple plasmid transfection for purposes of rAAV production is known in the art. See, e.g., Qu et al., 2015, supra, and Mizukami et al., “A Protocol for AAV vector production and purification.” PhD dissertation, Division of Genetic Therapeutics, Center for Molecular Medicine, 1998; and Kotin et al., Hum Mol Genet 20(R1): R2-R6 (2011). In certain embodiments, the transfection may be carried out using inorganic compounds, e.g., calcium phosphate, or organic compounds, polyethyleneimine (PEI), or non-chemical means, e.g., electroporation. In certain embodiments, the host cells are adherent cells. In certain embodiments, the host cells are suspension cells. In certain embodiments, the host cells are HEK293 cells or Sf9 cells. In certain embodiments, the cell culture comprises culture medium which is serum and protein free. In certain embodiments, the medium is chemically defined and is free of animal derived components, e.g., hydrolysates. In certain embodiments, the fraction comprising rAAV particles represents a fraction comprising HEK293 cells transfected with a triple plasmid system. In certain embodiments, the fraction comprising rAAV particles is described in U.S. Provisional Application No. 62/417,775 and WO2018128688, which is incorporated herein by reference for all intended purposes.

After culturing host cells, e.g., HEK293 cells, to produce AAV particles (e.g., AAV8, AAV9, etc.), and the clarified cell free culture supernatant is concentrated and/or filtered, the viral particles are loaded onto the affinity matrix. In certain embodiments, the viral particles are loaded in solution having a pH ranging from about 7.4 to about 7.8. In certain embodiments, the viral particles are loaded in solution having a pH ranging from about 8.3 to about 8.7. In certain embodiments, the viral particles are loaded in a solution having a pH of about 8.5. In certain embodiments, the pH is from 8.3 to 8.7 and the solution comprises NaCl and TrisHCl. In certain embodiments, the viral particles are loaded in a solution comprising about 20 mM TrisHCl and about 150 mM NaCl, and having a pH of about 8.5.

### Additional Steps and Considerations

The methods of the present disclosure comprise any combination of steps disclosed herein, and may optionally be combined with one or more additional steps. Accordingly, in exemplary aspects, the methods of the present disclosure further comprise the step of transfecting host cells with a triple plasmid system as described herein. In exemplary aspects, the methods of the present disclosure comprise harvesting a supernatant from a cell culture comprising host cells, e.g., HEK293 cells, transfected with a triple plasmid system. In exemplary aspects, the transfection and harvesting step occurs prior to the ultracentrifugation step described herein.

The methods of the present disclosure may comprise yet other additional steps, which may further increase the purity of the AAV and remove other unwanted components and/or concentrate the fraction and/or condition the fraction for a subsequent step.

In certain embodiments, an optional reequilibration step is performed prior to the wash steps listed above.

In certain embodiments, pre-purification can be undertaken to remove one or more of complex acidic protein structures and host cell DNA, prior to affinity purification of the AAV-containing solution from host cell production. Pre-purification may be conducted by anion exchange in flow through mode. The pre-purification step may be undertaken before any of the affinity purification methods described herein. One of more of the following may be removed by pre-purification of such AAV-containing solution: histones (e.g., histone H2A type 1, histone H2B type 1-B, histone H4, histone H1.4), 60S ribosomal proteins (e.g., 60S ribosomal protein L27, 60S ribosomal protein L6 and 60S ribosomal protein L30), cytoplasmic actin (e.g., cytoplasmic actin 1), tubulin (e.g., tubulin beta-2A chain), heterogeneous nuclear ribonucleoprotein C, Rep68 protein, HEK293 laminin receptor 37 kDa form (LamR 37 kDa) and ATP-dependent molecular chaperone HSC82.

In certain embodiments, the additional step can be ultracentrifugation step.

In exemplary aspects, the method comprises a depth filtration step. In exemplary aspects, the method comprises subjecting a fraction of a transfected HEK293 cell culture supernatant to depth filtration using a filter comprising cellulose and perlites and having a minimum permeability of about 500 L/m². In exemplary aspects, the method further comprises use of a filter having a minimum pore size of about 0.2 μm. In exemplary aspects, the depth filtration is followed by filtration through the filter having a minimum pore size of about 0.2 μm. In exemplary aspects, one or both of the depth filter and filter having a minimum pore size of about 0.2 μm are washed and the washes are collected. In exemplary aspects, the washes are pooled together and combined with the filtrate obtained upon depth filtration and filtration with the filter having a minimum pore size of about 0.2 μm.

In some embodiments, the method further comprises contacting the AAV-containing solution with an anion exchanger and eluting the AAV containing solution from the anion exchanger prior to loading the AAV containing solution onto the affinity resin. The anion exchanger may be operated in flow-through mode.

In exemplary aspects, the methods of the present disclosure comprise one or more chromatography steps. In exemplary aspects, the methods comprise a negative chromatography step whereby unwanted components bind to the chromatography resin and the desired AAV does not bind to the chromatography resin. In exemplary aspects, the methods comprise a negative anion exchange (AEX) chromatography step, or an AEX chromatography step in the “non-binding mode”. Advantages of “non-binding mode” include relative ease of carrying out the procedure and in conducting subsequent assaying.

Accordingly, in exemplary embodiments, the methods of purifying AAV particles comprise performing negative anion exchange (AEX) chromatography on a fraction comprising AAV particles by applying the fraction to an AEX chromatography column or membrane under conditions that allow for the AAV to flow through the AEX chromatography column or membrane and collecting AAV particles. In exemplary aspects, the fraction is applied to the AEX chromatography column or membrane with a loading buffer comprising about 100 mM to about 150 mM salt, e.g., NaCl, optionally, where the pH of the loading buffer is about 8 to about 9. In exemplary aspects, the loading buffer comprises about 115 mM to about 130 mM salt, e.g., NaCl, optionally, where the loading buffer comprises about 120 mM to about 125 mM salt, e.g., NaCl. In exemplary aspects, the negative AEX step occurs prior to the ultracentrifugation step described herein.

In exemplary aspects, the methods of the present disclosure comprise concentrating an AAV fraction using an ultra/diafiltration system. In exemplary aspects, the methods of the present disclosure comprise one more tangential flow filtration (TFF) steps. In exemplary aspects, the AAV fraction undergoes ultra-/dia-filtration. In exemplary aspects, the AAV fraction is concentrated with the ultra/diafiltration system before a step comprising performing negative AEX chromatography, after a step comprising performing negative AEX chromatography, or before and after comprising performing negative AEX chromatography. In exemplary aspects, the TFF steps occur prior to the ultracentrifugation step described herein.

The inactivation of enveloped viruses can be of particular importance, for example when a Baculo transfection system is used. In exemplary aspects, the methods of the present disclosure comprise filtration of a fraction comprising rAAV particles to remove viruses of greater size than the rAAV particles in the fraction.

Without wishing to be bound by theory, ethylene glycol on its own, or in combination with another additive, can inactivate such lipid enveloped viruses. Exemplary additives include nonionic detergents, aliphatic agents (e.g., TnBP), and detergents (e.g., polysorbate (e.g., Tween), Triton X100, TnBP). For example, the solvent-detergent mixture can comprise 1% Triton X100, 0.3% Tri-N-butyl phosphate, and 0.3% TWEEN 80.

The inactivation of lipid enveloped viruses “on column” can be tested in various affinity chromatography runs, as summarized in Table 1 below.

TABLE 1
Solvent detergent treatments used in Variant A, B and C

| Step at which | | | |
|---|---|---|---|
| Detergent | | | |
| Solvent was | | | |
| Applied | Variant A | Variant B | Variant C |
| WASH 2 | Potential SD- | None | Established SD- |
| Possible on | Treatment | | Treatment |
| step: | 10 to 30 g/kg | | 16.6 g S/D solution of |
| LOAD, | of a mixture | | 10.87 g Triton × 100 |
| WASH 1, | of 18.0 g | | 3.31 g Polysorbate 80 |
| WASH 2, | Tween 80 | | 3.01 g TnBP |
| WASH 3, | | | |
| WASH 4, | 3.4 g DMSO, | | |
| ELUATE | 3.6 g TnBP | | |
| WASH 4 | None | Elevated pH 8.5 in | 1 to 20% (w/w) |
| Possible on | | presence of 50 to | Polysorbate 80 |
| step: | | 60%(w/w) Ethylene | Possible in eluate |
| WASH 1, | | glycol and detergent | |
| WASH 2, | | e.g., 0.1-10% | |
| WASH 3, | | Octylglyopyranoside | |
| WASH4 | | Not in eluate | |
| WASH x | None | None | Polar organic solvent |
| | | | 50% |
| | | | Dimethlysufoxide |

The DMSO containing buffer Wash X buffer may be effective to trigger elution of AAV9, but not AAV8, on a CaptureSelect AAV8 resin at near to neutral pH (e.g., pH 8.0), a result which was surprising. The DMSO containing buffer Wash X buffer may be effective to trigger elution of various AAVs, including but not limited to, AAV1, AAV2, AAV3, AAV4, AAV5, AAV6, AAV7, AAV8, AAV9, AAV10, AAV11, AAV12, AAV13, AAAV, BAAV, AAV (VR-195), and AAV (VR-355), on a CaptureSelect AAVx resin at near to neutral pH (e.g., pH 8.0), a result which was surprising. In certain embodiments, the AAV is of the AAV5 serotype. In certain embodiments, the AAV is of the AAV6 serotype. In certain embodiments, the AAV is of the AAV8 serotype. In certain embodiments, the AAV is of the AAV9 serotype.

Without wishing to be bound by theory, the Wash X buffer is expected to have the activities of washing the column and/or inactivating or disintegrating lipid-enveloped viruses. There was no expectation that the Wash X buffer would differentially elute AAV9 over AAV8.

In exemplary aspects, the methods of the present disclosure comprise one or more quality control steps, e.g., steps to measure the potency or specific activity of the AAV fractions obtained after one or more steps (e.g., after each step) of the process. In exemplary aspects, the methods of the present disclosure comprise an ELISA specific for AAV. In exemplary aspects, the ELISA is a sandwich ELISA. In exemplary aspects, the sandwich ELISA comprises an antibody specific for an AAV epitope. In exemplary aspects, the AAV epitope is a conformational epitope present on assembled AAV capsids. As discussed herein, the ELISA may replace qPCR as a way to determine potency of an AAV fraction. In exemplary aspects, the methods of the present disclosure comprise testing an AAV fraction via an AAV-specific ELISA and the methods do not include a method of measuring potency via quantitative PCR. In exemplary aspects, the AAV-specific ELISA is sufficient to provide a representative reading on potency of the AAV fraction, because the majority of the capsids in the AAV fraction are full capsids.

In exemplary aspects, the methods of the present disclosure comprise an ELISA specific for AAV after one or more of the steps of the present disclosure. In exemplary aspects, the methods of the present disclosure comprise testing an AAV fraction obtained after depth filtration via an AAV-specific ELISA to determine the specific activity of the AAV in that fraction. In exemplary aspects, the methods of the present disclosure comprise testing an AAV fraction obtained after concentrating an AAV fraction using an ultra-/diafiltration system via an AAV-specific ELISA to determine the specific activity of the AAV in that fraction. In exemplary aspects, the methods of the present disclosure comprise testing an AAV fraction obtained after a tangential flow filtration (TFF) step via an AAV-specific ELISA to determine the specific activity of the AAV in that fraction. In exemplary aspects, the methods of the present disclosure comprise testing an AAV fraction obtained after negative anion exchange (AEX) chromatography via an AAV-specific ELISA to determine the specific activity of the AAV in that fraction. In exemplary aspects, the methods of the present disclosure comprise testing an AAV fraction obtained after a polish step via an AAV-specific ELISA to determine the specific activity of the AAV in that fraction.

In some embodiments, the method further comprises contacting the AAV containing solution with a filter comprising positively charged groups effective to deplete acidic charged contaminants from the AAV containing solution. In some embodiments, the method further comprises nanofiltration of an AAV fraction to remove viruses greater than 35 nm. In some embodiments, the method further comprises a polish step comprising performing cation exchange chromatography. Exemplary media for use in cation exchange chromatography include, but is not limited to, Capto™ S, Eshmuno® S, and Fractogel® SO3. In some embodiments, the method further comprises testing an AAV fraction via an AAV-specific ELISA, e.g., specific for AAV8 or specific for AAV9. The AAV specific ELISA may be a sandwich ELISA specific for AAV, e.g., AAV8 or AAV9.

### AAV Product

In another aspect is provided an AAV product produced by any method described herein.

An AAV product produced by a method of the present disclosures is further provided herein. In exemplary aspects, the AAV product comprises at least about 10¹² virus particles (vp) produced from about 1000 L of starting material (e.g., cell culture) or at least about 10¹³ virus particles (vp) produced from about 1000 L of starting material (e.g., cell culture). In exemplary aspects, the AAV product is an empty capsid, generated by transfecting the rep-cap and Ad helper plasmids without the transgene plasmid. Purified empty plasmids can be used to deplete or remove antibodies specific to AAV antigens from the blood of a patient.

In exemplary aspects, the AAV product of the present disclosures is highly pure, highly potent and suitable for clinical use in humans. In exemplary aspects, the AAV product comprises AAV particles of a homogenous population and high purity. In exemplary aspects, the AAV product comprises full-length vector DNA. In exemplary embodiments, the AAV product is substantially free of unwanted contaminants, including but not limited to, AAV particles containing truncated or incomplete vector DNA, AAV particles with incomplete protein composition and oligomerized structures, or contaminating viruses, e.g., non AAV, lipid enveloped viruses. In exemplary embodiments, the AAV product contains a high amount of encoding cDNA of the protein of interest. In exemplary aspects, the AAV product of the present disclosure is suitable for administration to a human. In exemplary aspects, the AAV product is sterile and/or of good manufacturing practice (GMP) grade. In exemplary aspects, the AAV product conforms to the requirements set forth in the U.S. Pharmacopeia Chapter 1046 or the European Pharmacopoeia on gene therapy medicinal products or as mandated by the U.S. Food and Drug Administration (USFDA) or the European Medicines Agency (EMA). In exemplary aspects, the AAV product is a ready-to-use product for direct administration to a human with little to no processing or handling.

With regard to the methods of the invention, the AAV fraction is in exemplary aspects a concentrated AAV fraction. In certain embodiments, the AAV fraction comprises at least 1×10¹⁰, 1×10¹¹ or 1×10¹² AAV capsids per mL. In certain embodiments, the AAV fraction comprises at least 1×10¹² AAV capsids per mL. The AAV capsids may include empty AAV capsids and full AAV capsids. In certain embodiments, the AAV fraction comprises more full AAV capsids than empty AAV and/or overfilled AAV capsids.

In certain embodiments, at least about 25%, 30%, 35%, 40%, 45%, 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of the AAV capsids eluted from the elution step is full AAV capsids.

In certain embodiments, the methods of producing and purifying AAV described herein results in a product that comprises at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 50%, 55%, 60%, 65%, 70%, 75%, 80%, 85%, 90%, or 95%, more full capsids than that obtained by a comparative procedure without the same wash protocol and elution at a lower temperature. In certain embodiments, the methods of producing and purifying AAV described herein results in a product that comprises about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 50%, 55%, 60%, 65%, 70%, 75%, 80%, 85%, 90%, or 95%, more full capsids than that obtained by a comparative procedure without the same wash protocol and elution at a lower temperature. In certain embodiments, the methods of producing and purifying AAV described herein results in a product that comprises at least about 6% more full capsids than that obtained by a comparative procedure without the same wash protocol and elution at a lower temperature. In certain embodiments, the methods of producing and purifying AAV described herein results in a product that comprises at least about 10% more full capsids than that obtained by a comparative procedure without the same wash protocol and elution at a lower temperature. In certain embodiments, the methods of producing and purifying AAV described herein results in a product that comprises at least about 20% more full capsids than that obtained by a comparative procedure without the same wash protocol and elution at a lower temperature. In certain embodiments, the methods of producing and purifying AAV described herein results in a product that comprises about 6% more full capsids than that obtained by a comparative procedure without the same wash protocol and elution at a lower temperature. In certain embodiments, the methods of producing and purifying AAV described herein results in a product that comprises about 10% more full capsids than that obtained by a comparative procedure without the same wash protocol and elution at a lower temperature. In certain embodiments, the methods of producing and purifying AAV described herein results in a product that comprises about 16% more full capsids than that obtained by a comparative procedure without the same wash protocol and elution at a lower temperature. In certain embodiments, the methods of producing and purifying AAV described herein results in a product that comprises about 20% more full capsids than that obtained by a comparative procedure without the same wash protocol and elution at a lower temperature.

In various embodiments, the yield of AAV, e.g., AAV9, after the purification steps described herein and as measured by an ITR-qPCR assay as weight/volume, is at least 10%, 15%, 20%, 25%, 30%, 35%, 40%, 45%, 50%, 55%, 60%, or 65% greater than that obtained by a comparative procedure in which no wash steps are performed.

In various embodiments, the yield of AAV, e.g., AAV9, after the purification steps described herein and as measured by an ITR-qPCR assay as weight/weight, is at least 10%, 15%, 20%, 25%, 30%, 35%, 40%, 45%, 50%, 55%, 60%, or 65% greater than that obtained by a comparative procedure in which no wash steps are performed.

Advantageously, the methods are scalable to large volumes of starting material, e.g., cell culture. In certain embodiments, the methods provided herein are large-scale methods capable of purifying AAV from volumes of at least or about 500 L, at least or about 600 L, at least or about 700 L, at least or about 800 L, at least or about 900 L, or at least or about 1000 L. In certain embodiments, the methods are scalable to a minimum volume of starting material (e.g., cell culture) of at least or about 1250 L, at least or about 1500 L, at least or about 2000 L, at least or about 2500 L, at least or about 3000 L, at least or about 4000 L, at least or about 5000 L, at least or about 6000 L, at least or about 7000 L, at least or about 8000 L, at least or about 9000 L, at least or about 10,000 L, or more. For example, the methods are carried out with a minimum volume of about 1000 L or about 10,000 L or 25,000 L or more cell culture producing AAV.

The methods of producing and purifying AAV described herein are also advantageous, because the methods result in high titer AAV production. In certain embodiments, an AAV product comprising at least about 10¹⁰ virus particles (vp) is produced from about 1000 L of starting material (e.g., cell culture). In certain embodiments, an AAV product comprising at least about 10¹¹ virus particles (vp) is produced from about 1000 L of starting material (e.g., cell culture). In certain embodiments, an AAV product comprising at least about 10¹² virus particles (vp) is produced from about 1000 L of starting material (e.g., cell culture). In certain embodiments, an AAV product comprising at least about 10¹³ virus particles (vp) is produced from about 1000 L of starting material (e.g., cell culture). In certain embodiments, an AAV product comprising at least about 10¹⁴ virus particles (vp) is produced from about 1000 L of starting material (e.g., cell culture). In certain embodiments, an AAV product comprising at least about 10¹⁵ virus particles (vp) is produced from about 1000 L of starting material (e.g., cell culture). In certain embodiments, an AAV product comprising at least about 10¹⁶ virus particles (vp) is produced from about 1000 L of starting material (e.g., cell culture). In certain embodiments, an AAV product comprising at least about 10¹⁷ virus particles (vp) is produced from about 1000 L of starting material (e.g., cell culture). In certain embodiments, an AAV product comprising at least about 2×10¹⁶ virus particles (vp) is produced from about 1000 L of starting material (e.g., cell culture). In certain embodiments, an AAV product comprising at least about 5×10¹⁷ virus particles (vp) is produced from about 1000 L of starting material (e.g., cell culture).

Another advantage of the methods described herein is that the methods yield a highly pure AAV product. In certain embodiments, the AAV product produced through the methods of the present disclosure is substantially free of one or more contaminants: host cell proteins, host cell nucleic acids (e.g., free host cell DNA and free plasmid DNA), plasmid DNA, empty viral capsids, heat shock protein 70 (HSP70), lactate dehydrogenase (LDH), proteasomes, contaminant non-AAV viruses (e.g., lipid-enveloped viruses), host cell culture components (e.g., antibiotics), mycoplasma, pyrogens, bacterial endotoxins, cell debris (e.g., debris composed of membrane lipids, proteins and other biological polymers), and adventitious agents. One or more, or even all of, the following impurities may be undetectable when AAV is purified according to the methods of producing and purifying AAV described herein: histone H2A type 1, histone H2B type 1-B, histone H4, heat shock 70 kDa protein 1A, pyruvate kinase PKM, elongation factor 2, ATP-citrate synthase, histone H1.4, immunoglobulin heavy constant gamma 1 (immobilized ligand from an acidic elution method), 60S ribosomal protein L27, fructose-bisphosphate aldolase A, heat shock cognate 71 kDa protein, cytoplasmic actin 1, S-formylglutathione hydrolase, asparagine synthetase (glutamine hydrolyzing), L-lactate dehydrogenase B chain, tubulin beta-2A chain, X-chromosome RNA-binding motif protein, 60S ribosomal protein L6, cytoplasmic threonine tRNA ligase, immunoglobulin kappa constant, 60S ribosomal protein L30, WD repeat-containing protein 1, adenosylhomocysteinase, heterogeneous nuclear ribonucleoprotein C, protein Rep68, thimet oligopeptidase, D-3-phosphoglycerate dehydrogenase, ATP-dependent molecular chaperone HSC82. Adding an anion exchange step prior to the wash steps, according to methods of producing and purifying anion AAV described herein, can also render the following undetectable: histone H1.4, 60S ribosomal protein L27, cytoplasmic actin 1, tubulin beta-2A chain, 60S ribosomal protein L6, 60S ribosomal protein L30, heterogeneous nuclear ribonucleoprotein C, protein Rep68, and ATP-dependent molecular chaperone HSC82.

In exemplary embodiments, the methods of the present disclosure provide a purified AAV product where at least or about 50% of the contaminant found in the starting material (e.g., cell culture) is removed. In exemplary embodiments, the methods of the present disclosure provide a purified AAV product where at least or about 60% of the contaminant found in the starting material (e.g., cell culture) is removed. In exemplary embodiments, the methods of the present disclosure provide a purified AAV product where at least or about 70% of the contaminant found in the starting material (e.g., cell culture) is removed. In exemplary embodiments, the methods of the present disclosure provide a purified AAV product where at least or about 80% of the contaminant found in the starting material (e.g., cell culture) is removed. In exemplary embodiments, the methods of the present disclosure provide a purified AAV product where at least or about 90% of the contaminant found in the starting material (e.g., cell culture) is removed.

In certain embodiments, the methods of producing and purifying AAV described herein reduce the number of impurities, including protein impurities (e.g., host cell (HC) impurities), by about 10%, 15%, 20%, 25%, 30%, 35%, 40%, 45%, 50%, 55%, 60%, 65%, 70%, 75%, 80%, 85%, 90%, 95%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99%, 99.1%, 99.2% of greater than that obtained by a comparative procedure without the same wash protocol. In some embodiments, the AAV obtained from the eluting step has an impurity level of s 99.9%. In some embodiments, the AAV obtained from the eluting step has an impurity level of s 99.0%.

In certain embodiments, the methods of producing and purifying AAV described herein results in a product that is at least about 10%, 15%, 20%, 25%, 30%, 35%, 40%, 45%, 50%, 55%, 60%, 65%, 70%, 75%, 80%, 85%, 90%, 95%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99%, 99.1%, 99.2% pure. In some embodiments, the AAV obtained from the eluting step has a purity level of 99.9% or greater. In some embodiments, the AAV obtained from the eluting step has a purity level of 99.0% or greater.

In certain embodiments, the methods of producing and purifying AAV described herein results in a product that is at least about 10%, 15%, 20%, 25%, 30%, 35%, 40%, 45%, 50%, 55%, 60%, 65%, 70%, 75%, 80%, 85%, 90%, 95%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99%, 99.1%, 99.2% purer than that obtained by a comparative procedure without the same wash protocol and elution at a lower temperature. In some embodiments, the AAV obtained from the eluting step has a purity level of 99.9% or greater than that obtained by a comparative procedure without the same wash protocol and elution at a lower temperature. In some embodiments, the AAV obtained from the eluting step has a purity level of 99.0% or greater than that obtained by a comparative procedure without the same wash protocol and elution at a lower temperature.

In certain embodiments, the AAV product produced through the methods of the present disclosure is suitable for administration to a human. In certain embodiments, the AAV is a recombinant AAV (rAAV). In certain embodiments, the AAV product produced through the methods of the present disclosure is sterile and/or of good manufacturing practice (GMP) grade. In certain embodiments, the AAV product produced through the methods of the present disclosure conforms to the requirements set forth in the U.S. Pharmacopeia Chapter 1046 or the European Pharmacopoeia on gene therapy medicinal products or as mandated by the U.S. Food and Drug Administration (USFDA) or the European Medicines Agency (EMA).

Additionally, the AAV product produced from the methods described herein are highly potent. The potency of an AAV product, e.g., an AAV8 or AAV9 product, can be described in terms of (1) in vivo biopotency (e.g., production of active protein in mice) which is given as units (FIX or FVIII) per mL of mouse plasma; or (2) in vitro biopotency. The in vitro biopotency test measures the potential of AAV vectors to infect cells, e.g., HepG2 cells, which express and secrete the protein of interest into the medium, and determine the amount by ELISA techniques and/or enzyme activity. Suitable methods of measuring in vivo and in vitro biopotency are known in the art and also described herein.

In further embodiments, the AAV product produced from the methods described herein demonstrate superior specific activity. The “Specific activity” of the AAV is represented by the ratio of qPCR to μg AAV8. In exemplary embodiments, the AAV product produced from the methods described herein demonstrate a superior ratio of GOI per μg of AAV demonstrating that the AAV product has a high amount of “full” virus particles. In certain embodiments, the methods of the present disclosure comprise testing an AAV fraction via an AAV-specific ELISA. In certain embodiments, the AAV-specific ELISA is sufficient to provide a representative reading on potency of the AAV fraction, because the majority of capsids in the AAV fraction are full capsids.

### Non-Limiting Embodiments

1. A method of purifying an adeno-associated virus (AAV) comprising
(a) loading an AAV containing solution onto an affinity resin targeted against the AAV at room temperature and under conditions that allow binding between the AAV in the solution and the affinity resin;
(b) undertaking at least one wash step at room temperature; and
(c) eluting the AAV from the affinity resin at a temperature of less than 18° C.
2. The method of embodiment 1, wherein the temperature in step (c) is between 1° C. and 12° C.
3. The method of embodiment 1, wherein the temperature in step (c) is between 2° C. and 8° C.
4. The method of any one of embodiments 1 to 3, further comprising contacting the AAV containing solution with an anion exchanger and eluting the AAV containing solution from the anion exchanger prior to loading the AAV containing solution onto the affinity resin.
5. The method of any one of embodiments 1 to 4, wherein at least one of the wash steps comprises applying to the affinity resin a buffer comprising an organic solvent and/or detergent.
6. The method of embodiment 5, wherein the buffer comprises sodium acetate.
7. The method of embodiment 5, wherein the buffer comprises TrisHCl and a salt.
8. The method of embodiment 5, wherein the buffer comprises one or more of Histidine, Histidine-HCl, Arginine-HCl, Lysine-HCl, Glycine, Taurine, MES-Na, Bis-Tris, and N-acetyl-D,L-tryptophan.
9. The method of embodiment 7 or embodiment 8, wherein the salt is NaCl.
10. The method of embodiment 5, wherein the buffer comprises magnesium chloride.
11. The method of embodiment 5, wherein the buffer comprises TrisHCl and ethylene glycol.
12. The method of embodiment 5, wherein the buffer comprises Arginine-HCl and one of sucrose and glycerol.
13. The method of embodiment 5, wherein the buffer comprises Taurine and ethylene glycol.
14. The method of embodiment 5, wherein the buffer comprises Arginine-HCl, Lysine-HCl, and Histidine-HCl.
15. The method of embodiment 5, wherein the buffer comprises TrisHCl and DMSO.
16. The method of embodiment 5, wherein the organic solvent or detergent is polysorbate 80, ethylene glycol, sorbitol, mannitol, xylitol, DMSO, sucrose, or trehalose.
17. The method of embodiment 5, wherein the detergent comprises one or more of Triton X100, polysorbate 80, and tri (n-butyl) phosphate (TNBP).
18. The method of embodiment 17, wherein the detergent comprises polysorbate 80.
19. The method of any one of embodiments 5 to 18, wherein the buffer comprises from about 0.05% to about 30% (w/w) organic solvent or detergent.
20. The method of embodiment 5, wherein the buffer comprises from about 0.05% to about 0.2% (w/w) organic solvent or detergent.
21. The method of any one of embodiments 1 to 20, wherein at least two wash steps are performed at room temperature.
22. The method of any one of embodiments 1 to 21, wherein at least three wash steps are performed at room temperature.
23. The method of any one of embodiments 1 to 22, wherein at least four wash steps are performed at room temperature.
24. The method of embodiment 21, wherein two wash steps are performed.
25. The method of embodiment 22, wherein three wash steps are performed.
26. The method of embodiment 23, wherein four wash steps are performed.
27. The method of any one of embodiments 1 to 26, wherein the wash steps are performed in succession.
28. The method of any one of embodiments 1 to 27, wherein at least one wash buffer comprises from about 10 to about 200 mM TrisHCl and from about 50 to about 500 mM salt.
29. The method of embodiment 28, wherein at least one wash buffer comprises from about 25 to about 100 mM TrisHCl and from about 75 to about 250 mM salt
30. The method of embodiment 29, wherein at least one wash buffer comprises from about 40 to about 60 mM TrisHCl and from about 100 to about 150 mM salt.
31. The method of any one of embodiments 28 to 30, wherein the wash buffer has a pH from about 7.5 to about 9.2, about 8.0 to about 9.0, or about 8.0 to about 8.8.
32. The method of embodiment 31, wherein at least one wash buffer comprises about 50 mM TrisHCl and about 125 mM salt, and has a pH of about 8.5.
33. The method of any one of embodiments 1 to 32, wherein at least one wash buffer comprises from about 10 to about 2000 mM sodium acetate and from about 0.001 to about 1% (w/w) polysorbate 80.
34. The method of embodiment 33, wherein at least one wash buffer comprises from about 50 to about 200 mM sodium acetate and from about 0.005 to about 0.3% (w/w) polysorbate 80.
35. The method of embodiment 34, wherein at least one wash buffer comprises from about 90 to about 110 mM sodium acetate and from about 0.05 to about 0.2% (w/w) polysorbate 80.
36. The method of any one of embodiments 33 to 35, wherein the wash buffer has a pH from about 5.0 to about 7.4, about 5.5 to about 7.0, or about 5.5 to about 6.5.
37. The method of embodiment 36, wherein at least one wash buffer comprises about 100 mM sodium acetate and about 0.1% (w/w) polysorbate 80, and has a pH of about 6.0.
38. The method of any one of embodiments 1 to 37, wherein at least one wash buffer comprises from about 10 to about 200 mM TrisHCl and from about 10 to about 75% (w/w) ethylene glycol.
39. The method of embodiment 38, wherein at least one wash buffer comprises from about 25 mM to about 100 mM TrisHCl and from about 25% to about 70% (w/w) ethylene glycol.
40. The method of embodiment 39, wherein at least one wash buffer comprises from about 40 mM to about 60 mM TrisHCl and from about 40% to about 60% (w/w) ethylene glycol.
41. The method of any one of embodiments 38 to 40, wherein the wash buffer has a pH from about 7.5 to about 9.2, about 8.0 to about 9.0, or about 8.0 to about 8.8.
42. The method of claim 41, wherein at least one wash buffer comprises about 50 mM TrisHCl and about 50% (w/w) ethylene glycol, and has a pH of about 8.5.
43. The method of any one of embodiments 1 to 42, wherein at least one wash buffer comprises from about 10 to about 200 mM glycine, about 1 to about 100 mM histidine, about 20 to about 500 mM salt, about 1 to about 10% (w/w) trehalose and about 0.0005 to about 1% (w/w) polysorbate 80.
44. The method of embodiment 43, wherein at least one wash buffer comprises from about 30 mM to about 80 mM glycine, about 5 to about 20 mM histidine, about 50 to about 200 mM salt, about 3 to about 8% (w/w) trehalose and about 0.001 to about 0.1% (w/w) polysorbate 80.
45. The method of embodiment 44, wherein at least one wash buffer comprises from about 40 to about 60 mM glycine, about 5 to about 15 mM histidine, about 90 to about 110 mM salt, about 4 to about 6% (w/w) trehalose and about 0.001 to about 0.05% (w/w) polysorbate 80.
46. The method of any one of embodiments 43 to 45, wherein the wash buffer has a pH from about 6.0 to about 8.0, about 6.5 to about 7.5, or about 7.0 to about 7.4.
47. The method of embodiment 46, wherein at least one wash buffer comprises about 50 mM glycine, about 10 mM histidine, about 100 mM salt, about 5% (w/w) trehalose, about 0.005% (w/w) polysorbate 80, and has a pH of about 7.0.
48. The method of any one of embodiments 1 to 47, wherein at least one wash buffer comprises from about 1 to about 200 mM TrisHCl, from about 50 to about 500 mM salt, and from about 0.001 to about 1% (w/w) polysorbate 80.
49. The method of embodiment 48, wherein at least one wash buffer comprises from about 5 to about 50 mM TrisHCl, from about 75 to about 250 mM salt, and from about 0.005 to about 0.3% (w/w) polysorbate 80.
50. The method of embodiment 49, wherein at least one wash buffer comprises from about 10 to about 30 mM TrisHCl, from about 140 to about 160 mM salt, and from about 0.05 to about 0.2% (w/w) polysorbate 80.
51. The method of any one of embodiments 48 to 50, wherein the wash buffer has a pH from about 6.0 to about 8.8, about 6.5 to about 8.5, or about 7.0 to about 8.0.
52. The method of embodiment 51, wherein at least one wash buffer comprises about 20 mM TrisHCl and about 150 mM salt, and about 0.1% (w/w) polysorbate 80, and has a pH of about 7.4.
53. The method of any one of embodiments 1 to 52, wherein step (c) comprises eluting the AAV with at least one elution buffer.
54. The method of embodiment 53, wherein at least one elution buffer is the same as at least one of the wash buffers.
55. The method of embodiment 54, wherein at least one elution buffer is the same as the last wash buffer used in the final wash step before eluting the AAV in step (c).
56. The method of embodiment 54, wherein the first elution buffer is the same as the last wash buffer used in the final wash step before eluting the AAV in step (c).
57. The method of any one of embodiments 53 to 56, wherein at least one elution buffer comprises from about 10 to about 200 mM glycine, about 1 to about 100 mM histidine, about 20 to about 500 mM salt, about 1 to about 10% (w/w) trehalose, and about 0.0005 to about 1% (w/w) polysorbate 80.
58. The method of embodiment 57, wherein at least one elution buffer comprises from about 30 to about 80 mM glycine, about 5 to about 20 mM histidine, about 50 to about 200 mM salt, about 3 to about 8% trehalose, and about 0.001 to about 0.1% (w/w) polysorbate 80.
59. The method of embodiment 58, wherein at least one elution buffer comprises from about 40 to about 60 mM glycine, about 5 to about 15 mM histidine, about 90 to about 110 mM salt, about 4 to about 6% (w/w) trehalose, and about 0.001 to about 0.05% (w/w) polysorbate 80.
60. The method of any one of embodiments 57 to 59, wherein the wash buffer has a pH from about 6.0 to about 8.0, about 6.5 to about 7.5, or about 7.0 to about 7.4.
61. The method of embodiment 60, wherein at least one elution buffer comprises about 50 mM glycine, about 10 mM histidine, about 100 mM salt, about 5% (w/w) trehalose, and about 0.005% (w/w) polysorbate 80, and has a pH of about 7.0.
62. The method of any one of embodiments 53 to 61, wherein at least one wash buffer comprises from about 1 to about 200 mM TrisHCl, from about 50 to about 500 mM salt, and from about 0.001 to about 1% (w/w) polysorbate 80.
63. The method of embodiment 62, wherein at least one wash buffer comprises from about 5 to about 50 mM TrisHCl, from about 75 to about 250 mM salt, and from about 0.005 to about 0.3% (w/w) polysorbate 80.
64. The method of embodiment 63, wherein at least one wash buffer comprises from about 10 to about 30 mM TrisHCl, from about 140 to about 160 mM salt, and from about 0.05% to about 0.2% (w/w) polysorbate 80.
65. The method of any one of embodiments 62 to 64, wherein the wash buffer has a pH from about 6.0 to about 8.8, about 6.5 to about 8.5, or about 7.0 to about 8.0.
66. The method of embodiment 65, wherein at least one wash buffer comprises about 20 mM TrisHCl, about 150 mM salt, and about 0.1% (w/w) polysorbate 80 and has a pH of about 7.4.
67. The method of any one of embodiments 1 to 66, wherein the first and third wash steps comprise applying to the affinity resin a buffer comprising from about 10 to about 200 mM TrisHCl and from about 50 to about 500 mM salt with a pH from about 7.5 to about 9.2, and wherein the second wash step comprises applying to the affinity resin a buffer comprising from about 10 to about 2000 mM sodium acetate and from about 0.001 to about 1% (w/w) polysorbate 80 with a pH from about 5.0 to about 7.4.
68. The method of any one of embodiments 1 to 66, wherein the first and third wash steps comprise applying to the affinity resin a buffer compressing from about 10 to about 2000 mM sodium acetate and from about 0.001 to about 1% (w/w) polysorbate 80 with a pH from about 5.0 to about 7.4, and wherein the second wash step comprises applying to the affinity resin a buffer comprising from about 10 to about 200 mM TrisHCl and from about 50 to about 500 mM salt with a pH from about 7.5 to about 9.2.
69. The method of any one of embodiments 1 to 66, wherein the first and third wash steps comprise applying to the affinity resin a buffer compressing from about 10 to about 200 mM TrisHCl and from about 50 to about 500 mM salt with a pH from about 7.5 to about 9.2, and
wherein the second and fourth wash step comprises applying to the affinity resin a buffer comprising about 10 to about 2000 mM sodium acetate and from about 0.001 to about 1% (w/w) polysorbate 80 with a pH from about 5.0 to about 7.4.
70. The method of any one of embodiments 1 to 66, wherein the first and third wash steps comprise applying to the affinity resin a buffer comprising from about 10 to about 2000 mM sodium acetate and from about 0.001 to about 1% (w/w) polysorbate 80 with a pH from about 5.0 to about 7.4, and wherein the second and fourth wash step comprises applying to the affinity resin a buffer compressing from about 10 to about 200 mM TrisHCl and from about 50 to about 500 mM salt with a pH from about 7.5 to about 9.2.
71. The method of embodiment 67 or embodiment 69, wherein the first and third buffer comprises about 50 mM TrisHCl and about 125 mM salt, and has a pH of about 8.5.
72. The method of embodiment 68 or embodiment 70, wherein the first and third buffer comprises about 100 mM sodium acetate, about 0.1% (w/w) polysorbate 80, and has a pH of about 6.0.
73. The method of embodiment 67 or embodiment 69, wherein the second buffer comprises about 100 mM sodium acetate, about 0.1% (w/w) polysorbate 80, and has a pH of about 6.0.
74. The method of embodiment 68 or embodiment 70, wherein the second buffer comprises about 50 mM TrisHCl and about 125 mM salt, and has a pH of about 8.5.
75. The method of embodiment 69, wherein the fourth buffer comprises about 100 mM sodium acetate, about 0.1% (w/w) polysorbate 80, and has a pH of about 6.0.
76. The method of embodiment 70, wherein the fourth buffer comprises about 50 mM TrisHCl and about 125 mM salt, and has a pH of about 8.5.
77. The method of embodiment 67 or embodiment 70, wherein step (c) comprises applying to the affinity resin a buffer comprising about 10 to about 200 mM TrisHCl and from about 50 to about 500 mM salt, and has a pH from about 7.5 to about 9.2.
78. The method of embodiment 68 or embodiment 69 wherein step (c) comprises applying to the affinity resin a buffer comprising about 10 to about 2000 mM sodium acetate and from about 0.001 to about 1% (w/w) polysorbate 80, and has a pH from about 5.0 to about 7.4.
79. The method of embodiment 77, wherein the buffer comprises about 50 mM TrisHCl and about 125 mM salt, and has a pH of about 8.5.
80. The method of embodiment 78, wherein the buffer comprises about 100 mM sodium acetate, about 0.1% (w/w) polysorbate 80, and has a pH of about 6.0.
81. The method of any one of embodiments 1 to 80, wherein at least one wash and/or elution buffer comprises from about 50 to about 500 mM sodium salt of 2-(N-morpholino)ethanesulfonic acid (MES-Na), from about 3 to about 30 mM EDTA, and a solvent/detergent mixture comprising polysorbate 80, DMSO and tri(n-butyl)phosphate (TNBP), and has a pH from about 5.2 to about 6.8.
82. The method of any one of embodiments 1 to 81, wherein at least one wash and/or elution buffer comprises from about 30 to about 200 mM TrisHCl or Arginine-HCl and from about 75 to about 500 mM salt, and has a pH from about 7.5 to about 9.2.
83. The method of any one of embodiments 1 to 82, wherein at least one wash and/or elution buffer comprises from about 20 to about 80 mM Arginine-HCl and from about 50 to about 200 mM salt, and has a pH from about 7.3 to about 8.8.
84. The method of any one of embodiments 1 83, wherein at least one wash and/or elution buffer comprises from about 50 to about 200 mM taurine, and 0.2 to 1.5% (w/w) PEG (e.g., PEG 6000), and has a pH from about 5.2 to about 6.8.
85. The method of any one of embodiments 1 to 84, wherein at least one and/or elution wash buffer comprises from about 30 to about 300 mM glycine, and has a pH from about 7.5 to about 9.2.
86. The method of any one of embodiments 1 to 85, wherein at least one wash and/or elution buffer comprises from about 20 to about 150 mM taurine, from about 30 to about 75% (w/w) ethylene glycol, and from 0.05 to 0.2% octylglycopyranoside, and has a pH from about 7.3 to about 8.8.
87. The method of any one of embodiments 1 to 86, wherein at least one wash and/or elution buffer comprises from about 80 to about 400 mM Bis-Tris, and about 10 to about 20 grams of a solvent/detergent mixture comprising about Triton-X100, polysorbate 80 and TNBP in a ratio of about 11:3:3 (by weight), and has a pH from about 5.2 to about 6.8.
88. The method of any one of embodiments 1 to 87 wherein at least one wash and/or elution buffer comprises from about 5 to about 20 mmol sodium citrate, and has a pH from about 7.5 to about 9.2.
89. The method of any one of embodiments 1 to 88, wherein at least one wash and/or elution buffer comprises from about 50 to about 200 mM Arginine-HCl, from about 50 to about 200 mM Lysine HCl, from about 50 to about 200 mM Histidine-HCl, and from about 1 mM to about 4 mM N-acetyl-D,L-tryptophan, and about 10% to about 40% (w/w) polysorbate 80, and has a pH from about 7.3 to about 8.8.
90. The method of any one of embodiments 1 to 66, wherein the first wash step comprises applying to the affinity resin a first buffer comprising from about 50 to about 2000 mM sodium acetate and from about 0.05 to about 0.2% (w/w) polysorbate 80, and wherein the first buffer has a pH from about 5.2 to about 6.8; wherein the second wash step comprises applying to the affinity resin a second buffer comprising from about 30 to about 200 mM TrisHCl and from about 75 to about 500 mM salt, and wherein the second buffer has a pH from about 7.5 to about 9.2; and wherein the third wash step comprises applying to the affinity resin a third buffer comprising from about 30 to about 200 mM TrisHCl and from about 30 to about 75% (w/w) ethylene glycol, and wherein the third buffer has a pH from about 7.3 to about 8.8.
91. The method of any one of embodiments 1 to 66, wherein the first wash step comprises applying to the affinity resin a first buffer comprising from about 50 to about 500 mM sodium salt of 2-(N-morpholino)ethanesulfonic acid (MES-Na), from about 3 to about 30 mM EDTA, and a solvent/detergent mixture comprising polysorbate 80, DMSO and tri(n-butyl)phosphate (TNBP), and wherein the first buffer has a pH from about 5.2 to about 6.8;
wherein the second wash step comprises applying to the affinity resin a second buffer comprising from about 30 to about 200 mM TrisHCl or Arginine-HCl and from about 75 to about 500 mM salt, and wherein the second buffer has a pH from about 7.5 to about 9.2; and
wherein the third wash step comprises applying to the affinity resin a third buffer comprising from about 20 to about 80 mM Arginine-HCl and from about 50 to about 200 mM salt, and wherein the third buffer has a pH from about 7.3 to about 8.8.
92. The method of any one of embodiments 1 to 66, wherein the first wash step comprises applying to the affinity resin a first buffer comprising from about 50 to about 200 mM taurine, and 0.2 to 1.5% (w/w) PEG (e.g., PEG 6000) wherein the first buffer has a pH from about 5.2 to about 6.8;
wherein the second wash step comprises applying to the affinity resin a second buffer comprising from about 30 to about 300 mM glycine, and wherein the second buffer has a pH from about 7.5 to about 9.2; and
wherein the third wash step comprises applying to the affinity resin a third buffer comprising from about 20 to about 150 mM taurine, from about 30 to about 75% (w/w) ethylene glycol, and from 0.05 to 0.2% (w/w) octylglycopyranoside, and wherein the third buffer has a pH from about 7.3 to about 8.8.
93. The method of any one of embodiments 1 to 66, wherein the first wash step comprises applying to the affinity resin a first buffer comprising from about 80 to about 400 mM Bis-Tris, and about 10 to about 20 grams of a solvent/detergent mixture comprising about Triton-X100, polysorbate 80 and TNBP in a ratio of about 11:3:3 (by weight) wherein the first buffer has a pH from about 5.2 to about 6.8;
wherein the second wash step comprises applying to the affinity resin a second buffer comprising from about 5 to about 20 mmol sodium citrate, and wherein the second buffer has a pH from about 7.5 to about 9.2; and
wherein the third wash step comprises applying to the affinity resin a third buffer comprising from about 50 to about 200 mM Arginine-HCl, from about 50 to about 200 mM Lysine HCl, from about 50 to about 200 mM Histidine-HCl, and from about 1 mM to about 4 mM N-acetyl-D,L-tryptophan, and about 10% to about 40% (w/w) polysorbate 80, and wherein the third buffer has a pH from about 7.3 to about 8.8.
94. The method of any one of embodiments 1 to 66, wherein the first wash step comprises applying to the affinity resin a first buffer comprising from about 50 nM to about 200 mM NaAcetate and from about 0.05 to about 0.2% (w/w) polysorbate 80, wherein the first buffer has a pH of about 5.2 to about 6.8;
wherein the second wash step comprises applying to the affinity resin a second buffer comprising from about 20 nM to about 100 mM TrisHCl and from about 50 nM to about 200 nM of salt, wherein the second buffer has a pH of about 7.5 to about 8.8; and
wherein the third wash step comprises applying to the affinity resin a third buffer comprising about 20 mM to 100 mM TrisHCl, from about 40% to about 60% (w/w) ethylene glycol, and wherein the third buffer has a pH from about 7.5 to about 8.8.
95. The method of any one of embodiments 1 to 66, wherein the first wash step comprises applying to the affinity resin a first buffer comprising from about 50 nM to about 200 mM NaAcetate and from about 0.05 to about 0.2% (w/w) polysorbate 80, wherein the first buffer has a pH of about 5.2 to about 6.8;
wherein the second wash step comprises applying to the affinity resin a second buffer comprising from about 20 nM to about 100 mM TrisHCl and from about 50 nM to about 200 nM of salt, wherein the second buffer has a pH of about 7.5 to about 8.8; and
wherein the third wash step comprises applying to the affinity resin a third buffer comprising about 20 mM to 100 mM TrisHCl, from about 40% to about 60% (w/w) ethylene glycol, and wherein the third buffer has a pH from about 7.5 to about 8.8.
96. The method of any one of embodiments 7, or 29 to 95, wherein the salt is selected from NaCl, KCl, MgCl₂, CaCl₂), Sodium Citrate, LiCl, CsCl, Sodium Acetate, and a combination of one or more of NaCl, KCl, MgCl₂, CaCl₂), Sodium Citrate, LiCl, CsCl, and Sodium Acetate.
97. The method of embodiment 96, wherein the salt is NaCl.
98. The method of any one of embodiments 7, or 29 to 97, wherein the concentration of the salt does not exceed 500 mM.
99. The method of any one of embodiments 7, or 29 to 97, wherein the concentration of the salt does not exceed 200 mM.
100. The method of any one of embodiments 28 to 32 or 67 to 95, wherein the buffer comprises about 50 mM TrisHCl and about 125 mM NaCl with a pH of about 8.5.
101. The method of any one of embodiments 1 to 66, wherein the first wash step comprises applying to the affinity resin a first buffer comprising from about 50 to about 200 mM sodium acetate and from about 0.05 to about 0.2% (w/w) polysorbate 80, and wherein the first buffer has a pH from about 5.5 to about 6.5;
wherein the second wash step comprises applying to the affinity resin a second buffer comprising from about 10 to about 70 mM TrisHCl and from about 75 to about 250 mM NaCl, and wherein the second buffer has a pH from about 8.0 to about 9.0; and
wherein the third wash step comprises applying to the affinity resin a third buffer comprising from about 10 to about 70 mM TrisHCl and from about 30 to about 75% (w/w) ethylene glycol, and wherein the third buffer has a pH from about 8.0 to about 9.0.
102. The method of embodiment 101, further comprising a fourth wash step that takes place before the first wash step and comprises applying to the affinity resin a fourth buffer comprising from about 10 to about 30 mM TrisHCl and from about 75 to about 250 mM NaCl, and wherein the fourth buffer has a pH from about 6.5 to about 8.0.
103. The method of embodiment 100 or embodiment 102, wherein the first buffer comprises about 100 mM sodium acetate and about 0.1% (w/w) polysorbate 80, and wherein the first buffer has a pH of about 6.0.
104. The method of any one of embodiments 100 to 103, wherein the second buffer comprises about 50 mM TrisHCl and about 125 mM NaCl, and wherein the second buffer has a pH of about 8.5.
105. The method of any one of embodiments 100 to 104, wherein the third buffer comprises about 50 mM TrisHCl and about 50% (w/w) ethylene glycol, and wherein the third buffer has a pH of about 8.0.
106. The method of any one of embodiments 1 to 105, wherein an acidic component is removed.
107. The method of embodiment 106, wherein the acidic component is host cell DNA, such as HEK293 DNA, and wherein the acidic component is reduced to a value below 250 pg per microgram of AAV antigen as measured by qPCR.
108. The method of embodiment 106, wherein the acidic component is host cell DNA, such as HEK293 DNA, and wherein the acidic component is reduced to a value below 250 pg per microgram of AAV antigen as measured by ELISA.
109. The method of any one of embodiments 1 to 108, wherein eluting comprises applying a continuous linear increase of the conductivity of an elution buffer by gradient elution.
110. The method of any one of embodiments 1 to 109, wherein eluting comprises applying a continuous linear increase of the concentration of an organic solvent by gradient elution.
111. The method of any one of embodiments 1 to 110, wherein eluting comprises contacting the affinity resin with an elution buffer comprising sodium acetate, glycine, histidine, NaCl, and/or polysorbate 80.
112. The method of embodiment 111, wherein the salt concentration is about 50 to 200 mM.
113. The method of embodiment 111 or embodiment 112, wherein the pH is from 5.5 to 9.0.
114. The method of any one of embodiments 111 to 113, wherein the eluting comprises contacting the affinity resin with an elution buffer comprising 50 to 200 mM NaCl and 30 to 80 mM TrisHCl.
115. The method of embodiment 114 wherein the elution buffer is at a pH of 8.0 to 9.0.
116. The method of embodiment 114, wherein the elution buffer comprises at least about 55% (w/w) ethylene glycol.
117. The method of any one of embodiments 1 to 116, wherein eluting comprises contacting the affinity resin with an elution buffer comprising about 50 to 200 mM sodium acetate, 0.05% to 0.2% (w/w) polysorbate 80, and wherein the elution buffer is at a pH of about 5.5 to 6.5.
118. The method of any one of embodiments 1 to 117, wherein eluting comprises contacting the affinity resin with an elution buffer comprising 30 to 80 mM glycine, 5 to 20 mM histidine, 50 to 200 mM NaCl, 3 to 8% (w/w) trehalose, and 0.001 to 0.1% (w/w) polysorbate 80, and wherein the elution buffer is at a pH of 6.5 to 7.5.
119. The method of embodiment 118, wherein eluting further comprises

(a) contacting the affinity resin with a first elution buffer comprising 50 to 200 mM sodium acetate, 0.05% to 0.2% (w/w) polysorbate 80, and wherein the first elution buffer is at a pH of about 5.5 to 6.5;

(b) contacting the affinity resin with a second elution buffer comprising from 30 to 80 mM glycine, 5 to 20 mM histidine, 50 to 200 mM NaCl, 3 to 8% (w/w) trehalose, and 0.001 to 0.1% (w/w) polysorbate 80, and wherein the elution buffer is at a pH of 6.5 to 7.5; and

(c) contacting the affinity resin with a third elution buffer comprising from 50 to 200 mM NaCl and 30 to 80 mM TrisHCl, and wherein the third elution buffer is at a pH of 8.0 to 9.0.

120. The method of any one of embodiments 1 to 119, wherein the steps are performed in succession.
121. The method of any one of embodiments 1 to 120, wherein eluting comprises contacting the affinity resin with an elution buffer comprising about 2 mM magnesium chloride, about 50 mM Arginine-HCl, about 750 mM to about 1000 mM NaCl and at least about 50% (w/w) glycerol at a pH of at least about 8.0.
122. The method of any one of embodiments 1 to 121, wherein eluting comprises contacting the affinity resin with an elution buffer comprising about 2 mM magnesium chloride, about 50 mM Taurine, about 600 mM to about 1000 mM NaCl, about 0.05 to about 0.2% (w/w) octylglycopyranoside, and about 60% (w/w) ethylene glycol at a pH of at least about 7.8.
123. The method of embodiment 122, wherein eluting further comprises
(a) contacting the affinity resin with a fifth buffer comprising from about 20 to about 100 mM Tris-HCl and from about 75 to about 250 mM NaCl, and wherein the fifth buffer has a pH from about 8.0 to about 8.8; and
(b) contacting the affinity resin with a second elution buffer comprising about 1 M ammonium sulfate, about 50 mM Tris HCl, and about 50% (w/w) ethylene glycol at a pH of at least about 6.8.
124. The method of embodiment 123, wherein the steps are performed in succession.
125. The method of any one of embodiments 1 to 124, wherein eluting comprises contacting the affinity resin with an elution buffer comprising about 1 M ammonium sulfate, about 50 mM Tris HCl, and about 50% (w/w) ethylene glycol at a pH of at least about 6.8.
126. The method of any one of embodiments 1 125, wherein eluting comprises contacting the affinity resin with an elution buffer comprising about 20% (w/w) sucrose, about 10% (w/w) sorbitol, about 5% (w/w) mannitol or about 5% (w/w) sucrose, about 15% (w/w) glycerol, about 50 mM Histidine, and about 750 to about 1000 mM NaCl at a pH of at least about 7.8.
127. The method of embodiment 126, wherein eluting further comprises
(a) contacting the affinity resin with a fifth buffer comprising from about 20 to about 100 mM Histidine, from about 80 to about 120 mM NaCl, and wherein the fifth buffer has a pH from about 8.0 to about 8.8; and
(b) contacting the affinity resin with a second elution buffer comprising from about 20 to about 100 mM Histidine, from about 600 to about 900 mM NaCl, and from about 5 to 60% (w/w) DMSO, and wherein the fifth buffer has a pH from about 6.5 to about 8.5.
128. The method of embodiment 127, wherein the steps are performed in succession.
129. The method of any one of embodiments 1 to 128, wherein eluting comprises contacting the affinity resin with an elution buffer comprising about 100 mM Glycine-HCl, about 200 mM NaCl, at a pH of about 2.5.
130. The method of any one of embodiments 109 to 129, wherein the elution buffer is at a pH of about 8.0.
131. The method of any one of embodiments 109 to 129, wherein the elution buffer is at a pH of 8.0.
132. The method of any one of embodiments 109 to 131, wherein eluting comprises a stepwise increase of a counter ion concentration.
133. The method of any one of embodiments 109 to 132, wherein eluting comprises a stepwise increase of an organic solvent concentration.
134. The method of any one of embodiments 1 to 133, wherein the salt in the elution buffer is selected from monovalent, divalent or polyvalent anions, such as chloride, acetate, sulfate, and citrate.
135. The method of any one of embodiments 1 to 134, further comprising elution by applying a gradient of 0 to 100% (w/w) 20-50 mM Hydrochloric acid/800-1200 mM NaCl in 0.5-2.0 mM Hydrochloric acid.
136. The method of any one of embodiments 1 to 135, wherein the AAV obtained from the eluting step has an HC impurity level of s 99.9%.
137. The method of any one of embodiments 1 to 136, wherein the AAV obtained from the eluting step has an HC impurity level of s 99.0%.
138. The method of any one of claims 1 to 137, wherein the AAV obtained from the eluting step has a purity level of 99.0% or greater.
139. The method of any one of claims 1 to 138, wherein the AAV obtained from the eluting step has a purity level of 99.9% or greater.
140. The method of any one of embodiments 1 to 139, wherein at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% of the AAV capsids eluted from the elution step (c) are full AAV capsids.
141. The method of any one of embodiments 1 to 140, wherein the affinity resin is AAVx resin.
142. The method of any one of embodiments 1 to 141, wherein the AAV is AAV9.
143. The method of embodiment 142, wherein the AAV9 comprises a peptide comprising the sequence of SEQ ID NO: 1, SEQ ID NO: 2, and/or SEQ ID NO: 3.
144. The method of any one of embodiments 1 to 143, wherein the method further comprises contacting the AAV containing solution with a filter comprising positively charged groups effective to deplete acidic charged contaminants from the AAV containing solution.
145. The method of any one of embodiments 1 to 144, further comprising nanofiltration of an AAV fraction to remove viruses greater than 35 nm.
146. The method of any one of embodiments 1 to 145, further comprising a polish step comprising performing cation exchange chromatography.
147. The method of any one of embodiments 1 to 146, further comprising testing an AAV fraction via an AAV-specific ELISA.
148. The method of embodiment 147, wherein the AAV specific ELISA is a sandwich ELISA specific for AAV.
149. An AAV product produced by a method according to any one of embodiments 1 to 148.

The following examples are given merely to illustrate the present invention and not in any way to limit its scope.

### Example 1

The following example describes an exemplary method of transfecting a HEK293 cell line with a triple plasmid system to produce rAAV particles comprising a nucleic acid encoding a protein of interest.

Adherent HEK293 cells were grown in suspension conditions in a commercially-available culture medium that is chemically-defined and free of animal-derived components, protein and serum, for example as described in paragraphs [00146]-[00150] of WO2018128688, which is incorporated herein by reference for all intended purposes. The cells were transfected with three plasmids: (1) a helper plasmid, which provides helper viral functions essential for a productive AAV infection, (2) the repcap-plasmid, which carries all information regarding capsid generation, replication and packaging of the virus, and (3) a plasmid containing the gene of interest (GOI), which is packaged into the resulting rAAV particle. The size of the GOI was 2.6 to 3.0 kB. The rAAV particles carrying the gene of interest are in the HEK293 cell line over a period of 3-5 days post-transfection.

The supernatant of a transfected HEK293 cell culture was harvested for example as described in paragraphs [00151]-[00155], Table 1 and Table 2 of WO2018128688, which is incorporated herein by reference for all intended purposes. The harvested supernatant was concentrated and conditioned (diafiltered) for example as described in paragraphs [00156]-[00160], Table 3 and Table 4 of WO2018128688, which is incorporated herein by reference for all intended purposes. Negative chromatography was performed on the diafiltered concentrate for example as described in paragraphs [00161]-[00165] and Table 5 of WO2018128688, which is incorporated herein by reference for all intended purposes.

### Example 2

AAV9 production is developed in a HEK293 cell line after transfection with a triple plasmid system containing encoding cDNA of the protein of interest and VP1, VP2 and VP3 of AAV9. The AAV9 contains vector DNA of approximately 2.6 to 3.0 kB. The clarified cell free culture supernatant is concentrated and diafiltrated with Pall Omega T-Series Cassette 100 kDa. The viral particles are loaded onto a membrane adsorber (MustangQ; Pall Part Number XT140MSTGQP05) at nonbinding conditions, i.e. in a solution comprising 125 mM NaCl and 50 mM TrisHCl at pH 8.5. A pH conditioned LOAD is obtained by adjusting the AAV9 containing flow through to a pH range between 7.4 and 7.8 with 25% HCl.

The following procedure is undertaken. First, a column containing POROS™ CaptureSelect™ AAVX Affinity Resin (Cat. No. 36742; Thermo Fisher) ID 16 mm, with a bed height of 50±0.5 mm, an area of 2.01 cm², and a volume of approximately 10 ml, is activated with five column volumes of a buffer comprising 100 mM glycine, 200 mM NaCl, at a pH of 2.0. The column is then equilibrated with at least five column volumes of 50 mM TrisHCl and 125 mM NaCl at pH 8.5. The pH conditioned LOAD is applied onto the column containing POROS™ CaptureSelect™ AAVX Affinity Resin.

The column is then washed with five column volumes of Wash 1 (W1): 50 mM TrisHCl and 125 mM NaCl, at pH 8.5 and at room temperature (18-26° C.). The column is then washed with five column volumes of Wash 2 (W2): 100 mM sodium acetate and 0.1% Tween 80 (i.e., polysorbate 80), at pH 6.0 and at room temperature (18-26° C.). The column is next washed with five column volumes of Wash 1 (W1): 50 mM TrisHCl and 125 mM NaCl, at pH 8.5 and at room temperature (18-26° C.). The column is then washed with five column volumes of Wash 2 (W2): 100 mM sodium acetate and 0.1% Tween 80, at pH 6.0 and at room temperature (18-26° C.). For all of these wash steps, the linear flow rate is 60 cm/h.

Elution is undertaken by applying five column volumes of W2 above, but at a lower temperature of between +2 to +8° C. Five column volumes of the following secondary elution buffer (ELT-buffer) is then applied to the column at the temperature of between +2 to +8° C.: 50 mM Glycine, 10 mM Histidine, 100 mM NaCl, 5% Trehalose, 0.003% Crillet™ 4 HP (i.e., polysorbate 80), pH 7.0. Five column volumes of the following elution buffer is then applied to the column at the temperature of between +2 to +8° C.: 50 mM TrisHCl and 125 mM NaCl, at pH 8.5. The linear flow rate for these elution steps is 5 cm/h. Five column volumes of purified water is then applied to the column, again at a temperature of between +2 to +8° C. Gradient elution is then performed. Fifteen column volumes of a gradient from 1 mM to 20 mM HCl in purified water is applied at a linear flow rate of 15 cm/h and at a temperature of between +2 to +26° C. to clear the column.

The above procedure is described in more detail in Table 2, with “CV” indicating the number of column volumes of solution added in the step.

TABLE 2

| Step | | Buffer | Buffer comp. | CV | Flow rate | Temp |
|---|---|---|---|---|---|---|
| 1 | Activation | REG2 | 100 mM Glycine, 200 mM | 5 | 60 cm/h | +18-26° C. |
| | | | NaCl | | | |
| | | | pH 2.0 | | | |
| 2 | Equilibration | W1 | 125 mM NaCl/50 mM | ≥5 | 60 cm/h | +18-26° C. |
| | | | TrisHCl | | | |
| | | | pH 8.5 | | | |
| 3 | Product load | Conditioned | — | x | 60 cm/h | +18-26° C. |
| | | AAV | | | | |
| | | containing | | | | |
| | | solution | | | | |
| 4 | Wash 1 | W1 | 125 mM NaCl/50 mM | 5 | 60 cm/h | +18-26° C. |
| | | | TrisHCl | | | |
| | | | pH 8.5 | | | |
| 5 | Wash 2 | W2 | 100 mM NaAcetate/ | 5 | 60 cm/h | +18-26° C. |
| | | | 0.1% Tween 80 | | | |
| | | | pH 6.0 | | | |
| 6 | Wash 3 | W1 | 125 mM NaCl/50 mM | 5 | 60 cm/h | +18-26° C. |
| | | | TrisHCl | | | |
| | | | pH 8.5 | | | |
| 7 | Wash 4 | W2 | 100 mM NaAcetate/ | 5 | 60 cm/h | +18-26° C. |
| | | | 0.1% Tween 80 | | | |
| | | | pH 6.0 | | | |
| 8 | Elution 1 | W2 | 100 mM NaAcetate/ | 5 | 5 cm/h | +2-8° C. |
| | | | 0.1% Tween 80 | | | |
| | | | pH 6.0 | | | |
| 9 | Elution 2 | ELT | 50 mM Glycine,10 mM | 5 | 5 cm/h | +2-8° C. |
| | | | Histidine,100 mM | | | |
| | | | NaCl, 5% Trehalose, | | | |
| | | | 0.003% Crillet 4 HP, pH | | | |
| | | | 7.0 (ELT-buffer) | | | |
| 10 | Elution 3 | W1 | 125 mM NaCl/50 mM | 5 | 5 cm/h | +2-8° C. |
| | | | TrisHCl | | | |
| | | | pH 8.5 | | | |
| 11 | Wash 5 | PW | Purified water | 5 | 5 cm/h | +2-8° C. |
| 12 | Elution 4 | Gradient | Gradient from 1 mM to | 15 | 20 cm/h | +2-26° C. |
| | | | 20 mM HCl/200 mL | | | |
| | | | NaCl in | | | |
| | | | Purified water | | | |

Methods for cooling the column from room temperature to about +2-8° C. includes:
Chromatography skid AKTA pure 150 in a cooling cabinet Unichromat 1500;
Chromatography columns with temperature jacket (Water cooling);
Cold buffers used for elution; or
A combination of above.

The samples taken are assayed by each of ITR qPCR, ELISA against AAV antigens and ELISA against HEK293 HCP to assess yield and whether losses may have occurred in the steps.

ELISA is used to measure the quantity of AAV9 antigen. ELISA is carried out with an AAV-9 titration ELISA Kit (Art. No. PRAAV9; Progen (Heidelberg, Germany) on a TECAN Roboter system. Briefly, a monoclonal antibody specific for AAV9 capsids (AAV8/9 antibody (“ADK8/9 antibody”, Cat. No. 03-651161, American Research Products, Inc., Waltham, Mass.)) is coated onto microtiter strips and is used to capture AAV9 particles from the AAV fraction. The capture AAV9 particles are detected by two steps. In a first step, a biotin-conjugated monoclonal antibody specific for the ADK8/9 antibody is bound to the immune complex (of ADK8/9 and ADK8/9 antibody). Streptavidin peroxidase conjugates are added to the immune complexes bound to the biotin-conjugated monoclonal antibody and the streptavidin peroxidase conjugates react with the biotin. A peroxidase substrate solution is added and a color reaction which is proportional to the amount of bound AAV particles occurs. The color reaction is measured photometrically at 450 nm.

An ITR-qPCR assay is used to determine the genome copy titer by quantifying the inverted tandem repeats found in the vector encoding for the gene of interest (e.g., human Factor VIII or human Factor IX). HEK-HCP is a measurement of the residual host cell protein by ELISA. LDH is determined by a colorimetric activity assay.

In the in-vitro biopotency assay, the viral vector AAV9 infects a hepatic target cell line, which subsequently secretes functional, measurable encoded protein into the medium. In a first step HepG2 target cells are transduced infected by AAV9. During incubation time encoded protein is released into cell supernatant. In a second step the activity of the encoded protein into the cell culture supernatant is directly measured by an activity assay. The measurement of an AAV9 sample is given as a percentage relative to a reference material. The method allows a quantitative assessment of the biologic function of the AAV9 gene therapy vector.

SDS-PAGE analysis is performed to determine if there was a reduction in Heat Shock Protein 70 kDa (HSP70) when using the test procedure with the wash steps instead of the comparative procedure. A Western Blot is performed using an Anti-Hsp70 antibody (Abcam, catalog no. ab79852) as the primary antibody at 1:2000 dilution for two hours, and goat anti-rabbit igG (H+L) AP conjugate as the secondary antibody (Sigma, catalog no. A8025) in 1:1000 dilution for one hour.

An SDS-PAGE silver stain assay is performed to determine the overall level of impurities present. Analytical ultracentrifugation (AUC) is performed to quantify the amount of AAV9 present, to determine the relative amount of full capsids, empty capsids, and those that have additional DNA as compared to a full capsid, i.e. overfilled.

A Western Blot with 12% anti-AAV antibody is performed to determine the levels and purity of the AAV9 recovered after purification according to the test and comparative procedures. The Western blot is performed with monoclonal antibodies to VP1, VP2 and VP3 of AAV9 as the primary antibodies, with goat anti-mouse ALP antibody (Sigma, catalog number A4656) as the secondary antibody.

These assays are summarized in Table 3 below.

TABLE 3:

| | | Fraction | Sample | Storage and |
|---|---|---|---|---|
| | | (BDS, UC | Volume | Shipping |
| Priority | Test | or both) | TMAE | conditions |
| 1 | AAV9 ELISA | Selected | 0.2 mL | ≤−60° C. |
| | | fractions | | |
| 2 | Analytical | Selected | 0.5 mL | ≤−60° C. |
| | Ultracentrifugation (AUC) | fractions | | |
| 3 | GOI titer (ITR-qPCR) | Selected | 0.2 mL | ≤−60° C. |
| | | fractions | | |
| 4 | AAV9 agarose gel DNA | Selected | 0.2 mL | ≤−60° C. |
| | analysis by native/alkaline | fractions | | |
| | agarose gel electrophoresis | | | |
| 5 | SDS PAGE (8%, WB, | Selected | 0.1 mL | ≤−60° C. |
| | silver stain) | fractions | | |
| 6 | Biopotency | Selected | 0.2 mL | ≤−60° C. |
| | | fraction | | |
| Total | | | 1.4 mL | |

### Example 3

AAV9 production was developed in a HEK293 cell line after transfection with a triple plasmid system containing encoding cDNA of the protein of interest and VP1, VP2 and VP3 of AAV9. The AAV9 contains vector DNA of approximately 2.6 to 3.0 kB. The clarified cell free culture supernatant was concentrated and diafiltrated with Pall Omega T-Series Cassette 100 kDa. The viral particles were loaded onto a membrane adsorber (MustangQ; Pall Part Number XT140MSTGQP05) at nonbinding conditions, i.e., in a solution comprising 125 mM NaCl and 50 mM TrisHCl at pH 8.5. A pH conditioned LOAD was obtained by adjusting the AAV9 containing flow through to a pH range between 8.2 and 8.7 with 25% HCl.

The following procedure was undertaken. Note that all buffers disclosed in this example were made at room temperature and the pH of all buffers were measured at room temperature. First, a column containing POROS™ CaptureSelect™ AAVX Affinity Resin (Cat. No. 36742; Thermo Fisher) ID 16 mm, with a bed height of 50±0.5 mm, an area of 2.01 cm², and a volume of approximately 10 ml, was activated with five column volumes of 100 mM glycine, 200 mM NaCl, at a pH of 2.0. The column was then equilibrated with at least 10 column volumes of 50 mM TrisHCl and 125 mM NaCl at pH 8.5. The pH conditioned LOAD was applied onto the column containing POROS™ CaptureSelect™ AAVX Affinity Resin.

The column was then washed with five column volumes of Wash 1 (W1): 50 mM TrisHCl and 125 mM NaCl, at pH 8.5 and at room temperature (18-26° C.). The column was then washed with five column volumes of Wash 2 (W2): 100 mM sodium acetate and 0.1% Tween 80, at pH 6.0 and at room temperature (18-26° C.). The column was next washed with five column volumes of Wash 1 (W1): 50 mM TrisHCl and 125 mM NaCl, at pH 8.5 and at room temperature (18-26° C.). For all of these wash steps, the linear flow rate was 60 cm/h.

Elution was undertaken by applying 10 column volumes of W1 above, but at a lower temperature of between +2 to +8° C. For the elution (i.e., step 7 in Table 4 below), chromatography skid, column, and buffers were all lowered to below +8° C. via placing all items in a cooling cabinet (Unichromat 1500). The linear flow rate for elution was 5 cm/h. The column was then stripped with five column volumes of 100 mM glycine, 200 mM NaCl, at a pH of 2.0.

The above procedure is described in more detail in Table 4, with “CV” indicating the number of column volumes of solution added in the step.

TABLE 4

| | | | | Amount | | |
|---|---|---|---|---|---|---|
| Step | | Buffer | Buffer comp. | [CV] | Flow rate | Temp |
| 1 | Activation | REG2 | 100 mM Glycine, 200 mM | 5 | 60 cm/h | +18-26° C. |
| | | | NaCl | | | |
| | | | pH 2.0 | | | |
| 2 | Equilibration | W1 | 125 mM NaCl/50 mM | 10 | 60 cm/h | +18-26° C. |
| | | | TrisHCl | | | |
| | | | pH 8.5 | | | |
| 3 | Product load | Conditioned | 125 mM NaCl/50 mM | — | 60 cm/h | +18-26° C. |
| | | AAV | TrisHCl | | | |
| | | containing | pH 8.5 | | | |
| | | solution | | | | |
| 4 | Wash 1 | W1 | 125 mM NaCl/50 mM | 5 | 60 cm/h | +18-26° C. |
| | | | TrisHCl | | | |
| | | | pH 8.5 | | | |
| 5 | Wash 2 | W2 | 100 mM NaAcetate/ | 5 | 60 cm/h | +18-26° C. |
| | | | 0.1% Tween 80 | | | |
| | | | pH 6.0 | | | |
| 6 | Wash 3 | W1 | 125 mM NaCl/50 mM | 5 | 60 cm/h | +18-26° C. |
| | | | TrisHCl | | | |
| | | | pH 8.5 | | | |
| 7 | Elution | W1 | 125 mM NaCl/50 mM | 10 | 5 cm/h | +2-8° C. |
| | | | TrisHCl | | | |
| | | | pH 8.5 | | | |
| 8 | Wash | 4 | 150 mM NaCl | 5 | 60 cm/h | +2-8° C. |
| | | | 20 mM TrisHCl | | | |
| | | | TBS 0.1% Tween80 | | | |
| | | | pH 7.4 | | | |
| 9 | STRIP | REG2 | 100 mM Glycine, 200 mM | 5 | 15 cm/h | +2-8° C. |
| | | | NaCl | | | |
| | | | pH 2.0 | | | |

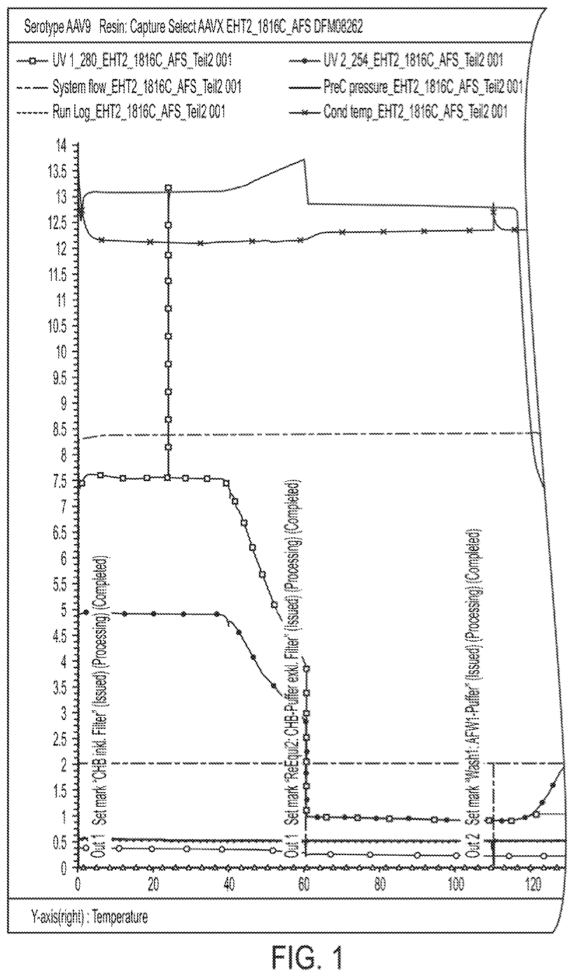

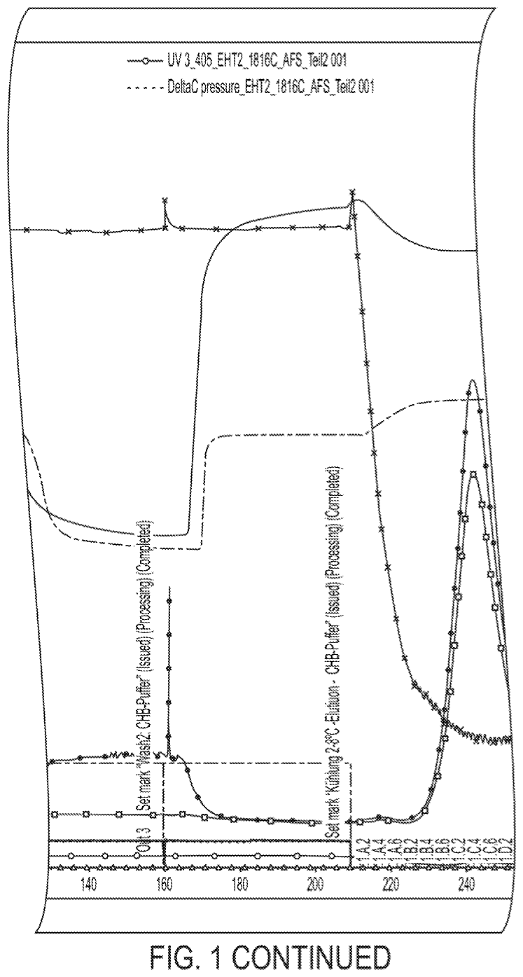

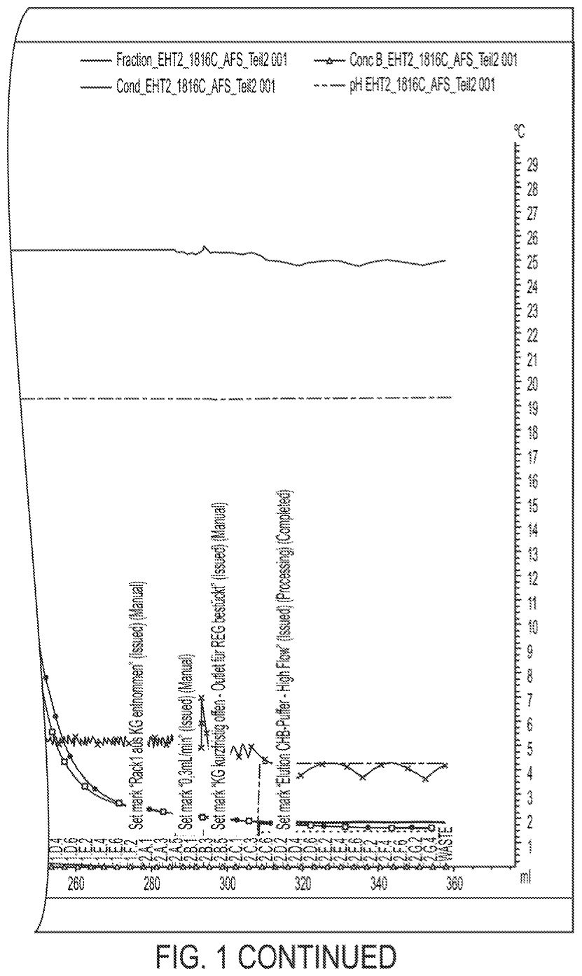

The chromatogram from the above procedure is shown in FIG. 1. The results from the protocol of Table 4 are shown in Table 5 below. The E1 Pool “low temperature eluate” values reflect the amounts after step 7 of Table 4 above. The “strip” values reflect the amounts after step 9 of Table 4 above. Table 5 clearly demonstrates that elution is caused by the shift in the temperature to +2-+8° C. as W1 and W3, which did not result in a significant elution of AAV9, were conducted with the same buffer (i.e., 125 mM NaCl and 50 mM at pH 8.5) but at room temperature (i.e., +18-25° C.).

TABLE 5

| | | | | | | | AAV9 | | |
|---|---|---|---|---|---|---|---|---|---|
| | | | | | | AAV9 | Total | | Ratio |
| | | | qPCR | Total | | Antigen | Antigen | | qPCR/ |
| | Temp. | Amount | vg/ml × | qPCR | qPCR | cp/ml × | cp × | Antigen | Antigen |
| | [° C.] | [g] | 10¹¹ | vg × 10¹¹ | [%] | 10¹¹ | 10¹¹ | [%] | [vg/cp] |
| LOAD | +22-28 | 1006.60 | 4.12 | 4147.19 | 100.0 | 6.235 | 6276.4 | 100.00 | 0.661 |
| FT/W1 | +22-28 | 1135.17 | <LOQ | — | — | 0.0975 | 110.68 | 1.76 | — |
| W2 | +22-28 | 52.38 | <LOQ | — | — | 0.046 | 2.4 | 0.04 | — |
| W3 | +22-28 | 51.81 | <LOQ | — | — | 0.046 | 2.38 | 0.04 | — |
| E1 Pool | +2-8 | 48.93 | 64.20 | 3141.31 | 75.75 | 57.50 | 2813.48 | 44.83 | 1.117 |
| Strip | +2-8 | 52.80 | 2.11 | 111.41 | 2.69 | 20.1 | 1061.28 | 16.91 | 0.105 |

LOQ: Below detection limit;
— : Could not be calculated because the amount of DNA was below detection limits;
E1 Pool is the Eluate

Furthermore, the measured impurity content for the process described in Table 4 is shown below in Table 6. Impurity samples were taken for the initial sample and again after step 7.

TABLE 6

| | | HEK293 | Total | |
|---|---|---|---|---|
| | Amount | HCP | HEK293 | |
| | [g] | μg/ml | HCP | [%] |
| LOAD | 1135.17 | 541.6 | 545174.56 | 100.00 |
| E1 Pool | 48.93 | 1.724 | 84.36 | 0.02 |

Table 7 examines the % full capsids and % overfilled capsids, which were determined based on the particle size. As used herein, particle sizes are given in a range of Svedbergs, which is based on the sedimentation rate of the particle.

TABLE 7

| | % | % | % | |
|---|---|---|---|---|
| | Empty | Under | Full | % |
| | Capsids | Populated | Capsids | Overfilled |
| | S | S | S | S |
| Run Setup | 60 to 65 | 70 to 74 | 86 to 90 | 99 to 104 |
| E1 Pool | 1.9 | 7.1 | 87.5 | 3.4 |
| STRIP | 71.1 | 6.7 | 11.3 | 10.9 |

As used herein, the term “yield qPCR” refers to the percentage of qPCR present compared to the initial amount of qPCR in the LOAD. As used herein, the term “yield Antigen” refers to the percentage of AAV9 present compared to the initial amount of AAV9 in the LOAD. The 75.8% yield as measured by qPCR (Table 5) and the 87.5% of AAV9 with full capsids (Table 7) indicates that the low temperature eluate protocol described in this example provides for substantial enrichment of AAV9 full capsids.

The chromatogram data from the above procedure was compared to a standard elution procedure (100 mM Glycine and 200 mM NaCl at pH 2.7 as shown in Table 8), and the results are shown below in Table 9. Starting materials were the same for both runs.

TABLE 8

| | | | | Amount | | |
|---|---|---|---|---|---|---|
| Step | | Buffer | Buffer comp. | [CV] | Flow rate | Temp |
| 1 | Activation | REG2 | 100 mM Glycine, | 5 | 60 cm/h | +18-26° C. |
| | | | 200 mM NaCl | | | |
| | | | pH 2.0 | | | |
| 2 | Equilibration | W1 | 125 mM NaCl/50 mM | 10 | 60 cm/h | +18-26° C. |
| | | | TrisHCl | | | |
| | | | pH 8.5 | | | |
| 3 | Product load | Conditioned | 125 mM NaCl/50 mM | — | 60 cm/h | +18-26° C. |
| | | AAV9 | TrisHCl | | | |
| | | containing | pH 8.5 | | | |
| | | solution | | | | |
| 4 | Wash 1 | W1 | 125 mM NaCl/50 mM | 5 | 60 cm/h | +18-26° C. |
| | | | TrisHCl | | | |
| | | | pH 8.5 | | | |
| 5 | Wash 2 | W2 | 100 mM NaAcetat/ | 5 | 60 cm/h | +18-26° C. |
| | | | 0.1% Tween 80 | | | |
| | | | pH 6.0 | | | |
| 6 | Wash 3 | W1 | 125 mM NaCl/50 mM | 5 | 60 cm/h | +18-26° C. |
| | | | TrisHCl | | | |
| | | | pH 8.5 | | | |
| 7 | Elution | REG | 100 mM Glycine | 10 | 30 cm/h | +18-26° C. |
| | | | 200 mM NaCl | | | |
| | | | pH 2.7 | | | |

TABLE 9

| | % | % | % | |
|---|---|---|---|---|
| | Empty | Under | Full | % |
| | Capsids | Populated | Capsids | Overfilled |
| | S | S | S | S |
| Run Setup | 60 to 65 | 70 to 74 | 86 to 90 | 99 to 104 |
| STANDARD | 18.8 | 7.1 | 71.1 | 3.0 |
| Elution | | | | |
| COLD Elution | 1.9 | 7.1 | 87.5 | 3.4 |

Tables 10 and 11 provide additional data from the standard elution procedure of Table 8.

TABLE 10

| | | | | | | AAV9 | | |
|---|---|---|---|---|---|---|---|---|
| | | | | | AAV9 | Total | | Ratio |
| | | qPCR | Total | | Antigen | Antigen | AAV9 | qPCR/ |
| | Amount | [vg/ml | qPCR | qPCR | [cp/ml × | [cp/ml × | Antigen | Antigen |
| | [g] | ×10¹¹] | [vg × 10¹¹] | [%] | 10¹¹ | 10¹¹] | [%] | [vg/cp] |
| LOAD | 6049 | 2.79 | 16861.59 | 100 | 5.76 | 34842.24 | 100 | 0.48 |
| ELUTION | 136.87 | 142.5 | 19503.29 | 115.67 | 207.5 | 28399.53 | 81.51 | 0.69 |

TABLE 11

| | | | Total | |
|---|---|---|---|---|
| | | HEK293 | HEK293 | |
| | Amount | HCP | HCP | |
| | [g] | [μg/ml] | [μg] | [%] |
| LOAD | 6049 | 528.4 | 3196291.60 | 100 |
| ELUTION | 136.87 | 1.872 | 256.21 | 0.01 |

As demonstrated in Table 5, AAV9 binds on the CaptureSelect™ AAVx resin at room temperature (i.e., about 20-28° C.) and it was surprisingly and unexpectedly found the bound AAV9 can then be eluted with a temperature shift from above +18° C. to below +8° C. in the same buffer system. Thus, the temperature shift protocol has the benefit of a mild elution at a low temperature to help preserve the structure and infectivity of the AAV particles. Likewise, use of a mild elution buffer can be easily implemented in a manufacturing environment and more efficient as there is no need for a buffer change for the elution step. Moreover, it was advantageously found that Full AAV9 capsids elute by lowering the temperature, while the empty and unspecific AAV9 vectors remained bound until they were eluted with the step strip-acidic elution (see Table 7).

### Example 4

Example 3 demonstrated elution of AAV9 from CaptureSelect™ AAVx using a buffer of 125 mM NaCl and 50 mM TrisHCl, pH 8.5 with a temperature shift from above +18° C. to below +8° C. This example was performed to examine the potential of alternative buffer systems for elution of AAV9 from AAVx with a temperature shift from above +18° C. to below +8° C. After loading AAV9 onto an AAVx resin the buffers were applied first at the higher temperature range as wash buffers and afterwards at lower temperature as elution buffers.

AAV9 production was developed in a HEK293 cell line after transfection with a triple plasmid system containing encoding cDNA of the protein of interest and VP1, VP2 and VP3 of AAV9. The AAV9 contains vector DNA of approximately 2.6 to 3.0 kB. The clarified cell free culture supernatant was concentrated and diafiltrated with Pall Omega T-Series Cassette 100 kDa. The viral particles were loaded onto a membrane adsorber (MustangQ; Pall Part Number XT140MSTGQP05) at nonbinding conditions, i.e., in a solution comprising 125 mM NaCl and 50 mM TrisHCl at pH 8.5. A pH conditioned LOAD was obtained by adjusting the AAV9 containing flow through to a pH range between 8.2 and 8.7 with 25% HCl.

The following procedure was undertaken. Note that all buffers disclosed in this example were made at room temperature and the pH of all buffers were measured at room temperature.

Three buffers with increasing pH and conductivity were applied at higher temperature (+18 to +28° C.) at first to confirm that AAV9 is still bound onto the ligand. After cooling down the column to +2 to +8° C., wherein the chromatography skid, column, and buffers were all lowered to below +8° C. via placing all items in a cooling cabinet (Unichromat 1500), then the same buffers were applied to examine the elution properties. First, a column containing POROS™ CaptureSelect™ AAVX Affinity Resin (Cat. No. 36742; Thermo Fisher) ID 11 mm, with a bed height of 57 mm, an area of 0.95 cm2, and a volume of approximately 5.4 ml, was activated with five column volumes of a buffer comprising 100 mM glycine, 200 mM NaCl, at a pH of 2.0. The column was then equilibrated with at least five column volumes of 50 mM TrisHCl and 125 mM NaCl at pH 8.5. The pH conditioned LOAD was applied onto the column containing POROS™ CaptureSelect™ AAVX Affinity Resin.

The column was then washed with five column volumes of Wash 1 (W1): 50 mM TrisHCl and 125 mM NaCl, at pH 8.5 and at room temperature (18-26° C.). The column was then washed with five column volumes of Wash 2 (W2): 100 mM sodium acetate and 0.1% Tween 80, at pH 6.0 and at room temperature (18-26° C.). The column was next washed with five column volumes of Wash 1 (W1): 50 mM TrisHCl and 125 mM NaCl, at pH 8.5 and at room temperature (18-26° C.). The column was then washed with five column volumes of Wash 2 (W2): 100 mM sodium acetate and 0.1% Tween 80, at pH 6.0 and at room temperature (18-26° C.). For all of these wash steps, the linear flow rate was 60 cm/h.

Elution was first undertaken by applying five column volumes of W2 above, but at a lower temperature of between +2 to +8° C. at a linear flow rate of 5 cm/h. Five column volumes of the following secondary elution buffer were then applied to the column at the temperature of between +2 to +8° C.: 50 mM Glycine, 10 mM Histidine, 100 mM NaCl, 5% Trehalose, 0.005% Crillet 4 HP, pH 7.0 (ELT-buffer). Five column volumes of the following elution buffer were then applied to the column at the temperature of between +2 to +8° C.: 50 mM TrisHCl and 125 mM NaCl, at pH 8.5. The linear flow rate for these elution steps was 30 cm/h. Five column volumes of purified water were then applied to the column, again at a temperature of between +2 to +8° C. Gradient elution was then performed. 15 column volumes of a gradient from 1 mM to 20 mM HCl, 200 mM NaCl in purified water was applied at a linear flow rate of 20 cm/h and at a temperature of between +2 to +8° C.

Column cleaning was performed by applying five column volumes of the following regeneration buffer: 20 mM HCl, 200 mM NaCl in purified water followed by five column volumes of 50 mM TrisHCl and 125 mM NaCl, at pH 8.5 with 60 cm/h followed by five column volumes of 1 M L-Arginine monohydrochlorid+200 mM NaCl with a linear flow rate of 30 cm/h at a temperature of between +2 to +8° C.

The above procedure is described in more detail in Table 12 with “CV” indicating the number of column volumes of solution added in the step.

TABLE 12

| | | | | Amount | | |
|---|---|---|---|---|---|---|
| | Step | Buffer | Buffer comp. | (CV) | Flow rate | Temp |
| 1 | Activation | REG2 | 100 mM Glycine, | 5 | 60 cm/h | +18-26° C. |
| | | | 200 mM NaCl | | | |
| | | | pH 2.0 | | | |
| 2 | Equilibration | W1 | 125 mM NaCl/50 mM | 10 | 60 cm/h | +18-26° C. |
| | | | TrisHCl | | | |
| | | | pH 8.5 | | | |
| 3 | Product load | Conditioned | 125 mM NaCl/50 mM | — | 60 cm/h | +18-26° C. |
| | | AAV9 | TrisHCl | | | |
| | | containing | pH 8.5 | | | |
| | | solution | | | | |
| 4 | Wash 1 | W1 | 125 mM NaCl/50 mM | 5 | 60 cm/h | +18-26° C. |
| | | | TrisHCl | | | |
| | | | pH 8.5 | | | |
| 5 | Wash 2 | W2 | 100 mM NaAcetat/ | 5 | 60 cm/h | +18-26° C. |
| | | | 0.1% Tween 80 | | | |
| | | | pH 6.0 | | | |
| 6 | Wash 3 | W1 | 125 mM NaCl/50 mM | 5 | 60 cm/h | +18-26° C. |
| | | | TrisHCl | | | |
| | | | pH 8.5 | | | |
| 7 | Wash 4 | W2 | 100 mM NaAcetat/ | 5 | 60 cm/h | +18-26° C. |
| | | | 0.1% Tween 80 | | | |
| | | | pH 6.0 | | | |
| 8a | Elution 1.1 | W2 | 100 mM NaAcetat/ | 5 | 5 cm/h | +2-8° C. |
| | Cool down | | 0.1% Tween 80 | | | |
| | | | pH 6.0 | | | |
| 8b | Elution 1.2 | W2 | 100 mM NaAcetat/ | 5 | 30 cm/h | +2-8° C. |
| | | | 0.1% Tween 80 | | | |
| | | | pH 6.0 | | | |
| 9 | Elution 2 | ELT | 50 mM Glycin,10 mM | 5 | 30 cm/h | +2-8° C. |
| | | | Histidine,100 mM | | | |
| | | | NaCl, 5% Trehalose, | | | |
| | | | 0.005% Crillet 4 HP, pH | | | |
| | | | 7.0 (ELT-buffer) | | | |
| 10 | Elution 3 | W1 | 125 mM NaCl/50 mM | 5 | 30 cm/h | +2-8° C. |
| | | | TrisHCl | | | |
| | | | pH 8.5 | | | |
| 11 | Wash 5 | PW | Purified water | 5 | 30 cm/h | +2-8° C. |
| | | | Gradient from 1 mM to | | | |
| 12 | Elution 4 | Gradient | 20 mM HCl/200 | 15 | 20 cm/h | +2-8° C. |
| | | | mM NaCl in | | | |
| | | | Purified water | | | |
| 13 | Strip 1 | STR1 | 20 mM HCl | 5 | 60 cm/h | +2-8° C. |
| | | | 200 mM NaCl in | | | |
| | | | Purified water | | | |
| 14 | Strip 2 | W1 | 125 mM NaCl/50 mM | 5 | 60 cm/h | +2-8° C. |
| | | | TrisHCl | | | |
| | | | pH 8.5 | | | |
| 15 | Strip 3 | STR3 | 1M L-Arginine | 5 | 30 cm/h | +2-8° C. |
| | | | Monohydrochlorid + | | | |
| | | | 200 mM NaCl (pH native, | | | |
| | | | approx. pH 5.6 | | | |

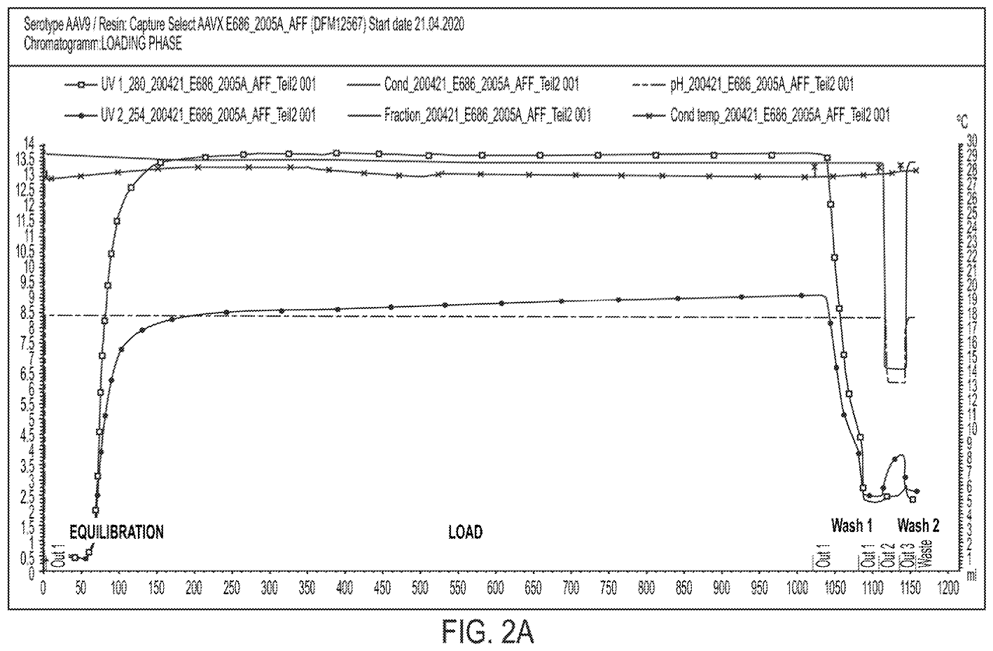

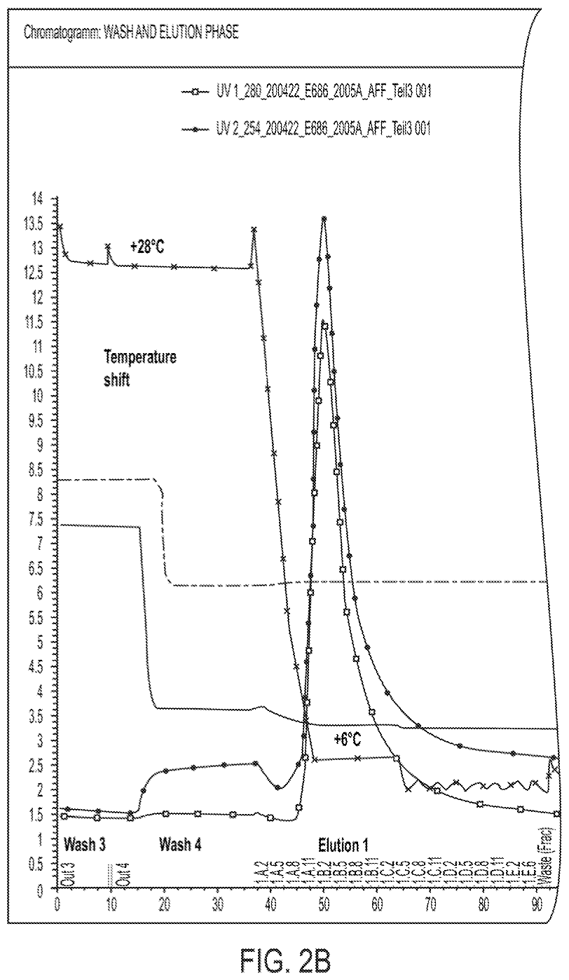

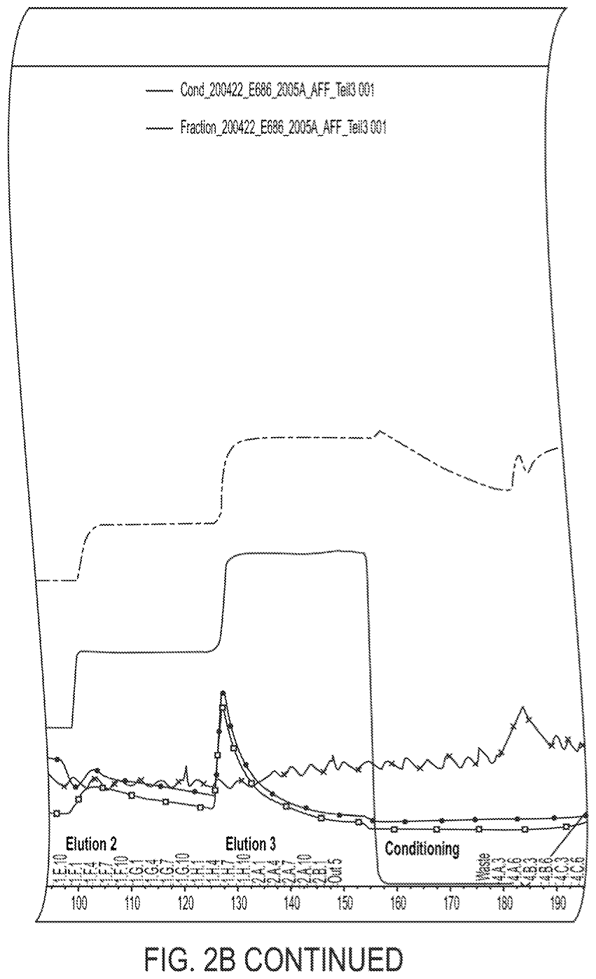

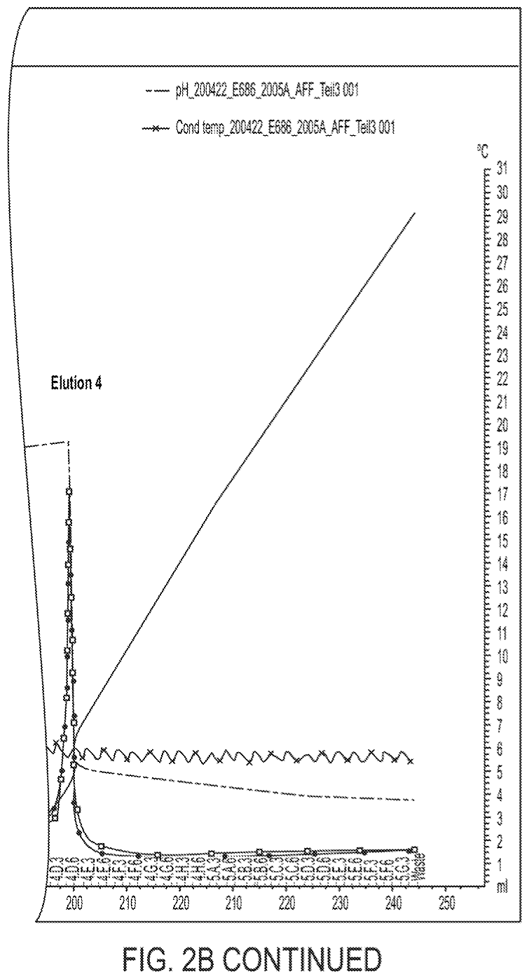

The results from the above procedure, and particularly the percentage yield of AAV9 eluted from the column, are shown below in Table 13. The chromatogram from the above procedure is shown in FIGS. 2A-2B.

TABLE 13

| | | | | | | | AAV9 | | |
|---|---|---|---|---|---|---|---|---|---|
| | | | | Total | | AAV9 | Total | | Ratio |
| | | | ddPCR | ddprR | | Antigen | Antigen | AAV9 | qPCR/ |
| | Temp. | Amount | vg/ml × | vg × | ddPCR | cp/ml × | cp × | Antigen | Antigen |
| | [° C.] | [g] | 10¹¹ | 10¹¹ | [%] | 10¹¹ | 10¹¹ | [%] | vg/cp |
| LOAD | +22-28 | 1004.46 | 1.61 | 1617.18 | 100.00 | 2.86 | 2872.76 | 100.00 | 0.563 |
| FT/W1 | +22-28 | 1106.16 | <LOQ | — | — | <0.038 | <0.000 | — | — |
| W2 | +22-28 | 27.2 | <LOQ | — | — | <0.038 | <0.000 | — | — |
| W3 | +22-28 | 26.24 | <LOQ | — | — | <0.038 | <0.000 | — | — |
| W4 | +22-28 | 27.2 | <LOQ | — | — | <0.038 | <0.000 | — | — |
| E1 | +2-8 | 46.91 | 23.94 | 1122.80 | 69.43 | * | 1758.36 | 61.21 | 0.639 |
| (E1.1 + | | | | | | | | | |
| E1.2)* | | | | | | | | | |
| E2 | +2-8 | 26.28 | 4.02 | 105.65 6.53 | 9.82 | 258.07 | 8.98 | 0.409 | |
| E3 | +2-8 | 21.86 | 4.74 | 103.62 | 6.41 | 15.2 | 332.27 | 11.57 | 0.312 |
| E4 | +2-8 | 4.96 | 7.00 | 34.72 | 2.15 | 31.40 | 155.74 | 5.42 | 0.223 |

LOQ: Below detection limit;
— : Could not be calculated because the amount of DNA was below detection limits;
*: Pool of E1.1 and E1.2-eluate was collected in a main peak of E1.1 and the tailing part of E1.2.

As shown, elution at lower temperature range is also confirmed with a buffer other than 125 mM NaCl, 50 mM Tris HCl, pH 8.5. Furthermore, the measured impurity content for the process described in Table 12 is shown below in Table 14. Impurity samples were taken after each of the elution steps (steps 8a, 8b, 9, 10, and 12).

TABLE 14

| | | HEK293 | Total | |
|---|---|---|---|---|
| | Amount | HCP | HEK293 | |
| | [g] | μg/ml | HCP | [%] |
| LOAD | 1004.46 | 507.5 | 509763.45 | 100.00 |
| E1.1 | 19.83 | 0.424 | 8.41 | 0.002 |
| E1.2 | 27.08 | <0.125 | <3.385 | |
| E2 | 26.28 | 0.144 | 3.78 | 0.001 |
| E3 | 21.86 | <0.125 | <2.733 | |
| E4 | 4.96 | 0.844 | 4.19 | 0.001 |

A comparison of low temperature elution between the buffers of Example 3 and Example 4 are shown below in Table 15.

TABLE 15

| | | | Amount | | Amount | |
|---|---|---|---|---|---|---|
| Step | | Example 4 | [CV] | Example 3 | [CV] | Temp |
| 1 | Activation | 100 mM Glycine, 200 mM | 5 | 100 mM Glycine, 200 mM | 5 | +18-26° C. |
| | | NaCl | | NaCl | | |
| | | pH 2.0 2.0 | | pH 2.0 | | |
| 2 | Equilibration | 125 mM NaCl/50 mM | 10 | 125 mM NaCl/50 mM TrisHCl | 10 | +18-26° C. |
| | | TrisHCl | | pH 8.5 | | |
| | | pH 8.5 | | | | |
| 3 | Product load | 125 mM NaCl/50 mM | — | 125 mM NaCl/50 mM TrisHCl | — | +18-26° C. |
| | | TrisHCl | | pH 8.5 | | |
| | | pH 8.5 | | | | |
| 4 | Wash 1 | 125 mM NaCl/50 mM | 5 | 125 mM NaCl/50 mM TrisHCl | 5 | +18-26° C. |
| | | TrisHCl | | pH 8.5 | | |
| | | pH 8.5 | | | | |
| 5 | Wash 2 | 100 mM NaAcetat/ | 5 | 100 mM NaAcetat/ | 5 | +18-26° C. |
| | | 0.1% Tween 80 | | 0.1% Tween 80 | | |
| | | pH 6.0 6.0 | | pH 6.0 | | |
| 6 | Wash 3 | 125 mM NaCl/50 mM | | 125 mM NaCl/50 mM TrisHCl | | |
| | | TrisHCl | 5 | pH 8.5 | 5 | +18-26° C. |
| | | pH 8.5 | | | | |
| 7 | Wash 4 | 100 mM NaAcetat/ | 5 | x | x | +18-26° C. |
| | | 0.1% Tween 80 | | | | |
| | | pH 6.0 | | | | |
| 8 | Elution 1 | 100 mM NaAcetat/ | 10 | x | x | +2-8° C. |
| | Cool down | 0.1% Tween 80 | | | | |
| | | pH 6.0 | | | | |
| 9 | Elution 2 | 50 mM Glycin, 10 mM | 5 | x | x | +2-8° C. |
| | | Histidine, 100 mM | | | | |
| | | 1 NaCl , 5% Trehalose, | | | | |
| | | 0.005% Crillet 4 HP, pH | | | | |
| | | 7.0 (ELT-buffer) | | | | |
| 10 | Elution 3 | 125 mM NaCl/50 mM | 5 | 125 mM NaCl/50 mM TrisHCl | 10 | +2-8° C. |
| | | TrisHCl | | pH 8.5 | | |
| | | pH 8.5 | | | | |
| 11 | Wash 5 | Purified water | 5 | 150 mM NaCl | 5 | +2-8° C. |
| | | | | 20 mM TrisHCl | | |
| | | | | 0.1% Tween80 | | |
| | | | | pH 7.4 | | |
| 12 | Elution 4 | Gradient from 1 mM to | 15 | x | x | +2-8° C. |
| | | 20 mM HCl/200 mM | | | | |
| | | NaCl in | | | | |
| | | Purified water | | | | |
| 13 | Strip 1 | 20 mM HCl | 5 | x | x | +2-8° C. |
| | | 200 mM NaCl in | | | | |
| | | Purified water | | | | |
| 14 | Strip 2 | 125 mM NaCl/50 mM | 5 | x | x | +2-8° C. |
| | | TrisHCl pH 8.5 | | | | |
| 15 | Strip 3 | 1M L-Arginine | 5 | 100 mM Glycine, 200 mM | 5 | +2-8° C. |
| | | Monohydrochlorid + | | NaCl | | |
| | | 200 mM NaCl (pH nativ, | | pH 2.0 | | |
| | | approx. pH 5.6 | | | | |

TABLE 16

| | EXAMPLE 3 | | | | EXAMPLE 4 | | | |
|---|---|---|---|---|---|---|---|---|
| Buffer | Area % | Area % | Area % | Area % | Area % | Area % | Area % | Area % |
| Composition | Empty | Subpop. | Full | Overfilled | Empty | Subpop. | Full | Overfilled |
| 100 mM | n.a | n.a | n.a | n.a | 11.8 | 0.5 | 77.0 | 10.6 |
| NaAcetat/ | | | | | | | | |
| 0.1% Tween 80 | | | | | | | | |
| pH 6.0 | | | | | | | | |
| 50 mM | n.a | n.a | n.a | n.a | —* | —* | —* | —* |
| Glycin,10 mM | | | | | | | | |
| Histidine, 100 mM | | | | | | | | |
| NaCl, 5% | | | | | | | | |
| Trehalose, | | | | | | | | |
| 0.005% Crillet 4 | | | | | | | | |
| HP, pH 7.0 | | | | | | | | |
| (ELT-buffer) | | | | | | | | |
| 125 mM NaCl/ | 1.9 | 7.1 | 87.5 | 3.4 | 29.5 | 2.0 | 64.1 | 4.3 |
| 50 mM TrisHCl | | | | | | | | |
| pH 8.5 | | | | | | | | |

n.a = does not applied;
.* = not tested as too low of a concentration for AUC analysis.

Elution was confirmed with 100 mM NaAcetate, 0.1% (w/w) polysorbate, pH 6.0 at +2-+8° C. and not at room temperature (see Table 16). Its notable that 100 mM NaAcetate at low temperature is opposite in pH and conductivity to 125 mM NaCl, 50 mM TrisHCl, pH 8.5, as elution efficiency normally increases with increasing pH from pH 6 to pH 8.5. Thus, Example 4 demonstrates that temperature is the driving force for elution and the pH and conductivity have lower impact to the elution than the temperature. It is also confirmed that this affinity method with the temperature shift leads to a higher content of full AAV capsids.

Thus, Example 3 and 4 demonstrates that that the mode of action to elute AAV9 from AAVx depends mainly on lowering the temperature, independent of the buffer system.

### Example 5

Batch absorption of AAV9 was made according to the procedure described below. Two sets with the two different resins above were incubated with an AAV9 containing solution at room temperature first to bind the AAV9 followed by an elution at lower temperature. The procedure is outlined below in Table 17. Note that all buffers disclosed in this example were made at room temperature and the pH of all buffers were measured at room temperature.

The steps of Example 5 are as follows: Equilibration: 0.2 g of the resin was inserted into a 15 ml Falcon tube and washed with 10 ml of 125 mM NaCl, 50 mM Tris HCl at pH 8.5±0.2. The suspension was centrifugated (HERAEUS MEGAFUGE 16R, THERMO SCIENTIFIC) for 10 min at 5500RPM and the supernatant was discarded. 9.6 g of LOAD was added to the washed/equilibrated resin and incubated for 15 h to 16 h at room temperature. The suspension was centrifugated for 10 min at 5500RPM. The supernatant was aliquoted and tested for AAV9 Antigen. The pellet was resuspended with 1 ml of cold 125 mM NaCl, 50 mM Tris HCl at pH 8.5±0.2 (+2° C. to +8° C.) and incubated for 30 min. The suspension was centrifugated for 10 min at 5500RPM. The supernatant was aliquoted and tested for AAV9 Antigen. The AAV9 contains vector DNA of approximately 2.6 to 3.0 kB. For elution, cold buffer was used, and the experiment was carried out in a cold room.

TABLE 17

| | | Capture Select | Capture Select |
|---|---|---|---|
| | | AAV9 | AAVX |
| Steps | Temperature | Cold elution | Cold elution |
| Resin | +18° C. to +30° C. | 0.2 g | 0.2 g |
| (Equilibrated) | | | |
| LOAD | +18° C. to +30° C. | 9.6 g | 9.6 g |
| Incubation | +18° C. to +30° C. | 15 to 16 h | |
| Centrifugation | +18° C. to +30° C. | 10 min/5500 RPM | |
| Supernatant | Supernatant was removed, Samples drawn, Pellet | | |
| | was processed further | | |
| Elution | +2° C. to +8° C. | 1 ml | 1 ml |
| | | [125 mM NaCl | [125 mM NaCl |
| | | 50 mM Tris HCl, | 50 mM Tris HCl, |
| | | pH 8.5 ± 0.2] | pH 8.5 ± 0.2] |

| | | |
|---|---|---|
| Incubation | +2° C. to +8° C. | 30 min |
| Centrifugation | +2° C. to +8° C. | 10 min/5500 RPM |

| | |
|---|---|
| Sample Eluate | Supernatant was removed, Samples drawn, Pellet |
| | was processed further |

The results from the above procedure, and particularly the percentage yield of AAV9 eluted from the column, are shown below in Table 18.

TABLE 18

| | | Capture Select | Capture Select |
|---|---|---|---|
| | | AAV9 | AAVX |
| | | [ % AAV9 | [ % AAV9 |
| Fraction | Temperature | Antigen] | Antigen] |
| LOAD | +18° C. to +30° C. | 100 | 100 |
| Supernatant | +18° C. to +30° C. | — | 6.2 |
| Sample Eluate | +2° C. to +8° C. | — | 36.7 |

Further, a second set used the different resins incubated at a lower temperature to investigate the binding properties at these conditions. The procedure is outline below in Table 19.

TABLE 19

| | | Capture Select | Capture Select |
|---|---|---|---|
| | | AAV9 | AAVX |
| Steps | Temperature | Cold elution | Cold elution |
| Resin (Equilibrated) | +2° C. to +8° C. | 0.2 g | 0.2 g |
| AAV9 containing | +2° C. to +8° C. | 9.6 g | 9.6 g |
| solution = LOAD | | | |

| | | |
|---|---|---|
| Incubation | +2° C. to +8° C. | 15 to 16 h |
| Centrifugation | +2° C. to +8° C. | 10 min/5500 RPM |

| | |
|---|---|
| Supernatant | Supernatant was removed, Samples drawn, |
| | Pellet was processed further |

The results from the above procedure, and particularly the percentage yield of AAV9 eluted from the column, are shown below in Table 20.

TABLE 20

| | | Capture Select | Capture Select |
|---|---|---|---|
| | | AAV9 | AAVX |
| | | [ % AAV9 | [ % AAV9 |
| Fraction | Temperature | Antigen] | Antigen] |
| LOAD | +2° C. to +8° C. | 100 | 100 |
| Supernatant | +2° C. to +8° C. | LOQ | 63.7 |

AAV9 binds on CaptureSelect™ AAV9 at every temperature range, elution cannot be triggered at the lower temperature. Thus, this example demonstrates that the elution at low temperature is a characteristic of the interaction between the AAV9 and AAVx affinity resin.

It was also observed that AAV8 and AAV6 did not elute from AAVx resin when the temperature was shifted from above +18° C. to below +8° C. Instead, AAV8 and AAV6 required harsher conditions (e.g., see Example 6 below) for them to elute from the AAVx affinity resin.

### Example 6

AAV8 production was developed in a HEK293 cell line after transfection with a triple plasmid system containing encoding cDNA of the protein of interest and VP1, VP2 and VP3 of AAV8. The clarified cell free culture supernatant was concentrated and diafiltrated with Pall Omega T-Series Cassette 100 kDa. The viral particles were loaded onto a membrane adsorber (MustangQ; Pall Part Number XT140MSTGQP05) at nonbinding conditions, i.e., in a solution comprising 125 mM NaCl and 50 mM TrisHCl at pH 8.5. A pH conditioned LOAD was obtained by adjusting the AAV8 containing flow through to a pH range between 8.3 to 8.7 with 25% HCl.

The following test procedure was undertaken. Note that all buffers disclosed in this example were made at room temperature and the pH of all buffers were measured at room temperature. First, a column containing POROS™ CaptureSelect™ AAVX Affinity Resin (Cat. No. 36742; Thermo Fisher) ID 16 mm, with a bed height of 52 mm, an area of 2.01 cm², and a volume of approximately 10.5 ml, was activated with 5 column volumes of a buffer comprising 100 mM glycine, 200 mM NaCl, at a pH of 2.0. The column was then equilibrated with at least five column volumes of 50 mM TrisHCl and 125 mM NaCl at pH 8.5. The pH conditioned LOAD was applied onto the column containing POROS™ CaptureSelect™ AAVX Affinity Resin.

The column was then washed with five column volumes of Wash 1 (W1): 50 mM TrisHCl and 125 mM NaCl, at pH 8.5 and at room temperature (18-26° C.). The column was then washed with five column volumes of Wash 2 (W2): 100 mM sodium acetate and 0.1% Tween 80, at pH 6.0 and at room temperature (18-26° C.). The column was next washed with five column volumes of Wash 1 (W1): 50 mM TrisHCl and 125 mM NaCl, at pH 8.5 and at room temperature (18-26° C.). The column was then washed with five column volumes of Wash 2 (W2): 100 mM sodium acetate and 0.1% Tween 80, at pH 6.0 and at room temperature (18-26° C.). For all of these wash steps, the linear flow rate was 60 cm/h.

For the elution, chromatography skid, column, and buffers were all lowered to below +8° C. via placing all items in a cooling cabinet (Unichromat 1500). Elution was first undertaken by applying five column volumes of W2 above, but at a lower temperature of between +2 to +8° C. at a linear flow rate of 5 cm/h. Five column volumes of the following secondary elution buffer were then applied to the column at the temperature of between +2 to +8° C.: 50 mM Glycine, 10 mM Histidine, 100 mM NaCl, 5% Trehalose, 0.005% Crillet 4 HP, pH 7.0 (ELT-buffer). Five column volumes of the following elution buffer were then applied to the column at the temperature of between +2 to +8° C.: 50 mM TrisHCl and 125 mM NaCl, at pH 8.5. The linear flow rate for these elution steps was 30 cm/h. Five column volumes of purified water were then applied to the column, again at a temperature of between +2 to +8° C. Gradient elution was then performed. 15 column volumes of a gradient from 1 mM to 20 mM HCl, 200 mM NaCl in purified water was applied at a linear flow rate of 20 cm/h and at a temperature of between +2 to +8° C.

The above procedure is described in more detail in Table 21, with “CV” indicating the number of column volumes of solution added in the step.

TABLE 21

| | | | | Amount | | |
|---|---|---|---|---|---|---|
| Step | | Buffer | Buffer comp. | (CV) | Flow rate | Temp |
| 1 | Activation | REG2 | 100 mM Glycine, | 5 | 60 cm/h | +18-26° C. |
| | | | 200 mM NaCl | | | |
| | | | pH 2.0 | | | |
| 2 | Equilibration | W1 | 125 mM NaCl/50 mM | 10 | 60 cm/h | +18-26° C. |
| | | | TrisHCl | | | |
| | | | pH 8.5 | | | |
| 3 | Product load | Conditioned | 125 mM NaCl/50 mM | — | 60 cm/h | +18-26° C. |
| | | AAV8 | TrisHCl | | | |
| | | containing | pH 8.5 | | | |
| | | solution | | | | |
| 4 | Wash 1 | W1 | 125 mM NaCl/50 mM | 5 | 60 cm/h | +18-26° C. |
| | | | TrisHCl | | | |
| | | | pH 8.5 | | | |
| 5 | Wash 2 | W2 | 100 mM NaAcetat/ | 5 | 60 cm/h | +18-26° C. |
| | | | 0.1% Tween 80 | | | |
| | | | pH 6.0 | | | |
| 6 | Wash 3 | W1 | 125 mM NaCl/50 mM | 5 | 60 cm/h | +18-26° C. |
| | | | TrisHCl | | | |
| | | | pH 8.5 | | | |
| 7 | Wash 4 | W2 | 100 mM NaAcetat/ | 5 | 60 cm/h | +18-26° C. |
| | | | 0.1% Tween 80 | | | |
| | | | pH 6.0 | | | |
| 8 | Elution 1 | W2 | 100 mM NaAcetat/ | 10 | 5 cm/h | +2-8° C. |
| | Cool down | | 0.1% Tween 80 | | | |
| | | | pH 6.0 | | | |
| 9 | Elution 2 | ELT | 50 mM Glycin,10 mM | 5 | 30 cm/h | +2-8° C. |
| | | | Histidine, 100 mM | | | |
| | | | NaCl, 5% Trehalose, | | | |
| | | | 0.005% Crillet 4 HP, pH | | | |
| | | | 7.0 (ELT-buffer) | | | |
| 10 | Elution 3 | W1 | 125 mM NaCl/50 mM | 5 | 30 cm/h | +2-8° C. |
| | | | TrisHCl | | | |
| | | | pH 8.5 | | | |
| 11 | Wash 5 | PW | Purified water | 5 | 30 cm/h | +2-8° C. |
| | | | Gradient from 1 mM to | | | |
| 12 | Elution 4 | Gradient | 20 mM HCl/200 mM | 15 | 20 cm/h | +2-8° C. |
| | | | NaCl in | | | |
| | | | Purified water | | | |
| 13 | Strip 1 | STR1 | 20 mM HCl | 5 | 60 cm/h | +2-8° C. |
| | | | 200 mM NaCl in | | | |
| | | | Purified water | | | |
| 14 | Wash 6 | STR2 | 125 mM NaCl/50 mM | 5 | 60 cm/h | +2-8° C. |
| | | | TrisHCl | | | |
| | | | pH 8.5 | | | |

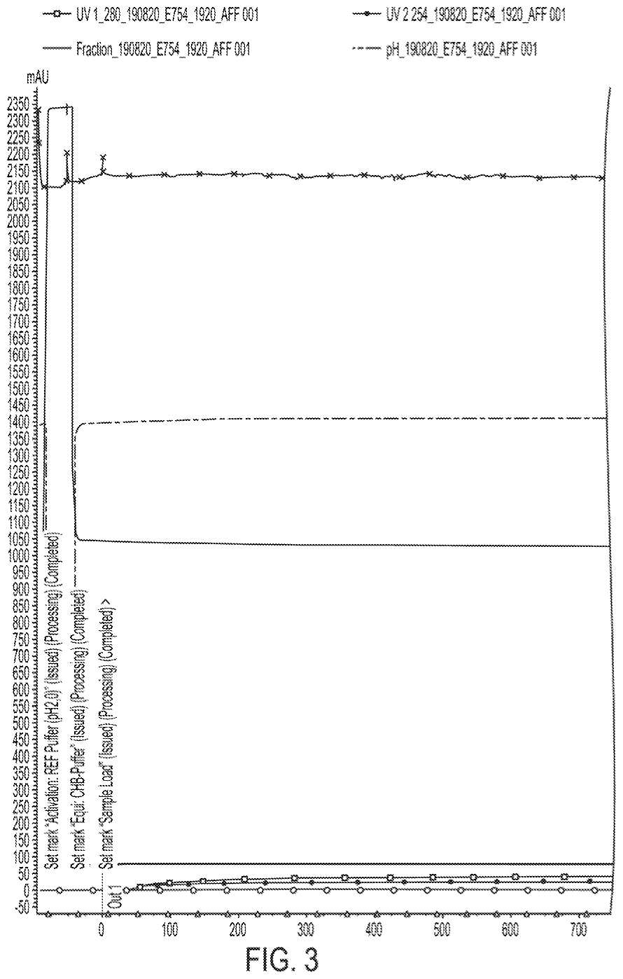

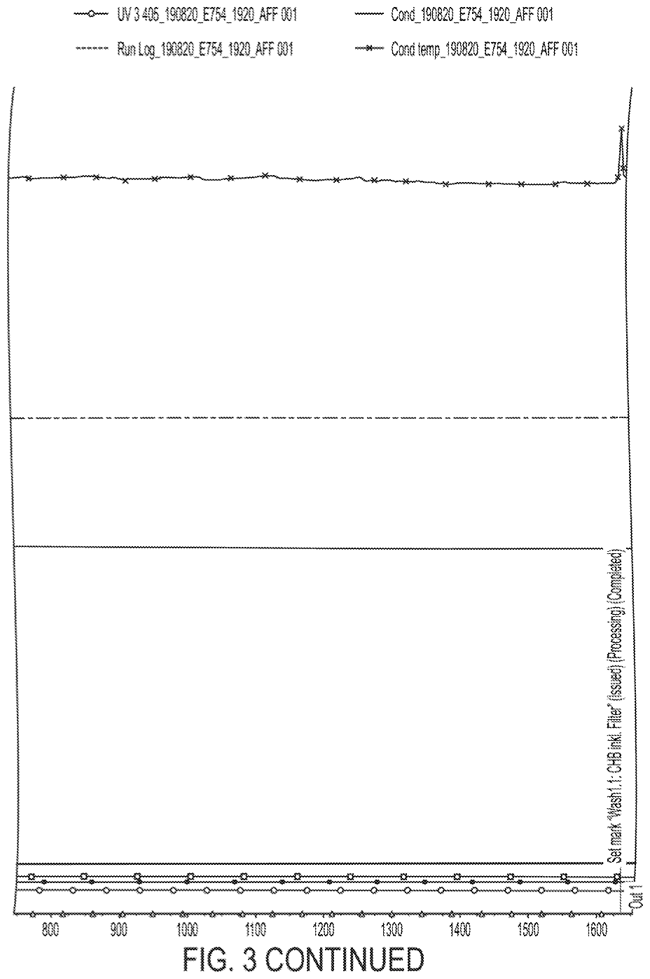

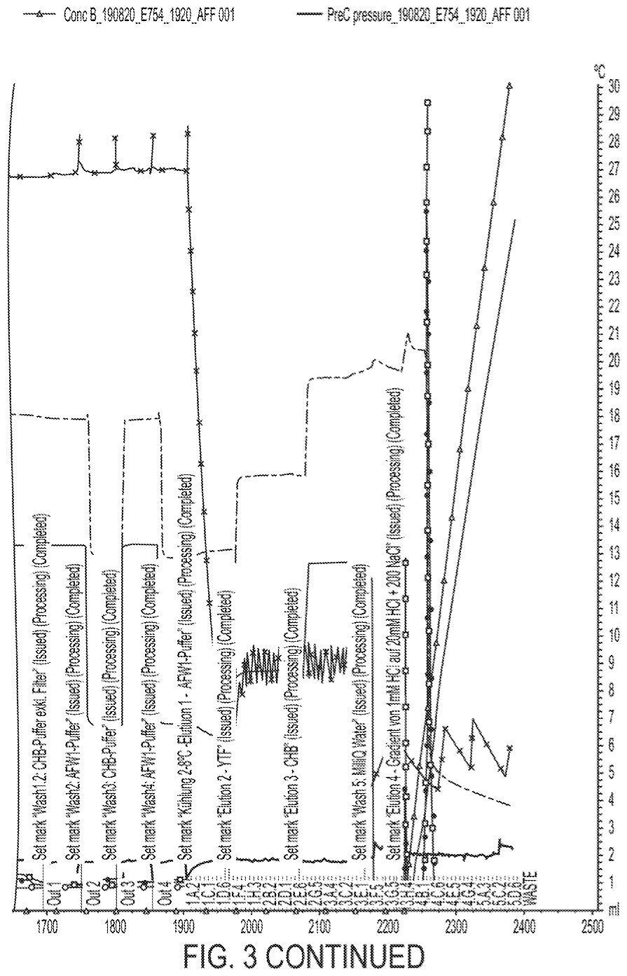

The results from the above procedure, and particularly the percentage yield of AAV8 eluted from the column, are shown below in Table 22 and FIG. 3.

TABLE 22

| | | ITR- | Total | | AAV8 | AAV8 | | Ratio |
|---|---|---|---|---|---|---|---|---|
| | | qPCR | ITR- | ITR- | Antigen | Total | | qPCR/ |
| | Amount | vg/ml × | qPCR | qPCR | cp/ml × | Antigen | Antigen | Antigen |
| | [g] | 10¹¹ | vg × 10¹¹ | [%] | 10¹¹ | cp × 10¹¹ | [%] | vg/cp |
| LOAD | 1636.44 | 19.00 | 31092.36 | 100.0% | 23.2 | 37965.408 | 100.00% | 0.819 |
| FT/W1 | 1756.25 | — | — | — | <0.0159 | <27.924 | — | — |
| W2 | 54.87 | — | — | — | <0.0159 | <0.872 | — | — |
| W3 | 55.22 | — | — | — | <0.0159 | <0.878 | — | — |
| W4 | 54.93 | — | — | — | <0.0159 | <0.873 | — | — |
| E1 | 24.87 | — | — | — | <0.0159 | <0.395 | — | — |
| E2 | 23.78 | — | — | — | <0.0159 | <0.378 | — | — |
| E3 | 15.77 | — | — | — | <0.0159 | <0.251 | — | — |
| E4 | 41.02 | pool | 17182 | 55.26 | pool | 28774 | 75.79 | 0.597 |

As shown, AAV8 cannot be eluted from AAVx with the temperature shift to +2-+8° C.

### Example 7

The following procedure is an example of a single wash protocol. First, a column containing POROS™ CaptureSelect™ AAVX Affinity Resin (Cat. No. 36742; Thermo Fisher) ID 16 mm, with a bed height of 50±0.5 mm, an area of 2.01 cm², and a volume of approximately 10 ml, is activated with five column volumes of a buffer comprising 100 mM glycine, 200 mM NaCl, at a pH of 2.0. The column is then equilibrated with 10 column volumes of 50 mM TrisHCl and 125 mM NaCl at pH 8.5. The pH conditioned LOAD is applied onto the column containing POROS™ CaptureSelect™ AAVX Affinity Resin.

The column is then washed with five column volumes of Wash 1 (W1): 50 mM TrisHCl and 125 mM NaCl, at pH 8.5 and at room temperature (18-26° C.). Elution is undertaken by applying five to 10 column volumes of W1 above, but at a lower temperature of between +2 to +8° C. The column is then stripped.

The above procedure is described in more detail in Table 23, with “CV” indicating the number of column volumes of solution added in the step.

| | | | | Amount | | |
|---|---|---|---|---|---|---|
| Step | | Buffer | Buffer comp. | (CV) | Flow rate | Temp |
| 1 | Activation | REG2 | 100 mM Glycine, | 5 | 60 cm/h | +18-26° C. |
| | | | 200 mM NaCl | | | |
| | | | pH 2.0 | | | |
| 2 | Equilibration | W1 | 125 mM NaCl/50 mM | 10 | 60 cm/h | +18-26° C. |
| | | | TrisHCl | | | |
| | | | pH 8.5 | | | |
| 3 | Product | Conditioned | 125 mM NaCl/50 mM | — | 60 cm/h | +18-26° C. |
| | load | AAV8 | TrisHCl | | | |
| | | containing | pH 8.5 | | | |
| | | solution | | | | |
| 4 | Wash 1 | W1 | 125 mM NaCl/50 mM | 5 | 60 cm/h | +18-26° C. |
| | | | TrisHCl | | | |
| | | | pH 8.5 | | | |
| 5 | Elution 1 | W1 | 125 mM NaCl/50 mM | 5 to 10 | 5 cm/h | +2-8° C. |
| | Cool down | | TrisHCl | | | |
| | | | pH 8.5 | | | |
| 6 | STRIP | REG2 | 100 mM Glycine | 5 | 60 cm/h | +2-8° C. |
| | | | 200 mM NaCl | | | |
| | | | pH 2.0 | | | |

### Example 8

The below sequence illustrates an example of an AAV9 VP1 sequence according to some embodiments of the present disclosure (SEQ ID NO: 1).

```
                10 20 30 40 50
MAADGYLPDW LEDNLSEGIR EWWALKPGAP QPKANQQHQD NARGLVLPGY
                60 70 80 90 100
KYLGPGNGLD KGEPVNAADA AALEHDKAYD QQLKAGDNPY LKYNHADAEF
              110 120 130 140 150
QERLKEDTSF GGNLGRAVFQ AKKRLLEPLG LVEEAAKTAP GKKRPVEQSP
              160 170 180 190 200
QEPDSSAGIG KSGAQPAKKR LNFGQTGDTE SVPDPQPIGE PPAAPSGVGS
              210 220 230 240 250
LTMASGGGAP VADNNEGADG VGSSSGNWHC DSQWLGDRVI TTSTRTWALP
              260 270 280 290 300
TYNNHLYKQI SNSTSGGSSN DNAYFGYSTP WGYFDFNRFH CHFSPRDWQR
              310 320 330 340 350
LINNNWGFRP KRLNFKLFNI QVKEVTDNNG VKTIANNLTS TVQVFTDSDY
              360 370 380 390 400
QLPYVLGSAH EGCLPPFPAD VFMIPQYGYL TLNDGSQAVG RSSFYCLEYF
              410 420 430 440 450
PSQMLRTGNN FQFSYEFENV PFHSSYAHSQ SLDRLMNPLI DQYLYYLSKT
              460 470 480 490 500
INGSGQNQQT LKFSVAGPSN MAVQGRNYIP GPSYRQQRVS TTVTQNNNSE
              510 520 530 540 550
FAWPGASSWA LNGRNSLMNP GPAMASHKEG EDRFFPLSGS LIFGKQGTGR
              560 570 580 590 600
DNVDADKVMI TNEEEIKTTN PVATESYGQV ATNHQSAQAQ AQTGWVQNQG
              610 620 630 640 650
ILPGMVWQDR DVYLQGPIWA KIPHTDGNFH PSPLMGGFGM KHPPPQILIK
              660 670 680 690 700
NTPVPADPPT AFNKDKLNSF ITQYSTGQVS VEIEWELQKE NSKRWMPEIQ
              710 720 730
YTSNYYKSNN VEFAVNTEGV YSEPRPIGTR YLTRNL
```

The below sequence illustrates an example of an AAV9 VP2 sequence according to some embodiments of the present disclosure, wherein the AAV9 VP2 sequence comprises the sequence of SEQ ID NO: 2.

```
138 140 150
              TAP GKKRPVEQSP
              160 170 180 190 200
QEPDSSAGIG KSGAQPAKKR LNFGQTGDTE SVPDPQPIGE PPAAPSGVGS
              210 220 230 40 250
LTMASGGGAP VADNNEGADG VGSSSGNWHC DSQWLGDRVI TTSTRTWALP
              260 270 280 290 300
TYNNHLYKQI SNSTSGGSSN DNAYFGYSTP WGYFDFNRFH CHFSPRDWQR
              310 320 330 340 350
LINNNWGFRP KRLNFKLFNI QVKEVTDNNG VKTIANNLTS TVQVFTDSDY
              360 370 380 390 400
QLPYVLGSAH EGCLPPFPAD VFMIPQYGYL TLNDGSQAVG RSSFYCLEYF
              410 420 430 440 450
PSQMLRTGNN FQFSYEFENV PFHSSYAHSQ SLDRLMNPLI DQYLYYLSKT
              460 470 480 490 500
INGSGQNQQT LKFSVAGPSN MAVQGRNYIP GPSYRQQRVS TTVTQNNNSE
              510 520 530 540 550
FAWPGASSWA LNGRNSLMNP GPAMASHKEG EDRFFPLSGS LIFGKQGTGR
              560 570 580 590 600
DNVDADKVMI TNEEEIKTTN PVATESYGQV ATNHQSAQAQ AQTGWVQNQG
              610 620 630 640 650
ILPGMVWQDR DVYLQGPIWA KIPHTDGNFH PSPLMGGFGM KHPPPQILIK
              660 670 680 690 700
NTPVPADPPT AFNKDKLNSF ITQYSTGQVS VEIEWELQKE NSKRWNPEIQ
              710 720 730
YTSNYYKSNN VEFAVNTEGV YSEPRPIGTR YLTRNL
```

The below sequence illustrates an example of an AAV9 VP2 sequence according to some embodiments of the present disclosure, wherein the AAV9 VP3 sequence comprises the sequence of SEQ ID NO: 3.

```
203 210 220 230 240 250
    MASGGGAP VADNNEGADG VGSSSGNWHC DSQWLGDRVI TTSTRTWALP
              260 270 280 290 300
TYNNHLYKQI SNSTSGGSSN DNAYFGYSTP WGYFDFNRFH CHFSPRDWQR
              310 320 330 340 350
LINNNWGFRP KRLNFKLFNI QVKEVTDNNG VKTIANNLTS TVQVFTDSDY
              360 370 380 390 400
QLPYVLGSAH EGCLPPFPAD VFMIPQYGYL TLNDGSQAVG RSSFYCLEYF
              410 420 430 440 450
PSQMLRTGNN FQFSYEFENV PFHSSYAHSQ SLDRLMNPLI DQYLYYLSKT
              460 470 480 490 500
INGSGQNQQT LKFSVAGPSN MAVQGRNYIP GPSYRQQRVS TTVTQNNNSE
              510 520 530 540 550
FAWPGASSWA LNGRNSLMNP GPAMASHKEG EDRFFPLSGS LIFGKQGTGR
              560 570 580 590 600
DNVDADKVMI TNEEEIKTTN PVATESYGQV ATNHQSAQAQ AQTGWVQNQG
              610 620 630 640 650
ILPGMVWQDR DVYLQGPIWA KIPHTDGNFH PSPLMGGFGM KHPPPQILIK
              660 670 680 690 700
NTPVPADPPT AFNKDKLNSF ITQYSTGQVS VEIEWELQKE NSKRWNPEIQ
              710 720 730
YTSNYYKSNN VEFAVNTEGV YSEPRPIGTR YLTRNL
```

All references, including publications, patent applications, and patents, cited herein are hereby incorporated by reference to the same extent as if each reference were individually and specifically indicated to be incorporated by reference and were set forth in its entirety herein.

What is claimed is:

1. A method of purifying an adeno-associated virus (AAV) comprising
(a) loading an AAV containing solution onto an affinity resin targeted against the AAV at room temperature and under conditions that allow binding between the AAV in the solution and the affinity resin;
(b) undertaking at least one wash step at room temperature; and
(c) eluting the AAV from the affinity resin at a temperature of less than 18° C.

2. The method of claim 1, wherein the temperature in step (c) is between 1° C. and 12° C.

3. The method of claim 1, wherein the temperature in step (c) is between 2° C. and 8° C.

4. The method of any one of claims 1 to 3, further comprising contacting the AAV containing solution with an anion exchanger and eluting the AAV containing solution from the anion exchanger prior to loading the AAV containing solution onto the affinity resin.

5. The method of any one of claims 1 to 4, wherein at least two wash steps are performed at room temperature.

6. The method of any one of claims 1 to 5, wherein at least three wash steps are performed at room temperature.

7. The method of any one of claims 1 to 6, wherein at least four wash steps are performed at room temperature.

8. The method of claim 5, wherein two wash steps are performed.

9. The method of claim 6, wherein three wash steps are performed.

10. The method of claim 7, wherein four wash steps are performed.

11. The method of any one of claims 5 to 10, wherein the wash steps are performed in succession.

12. The method of any one of claims 1 to 11, wherein at least one wash buffer comprises from about 10 mM to about 200 mM TrisHCl and from about 50 mM to about 500 mM salt.

13. The method of claim 12, wherein at least one wash buffer comprises from about 25 mM to about 100 mM TrisHCl and from about 75 mM to about 250 mM salt

14. The method of claim 13, wherein at least one wash buffer comprises from about 40 mM to about 60 mM TrisHCl and from about 100 mM to about 150 mM salt.

15. The method of any one of claims 12 to 14, wherein at least one wash buffer has a pH from about 7.5 to about 9.2, about 8.0 to about 9.0, or about 8.0 to about 8.8.

16. The method of claim 15, wherein at least one wash buffer comprises about 50 mM TrisHCl and about 125 mM salt, and has a pH of about 8.5.

17. The method of any one of claims 1 to 16, wherein at least one wash buffer comprises from about 10 mM to about 2000 mM sodium acetate and from about 0.001% to about 1% (w/w) polysorbate 80.

18. The method of claim 17, wherein at least one wash buffer comprises from about 50 mM to about 200 mM sodium acetate and from about 0.005% to about 0.3% (w/w) polysorbate 80.

19. The method of claim 18, wherein at least one wash buffer comprises from about 90 mM to about 110 mM sodium acetate and from about 0.05% to about 0.2% (w/w) polysorbate 80.

20. The method of any one of claims 17 to 19, wherein at least one wash buffer has a pH from about 5.0 to about 7.4, about 5.5 to about 7.0, or about 5.5 to about 6.5.

21. The method of claim 20, wherein at least one wash buffer comprises about 100 mM sodium acetate and about 0.1% (w/w) polysorbate 80, and has a pH of about 6.0.

22. The method of any one of claims 1 to 21, wherein at least one wash buffer comprises from about 10 mM to about 200 mM TrisHCl and from about 10% to about 75% (w/w) ethylene glycol.

23. The method of claim 22, wherein at least one wash buffer comprises from about 25 mM to about 100 mM TrisHCl and from about 25% to about 70% (w/w) ethylene glycol.

24. The method of claim 23, wherein at least one wash buffer comprises from about 40 mM to about 60 mM TrisHCl and from about 40% to about 60% (w/w) ethylene glycol.

25. The method of any one of claims 22 to 24, wherein at least one wash buffer has a pH from about 7.5 to about 9.2, about 8.0 to about 9.0, or about 8.0 to about 8.8.

26. The method of claim 25, wherein at least one wash buffer comprises about 50 mM TrisHCl and about 50% (w/w) ethylene glycol, and has a pH of about 8.5.

27. The method of any one of claims 1 to 26, wherein at least one wash buffer comprises from about 10 mM to about 200 mM glycine, about 1 mM to about 100 mM histidine, about 20 mM to about 500 mM salt, about 1% to about 10% (w/w) trehalose and about 0.0005% to about 1% (w/w) polysorbate 80.

28. The method of claim 27, wherein at least one wash buffer comprises from about 30 mM to about 80 mM glycine, about 5 mM to about 20 mM histidine, about 50 mM to about 200 mM salt, about 3% to about 8% (w/w) trehalose and about 0.001% to about 0.1% (w/w) polysorbate 80.

29. The method of claim 28, wherein at least one wash buffer comprises from about 40 mM to about 60 mM glycine, about 5 mM to about 15 mM histidine, about 90 mM to about 110 mM salt, about 4% to about 6% (w/w) trehalose and about 0.001% to about 0.05% (w/w) polysorbate 80.

30. The method of any one of claims 27 to 29, wherein at least one wash buffer has a pH from about 6.0 to about 8.0, about 6.5 to about 7.5, or about 7.0 to about 7.4.

31. The method of claim 30, wherein at least one wash buffer comprises about 50 mM glycine, about 10 mM histidine, about 100 mM salt, about 5% (w/w) trehalose, about 0.005% (w/w) polysorbate 80, and has a pH of about 7.0.

32. The method of any one of claims 1 to 31, wherein at least one wash buffer comprises from about 1 mM to about 200 mM TrisHCl, from about 50 mM to about 500 mM salt, and from about 0.001% to about 1% (w/w) polysorbate 80.

33. The method of claim 32, wherein at least one wash buffer comprises from about 5 mM to about 50 mM TrisHCl, from about 75 mM to about 250 mM salt, and from about 0.005% to about 0.3% (w/w) polysorbate 80.

34. The method of claim 33, wherein at least one wash buffer comprises from about 10 mM to about 30 mM TrisHCl, from about 140 mM to about 160 mM salt, and from about 0.05% to about 0.2% (w/w) polysorbate 80.

35. The method of any one of claims 32 to 34, wherein at least one wash buffer has a pH from about 6.0 to about 8.8, about 6.5 to about 8.5, or about 7.0 to about 8.0.

36. The method of claim 35, wherein at least one wash buffer comprises about 20 mM TrisHCl, about 150 mM salt, and about 0.1% (w/w) polysorbate 80, and has a pH of about 7.4.

37. The method of any one of claims 1 to 36, wherein step (c) comprises eluting the AAV with at least one elution buffer.

38. The method of claim 37, wherein at least one elution buffer is the same as at least one of the wash buffers.

39. The method of claim 38, wherein at least one elution buffer is the same as the last wash buffer used in the final wash step before eluting the AAV in step (c).

40. The method of claim 38, wherein the first elution buffer is the same as the last wash buffer used in the final wash step before eluting the AAV in step (c).

41. The method of any one of claims 37 to 40, wherein at least one elution buffer comprises from about 10 mM to about 200 mM glycine, about 1 mM to about 100 mM histidine, about 20 mM to about 500 mM salt, about 1% to about 10% (w/w) trehalose, and about 0.0005% to about 1% (w/w) polysorbate 80.

42. The method of claim 41, wherein at least one elution buffer comprises from about 30 mM to about 80 mM glycine, about 5 mM to about 20 mM histidine, about 50 mM to about 200 mM salt, about 3% to about 8% trehalose, and about 0.001% to about 0.1% (w/w) polysorbate 80.

43. The method of claim 42, wherein at least one elution buffer comprises from about 40 mM to about 60 mM glycine, about 5 mM to about 15 mM histidine, about 90 mM to about 110 mM salt, about 4% to about 6% (w/w) trehalose, and about 0.001% to about 0.05% (w/w) polysorbate 80.

44. The method of any one of claims 41 to 43, wherein at least one elution buffer has a pH from about 6.0 to about 8.0, about 6.5 to about 7.5, or about 7.0 to about 7.4.

45. The method of claim 44, wherein at least one elution buffer comprises about 50 mM glycine, about 10 mM histidine, about 100 mM salt, about 5% (w/w) trehalose, and about 0.005% (w/w) polysorbate 80, and has a pH of about 7.0.

46. The method of any one of claims 37 to 45, wherein at least one elution buffer comprises from about 1 mM to about 200 mM TrisHCl, from about 50 mM to about 500 mM salt, and from about 0.001% to about 1% (w/w) polysorbate 80.

47. The method of claim 46, wherein at least one elution buffer comprises from about 5 mM to about 50 mM TrisHCl, from about 75 mM to about 250 mM salt, and from about 0.005% to about 0.3% (w/w) polysorbate 80.

48. The method of claim 47, wherein at least one elution buffer comprises from about 10 mM to about 30 mM TrisHCl, from about 140 mM to about 160 mM salt, and from about 0.05% to about 0.2% (w/w) polysorbate 80.

49. The method of any one of claims 46 to 48, wherein at least one elution buffer has a pH from about 6.0 to about 8.8, about 6.5 to about 8.5, or about 7.0 to about 8.0.

50. The method of claim 49, wherein at least one elution buffer comprises about 20 mM TrisHCl, about 150 mM salt, and about 0.1% (w/w) polysorbate 80 and has a pH of about 7.4.

51. The method of any one of claims 1 to 50, wherein a first and third wash steps comprise applying to the affinity resin a buffer comprising from about 10 mM to about 200 mM TrisHCl and from about 50 mM to about 500 mM salt with a pH from about 7.5 to about 9.2, and
wherein a second wash step comprises applying to the affinity resin a buffer comprising from about 10 mM to about 2000 mM sodium acetate and from about 0.001% to about 1% (w/w) polysorbate 80 with a pH from about 5.0 to about 7.4.

52. The method of any one of claims 1 to 50, wherein a first and third wash steps comprise applying to the affinity resin a buffer compressing from about 10 mM to about 2000 mM sodium acetate and from about 0.00%1 to about 1% (w/w) polysorbate 80 with a pH from about 5.0 to about 7.4, and
wherein a second wash step comprises applying to the affinity resin a buffer comprising from about 10 mM to about 200 mM TrisHCl and from about 50 mM to about 500 mM salt with a pH from about 7.5 to about 9.2.

53. The method of any one of claims 1 to 50, wherein a first and third wash steps comprise applying to the affinity resin a buffer compressing from about 10 mM to about 200 mM TrisHCl and from about 50 mM to about 500 mM salt with a pH from about 7.5 to about 9.2, and
wherein a second and fourth wash step comprises applying to the affinity resin a buffer comprising about 10 mM to about 2000 mM sodium acetate and from about 0.001% to about 1% (w/w) polysorbate 80 with a pH from about 5.0 to about 7.4.

54. The method of any one of claims 1 to 50, wherein a first and third wash steps comprise applying to the affinity resin a buffer comprising from about 10 mM to about 2000 mM sodium acetate and from about 0.001% to about 1% (w/w) polysorbate 80 with a pH from about 5.0 to about 7.4, and
wherein a second and fourth wash step comprises applying to the affinity resin a buffer compressing from about 10 mM to about 200 mM TrisHCl and from about 50 mM to about 500 mM salt with a pH from about 7.5 to about 9.2.

55. The method of claim 51 or claim 53, wherein a first and third buffer used in the first and third wash steps, respectively, each comprise about 50 mM TrisHCl and about 125 mM salt, and has a pH of about 8.5.

56. The method of claim 52 or claim 54, wherein a first and third buffer used in the first and third wash steps, respectively, each comprise about 100 mM sodium acetate, about 0.1% (w/w) polysorbate 80, and has a pH of about 6.0.

57. The method of claim 51 or claim 53, wherein a second buffer used in the second was step comprises about 100 mM sodium acetate, about 0.1% (w/w) polysorbate 80, and has a pH of about 6.0.

58. The method of claim 52 or claim 54, wherein the second buffer used in the second was step comprises about 50 mM TrisHCl and about 125 mM salt, and has a pH of about 8.5.

59. The method of claim 53, wherein the fourth buffer used in the fourth was step comprises about 100 mM sodium acetate, about 0.1% (w/w) polysorbate 80, and has a pH of about 6.0.

60. The method of claim 54, wherein the fourth buffer used in the fourth was step comprises about 50 mM TrisHCl and about 125 mM salt, and has a pH of about 8.5.

61. The method of claim 51 or claim 54, wherein step (c) comprises applying to the affinity resin a buffer comprising about 10 mM to about 200 mM TrisHCl and from about 50 mM to about 500 mM salt, and has a pH from about 7.5 to about 9.2.

62. The method of claim 52 or claim 53, wherein step (c) comprises applying to the affinity resin a buffer comprising about 10 mM to about 2000 mM sodium acetate and from about 0.001% to about 1% (w/w) polysorbate 80, and has a pH from about 5.0 to about 7.4.

63. The method of claim 61, wherein the buffer comprises about 50 mM TrisHCl and about 125 mM salt, and has a pH of about 8.5.

64. The method of claim 62, wherein the buffer comprises about 100 mM sodium acetate, about 0.1% (w/w) polysorbate 80, and has a pH of about 6.0.

65. The method of any one of claims 12 to 64, wherein the salt is selected from NaCl, KCl, MgCl₂, CaCl₂), Sodium Citrate, LiCl, CsCl, Sodium Acetate, and a combination of one or more of NaCl, KCl, MgCl₂, CaCl₂), Sodium Citrate, LiCl, CsCl, and Sodium Acetate.

66. The method of claim 65, wherein the salt is NaCl.

67. The method of any one of claims 12 to 16, 51 to 55, 58, 60, or 61, wherein the buffer comprises about 50 mM TrisHCl and about 125 mM NaCl with a pH of about 8.5.

68. The method of any one of claims 1 to 67, wherein the AAV obtained from the eluting step has a purity level of 99.0% or greater.

69. The method of any one of claims 1 to 68, wherein the AAV obtained from the eluting step has a purity level of 99.9% or greater.

70. The method of any one of claims 1 to 69, wherein at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% of the AAV capsids eluted from the elution step (c) are full AAV capsids.

71. The method of any one of claims 1 to 70, wherein the affinity resin is AAVx resin.

72. The method of any one of claims 1 to 71, wherein the AAV is AAV9.

73. The method of claim 72, wherein the AAV9 comprises a peptide comprising the sequence of SEQ ID NO: 1, SEQ ID NO: 2, and/or SEQ ID NO: 3.

74. An AAV product produced by a method according to any one of claims 1 to 73.
